(19) United States
(12) Patent Application Publication
(10) Pub. No.: US 2012/0220564 A1
(43) Pub. Date: Aug. 30, 2012

(54) SELECTIVE CALCIUM CHANNEL MODULATORS

(75) Inventors: Hassan Pajouhesh, West Vancouver (CA); Yongbao Zhu, Langley (CA); Yuanxi Zhou, Richmond (CA); Michael E. Grimwood, North Vancouver (CA); Eric Simonson, Vancouver (CA)

(73) Assignee: Zalicus Pharmaceuticals Ltd., Vancouver, BC (CA)

(21) Appl. No.: 13/496,648

(22) PCT Filed: Sep. 17, 2010
(86) PCT No.: PCT/CA2010/001471

§ 371 Date: May 2, 2012

Related U.S. Application Data
(60) Provisional application No. 61/243,892, filed on Sep. 18, 2009.

(51) Int. Cl. A61K 31/4523; A61K 31/4545; C07D 211/34; A61K 31/445; C07D 413/06; A61K 31/5377; C07D 401/12; C07D 413/12; A61K 31/454; C07D 207/09; A61K 31/40; C07D 205/04; A61K 31/397; C07D 211/48; A61P 29/00; A61P 25/16; A61P 25/24; A61P 25/18; A61P 27/16; A61P 25/06; C07D 401/06

(52) U.S. Cl. 514/210.18; 546/189; 514/316; 546/245; 514/330; 544/130; 514/235.5; 546/194; 514/318; 546/209; 514/326; 546/208; 548/539; 514/423; 548/953; 514/210.17; 546/188; 546/242; 514/327

(57) ABSTRACT

Methods and compounds effective in ameliorating conditions characterized by unwanted calcium channel activity, particularly unwanted T-type calcium channel activity are disclosed using a series of compounds containing N-acylated cyclic amines linked to an aπl ring as shown in formula (I).

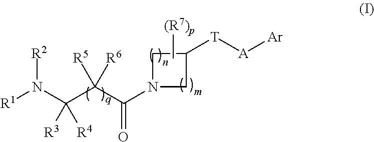

### CROSS-REFERENCE TO RELATED APPLICATIONS

This application claims benefit of U.S. Provisional Application No. 61/243,892, filed Sep. 18, 2009, which is hereby incorporated by reference.

### TECHNICAL FIELD

The invention relates to compounds useful in treating conditions associated with calcium channel function, and particularly conditions associated with T-type calcium channel activity. More specifically, the invention concerns compounds containing substituted amino N-piperidinyl acetamide derivatives that are useful in the prophylactic care, amelioration, or diagnosis of conditions associated with ion channel function, such as the treatment or prevention of conditions such as cardiovascular disease, obesity, epilepsy and pain.

### BACKGROUND OF THE INVENTION

Calcium channels mediate a variety of normal physiological functions and are also implicated in a number of human disorders. Examples of calcium-mediated human disorders include but are not limited to congenital migraine, cerebellar ataxia, angina, epilepsy, hypertension, ischemia, and some arrhythmias (see, e.g., Janis et al., Ion Calcium Channels: Their Properties, Functions, Regulation and Clinical Relevance (1991) CRC Press, London). T-type, or low voltage-activated, channels describe a broad class of molecules that transiently activate at negative potentials and are highly sensitive to changes in resting potential and are involved in various medical conditions. For example, in mice lacking the gene expressing the 3.1 subunit (Ca_{V} 3.1), resistance to absence seizures was observed (Kim et al., Mol Cell Neurosci 18(2): 235-245, 2001). Other studies have also implicated the 3.2 subunit (Ca_{V} 3.2) in the development of epilepsy (Su et al., J Neurosci 22: 3645-3655, 2002).

Novel allosteric modulators of calcium channels, e.g., T-type calcium channels, are thus desired. Modulators may affect the kinetics and/or the voltage potentials of, e.g., the Ca_{V}3.2 channel.

The invention provides compounds that act at these T-type calcium channels and are useful to treat various conditions associated with these calcium channels, such as pain and epilepsy. It also provides pharmaceutical compositions containing these compounds and methods to use them either alone or in combination with other pharmaceutical agents.

### SUMMARY OF THE INVENTION

In a first aspect, the invention features a compound according to the following formula,

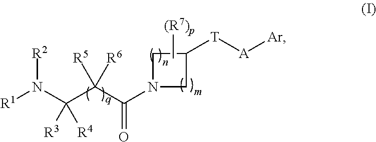

or a pharmaceutically acceptable salt, solvate, or prodrug thereof, or a stereoisomer thereof, or a conjugate thereof, where

Ar is phenyl or a 5-6 membered heteroaryl ring containing at least one heteroatom selected from N, O and S as a ring member, and optionally substituted with at least one R⁸, R⁹, or R¹⁰;

T is CH₂, O, or NR¹;

A is [T]-C(O)—NR¹ or [T]-NR¹—C(O)— or [T]-C(O)—O—, or [T]-O—C(O)—, —NR¹—C(O)—NR¹—; [T]-O—C(O)—NR¹—; or [T]-NR¹—C(O)—O—; where [T] indicates which atom of A is linked to T in Formula (I);

R¹ and R² are independently selected from the group consisting of H, optionally substituted C1-C6 alkyl, C1-C6 optionally substituted alkylsulfonyl, and optionally substituted C1-C6 acyl;

R³, R⁴, R⁵, R⁶ and R⁷ are independently selected from H, optionally substituted C1-C6 alkyl, halo, hydroxy, CN, optionally substituted C1-C6 alkoxy, and optionally substituted C1-C6 heteroalkyl;

where R² and R³, or R³ and R⁴, or R⁴ and R⁵, or R⁶ and R², or two R⁶ if two R⁶ are present (any one of these pairs, but not more than one pair) can optionally be taken together to form a non-aromatic 5-6 membered ring, which can optionally include up to two heteroatoms selected from N, O and S as ring members;

and two R⁷ can optionally be taken together to form a non-aromatic 5-6 membered ring, which can optionally include a heteroatom selected from N, O and S as a ring member;

m and n are independently 1 or 2;

p is 0-2;

q is 0, 1 or 2;

R⁸, R⁹, and R¹⁰ are optional substituents that are independently selected from the group consisting of H, halogen, CN, —SO₂—(C1-C4 alkyl), optionally substituted C1-C6 alkyl, optionally substituted C1-C6 alkoxy, optionally substituted aryl, and optionally substituted heteroaryl, provided at least one of R⁸, R⁹, and R¹⁰ is present and is not H.

In some embodiments, the compound ha a structure according to the following formula,

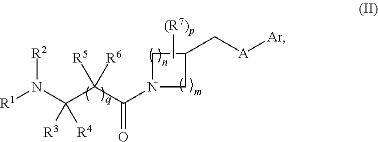

where

q is 0 or 1;

A is an amide of the formula —NR¹—C(O)— or —C(O)—NR¹—; or a urea of the formula —NR¹—C(O)—NR¹—; or a carbamate of the formula —O—C(O)—NR¹— or —NR¹—C(O)—O—;

Ar is a phenyl or pyridyl group that is substituted by R⁸, R⁹, and R¹⁰, or Ar is a 1,3,4-oxadiazolyl group optionally substituted by R⁸; and

R⁸, R⁹, and R¹⁰ are, independently, selected from the group consisting of H, F, Cl, Br, CN, optionally substituted C1-C6 alkyl, optionally substituted C1-C6 alkoxy, and —SO₂-(optionally substituted C1-C4 alkyl).

In further embodiments, the compound has a structure according to the following formula,

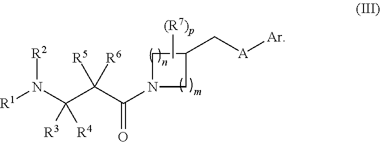

In other embodiments, R⁸, R⁹, and R¹⁰ are, independently, selected from F, Cl, Br, Me, OMe, SO₂CF₃, —OCF₃, —OCHF₂, C2-C4 alkyl, C2-C4 alkoxy, —CHF₂, —CH₂F, and —CF₃.

In other embodiments, R⁸, R⁹, and R¹⁰ are, independently, selected from F, Cl, Br, Me, OMe, and CF₃.

In further embodiments, m is 2 and n is either 1 or 2.

In certain embodiments, p is 0.

In other embodiments, n is 2.

In still other embodiments, R⁴ and R⁵ taken together form a non-aromatic 5-6 membered ring, which can optionally include a heteroatom selected from N, O and S as a ring member.

In certain embodiments, R⁴ and R⁵ taken together form a cyclohexane or cyclopentane ring.

In some embodiments, the compound has a structure according to the following formula,

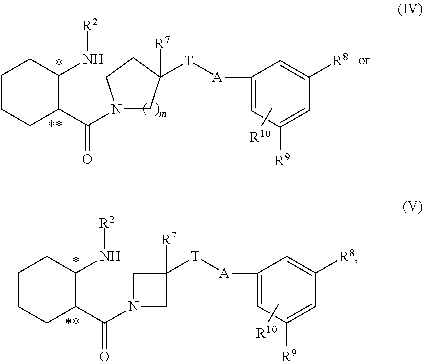

where
m is 1 or 2; -T-A- is —CH₂CONH—, —CH₂NHCO—, —CH₂NHCONH—, —CH₂OCONH—, —NHCONH—, or —OCONH—; R² is H or optionally substituted C1-C6 alkyl; R⁷ is H, OH, or optionally substituted C1-C6 alkyl; R⁸ is halogen or optionally substituted C1-C6 alkyl; and R⁹ and R¹⁰ are, independently, selected from H, halogen, and optionally substituted C1-C6 alkyl.

In certain embodiments, T-A- is —CH₂CONH—.

In other embodiments, -T-A- is —CH₂NHCONH— or —NHCONH—.

In some embodiments, the compound has a structure according to Formula (III) and m is 1.

In other embodiments, the compound has a structure according to Formula (III) and m is 2.

In certain embodiments, R⁸ is F, Cl, Br, or CF₃.

In other embodiments, R⁹ is H, F, Cl, Br, or CF₃.

In still other embodiments, R⁸ and R⁹ are both CF₃, or R⁸ and R⁹ are both F.

In certain embodiments, R⁷ is H.

In some embodiments, the compound has a structure according to the following formula,

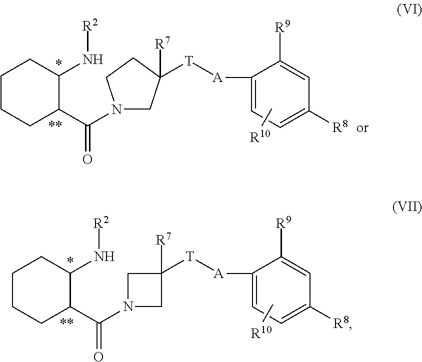

where
m is 1 or 2; -T-A- is —CH₂CONH—, —CH₂NHCO—, —CH₂NHCONH—, —CH₂OCONH—, —NHCONH—, or —OCONH—; R² is H or optionally substituted C1-C6 alkyl; R⁷ is H, OH, or optionally substituted C1-C6 alkyl; R⁸ is halogen, optionally substituted C1-C6 alkyl, optionally substituted C1-C6 alkoxy, or SO₂(optionally substituted C1-C6 alkyl); and R⁹ and R¹⁰ are, independently, selected from H, halogen, and optionally substituted C1-C6 alkyl.

In other embodiments, -T-A- is —CH₂CONH—.

In still other embodiments, -T-A- is —CH₂NHCONH— or —NHCONH—.

In certain embodiments, the compound has a structure according to Formula (V) and m is 1.

In other embodiments, the compound has a structure according to Formula (V) and m is 2.

In some embodiments, R⁸ is F, Br, CF₃, OCF₃, SO₂CF₃, or SO₂CH₃.

In other embodiments, R⁹ is H, F, Cl, Br, or CF₃.

In still other embodiments, R⁷ is H.

In further embodiments, R¹⁰ is H.

In some embodiments, R² is H.

In certain embodiments, the compound has a structure according to

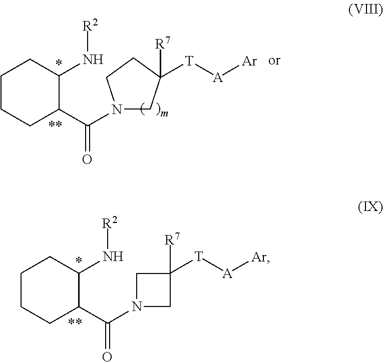

where m is 1 or 2; -T-A- is —CH₂CONH—, —CH₂NHCO—, —CH₂NHCONH—, —CH₂OCONH—, —NHCONH—, or —OCONH—; R² is H or optionally substituted C1-C6 alkyl; R⁷ is H, OH, or optionally substituted C1-C6 alkyl;

### Ar is

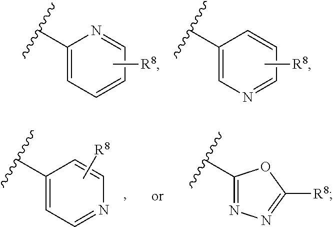

and R⁸ is halogen, optionally substituted C1-C6 alkyl, optionally substituted C1-C6 alkoxy, or SO₂(optionally substituted C1-C6 alkyl).

In some embodiments, -T-A- is —CH₂CONH—.

In other embodiments, R₈ is CF₃.

In certain embodiments, the carbon marked with * has the R configuration. In other embodiments, the carbon marked with * has the S configuration. In further embodiments, the carbon marked with ** has the R configuration. In other embodiments, the carbon marked with ** has the S configuration.

In still other embodiments, the compound has a structure according to the following formula,

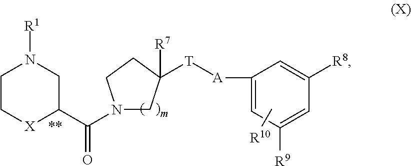

where X is CH₂ or O; m is 1 or 2; -T-A- is —CH₂CONH—, —CH₂NHCO—, —CH₂NHCONH—, —CH₂OCONH—, —NHCONH—, or —OCONH—; R¹ is H or optionally substituted C1-C6 alkyl; R² is H or optionally substituted C1-C6 alkyl; R⁷ is H, OH, or optionally substituted C1-C6 alkyl; R⁸ is halogen, optionally substituted C1-C6 alkyl, optionally substituted C1-C6 alkoxy, or SO₂(optionally substituted C1-C6 alkyl); and R⁹ and R¹⁰ are, independently, selected from H, halogen, and optionally substituted C1-C6 alkyl.

In some embodiments, X is CH₂.

In other embodiments, R¹ is H or unsubstituted C1-C6 alkyl.

In still other embodiments, X is O.

In certain embodiments, R¹ is H.

In some embodiments, m is 2.

In other embodiments, R⁸ and R⁹ are both CF₃.

In other embodiments, the compound has a structure according to the following formula,

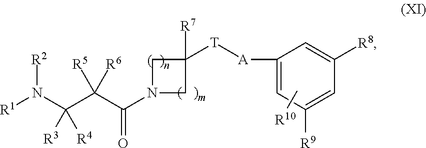

where each of R¹-R⁶ is selected, independently, from H or unsubstituted C1-C6 alkyl; n is 1 or 2; m is 1 or 2; -T-A- is —CH₂CONH—, —CH₂NHCO—, —CH₂NHCONH—, —CH₂OCONH—, —NHCONH—, or —OCONH—; R⁷ is H, OH, or optionally substituted C1-C6 alkyl; R⁸ is halogen, optionally substituted C1-C6 alkyl, optionally substituted C1-C6 alkoxy, or SO₂(optionally substituted C1-C6 alkyl); and R⁹ and R¹⁰ are, independently, selected from H, halogen, and optionally substituted C1-C6 alkyl.

In some embodiments, R¹ and R² are both H.

In other embodiments, R³ and R⁴ are both CH₃.

In certain embodiments, R⁵ and R⁶ are both CH₃.

In still other embodiments, n and m are both 1.

In certain embodiments, n and m are both 2.

In other embodiments, one of n and m is 1, and the other is 2.

In further embodiments, R⁸ and R⁹ are both CF₃.

In certain embodiments, the compound has a structure according to the following formula,

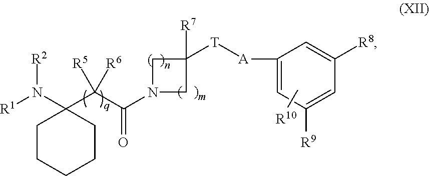

where each of R¹, R², R⁵, R⁶, and R⁷ is selected, independently, from H or unsubstituted C1-C6 alkyl; n is 1 or 2; m is 1 or 2; -T-A- is —CH₂CONH—, —CH₂NHCO—, —CH₂NHCONH—, —CH₂OCONH—, —NHCONH—, or —OCONH—; R⁸ is halogen, optionally substituted C1-C6 alkyl, optionally substituted C1-C6 alkoxy, or SO₂(optionally substituted C1-C6 alkyl); and R⁹ and R¹⁰ are, independently, selected from H, halogen, and optionally substituted C1-C6 alkyl.

In some embodiments, q is 0. In other embodiments, q is 1.

In certain embodiments, R⁵ and R⁶ are both H.

In other embodiments, R¹ and R² are both H.

In still other embodiments, R⁸ and R⁹ are both CF₃.

In other embodiments, the compound has a structure according to the following formula,

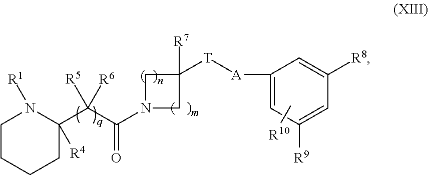

where each of R¹, R⁴, R⁵, R⁶, and R⁷ is selected, independently, from H or unsubstituted C1-C6 alkyl; n is 1 or 2; m is 1 or 2; -T-A- is —CH₂CONH—, —CH₂NHCO—, —CH₂NHCONH—, —CH₂OCONH—, —NHCONH—, or —OCONH—; R⁸ is halogen, optionally substituted C1-C6 alkyl, optionally substituted C1-C6 alkoxy, or SO₂(optionally substituted C1-C6 alkyl); and R⁹ and R¹⁰ are, independently, selected from H, halogen, and optionally substituted C1-C6 alkyl.

In some embodiments, q is 0. In other embodiments, q is 1.

In certain embodiments, R¹ and R⁴ are, independently H or CH₃.

In other embodiments, one of n and m is 1, and the other is 2.

In further embodiments, R⁸ and R⁹ are both CF₃.

In some embodiments, the compound is selected from the group consisting of the following structures:

| No. | Structure |
|---|---|
| 1 | 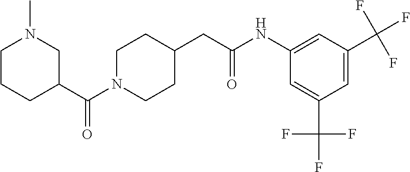 |
| 2 | 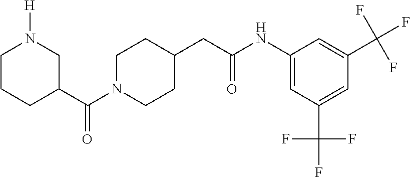 |
| 3 | 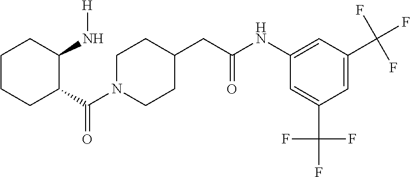 |
| 4 | 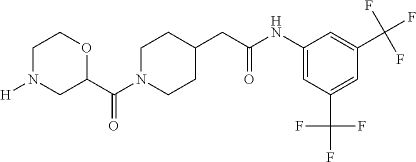 |
| 5 | 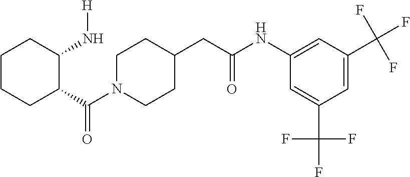 |
| 6 | 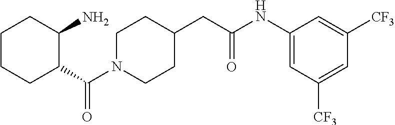 |
| 7 | 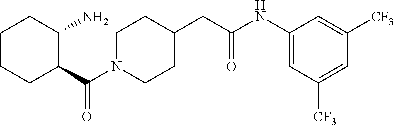 |
| 8 | 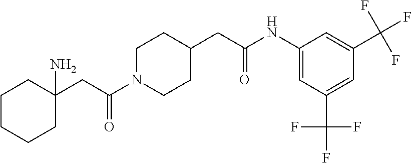 |
| 9 | 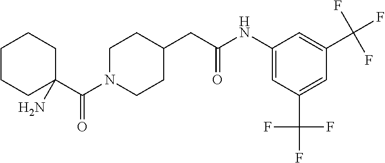 |
| 10 | 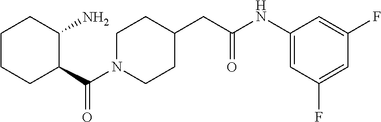 |
| 11 | 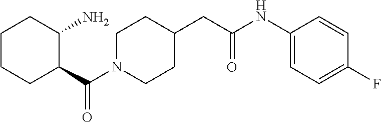 |
| 12 | 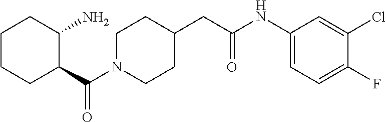 |
| 13 | 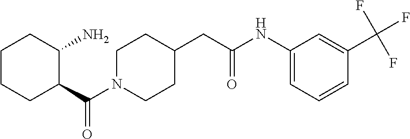 |
| 14 | 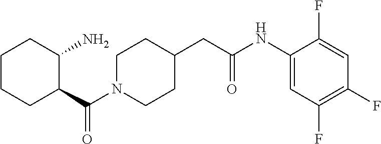 |
| 15 | 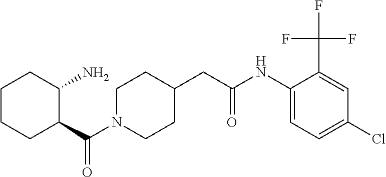 |
| 16 | 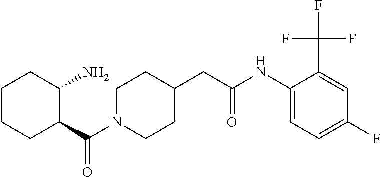 |
| 17 | 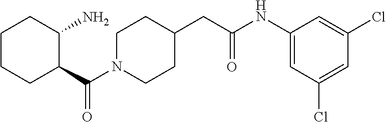 |
| 18 | 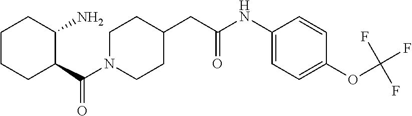 |
| 19 | 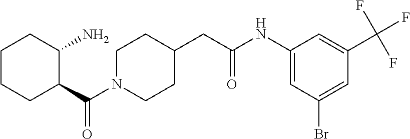 |
| 20 | 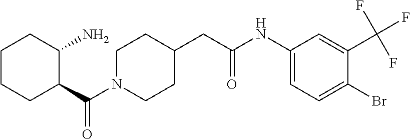 |
| 21 | 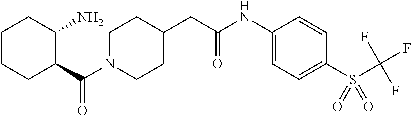 |
| 22 | 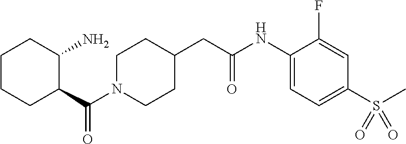 |
| 23 | 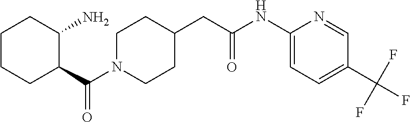 |
| 24 | 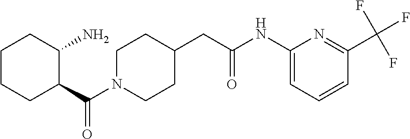 |
| 25 | 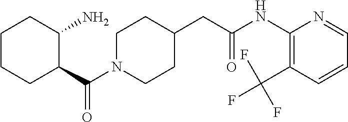 |
| 26 | 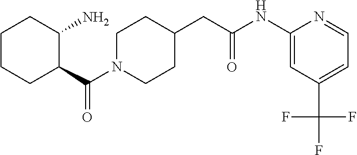 |
| 27 | 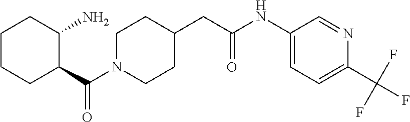 |
| 28 | 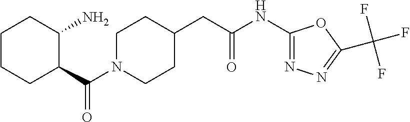 |
| 29 | 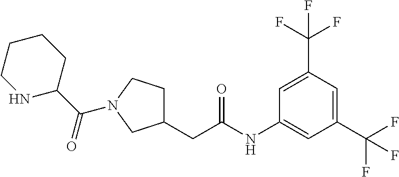 |
| 30 | 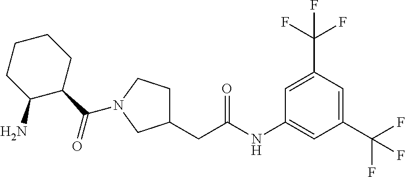 |
| 31 | 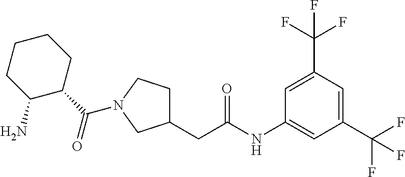 |
| 32 | 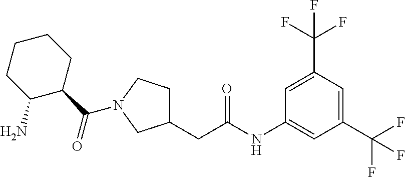 |
| 33 | 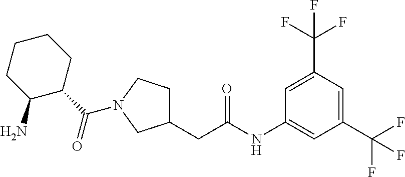 |
| 34 | 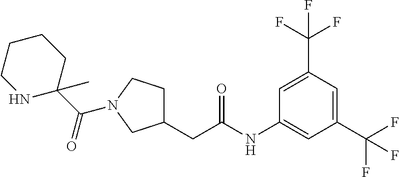 |
| 35 | 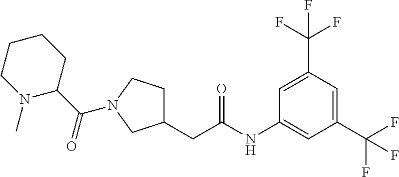 |
| 36 | 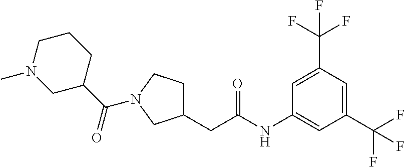 |
| 37 | 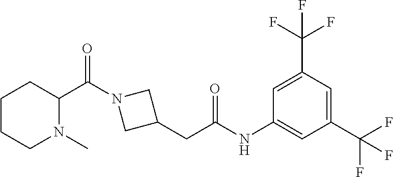 |
| 38 | 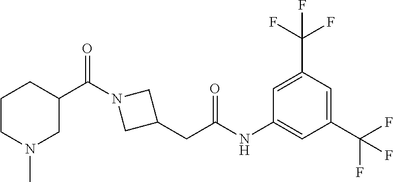 |
| 39 | 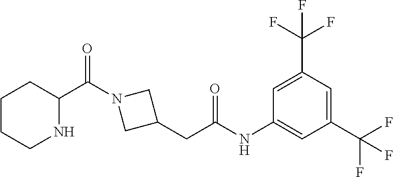 |
| 40 | 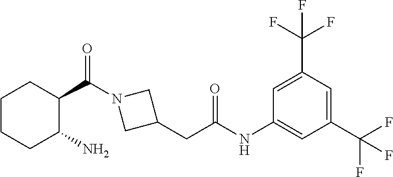 |
| 41 | 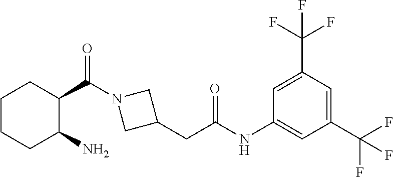 |
| 42 | 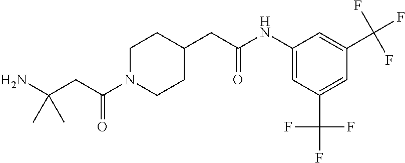 |
| 43 | 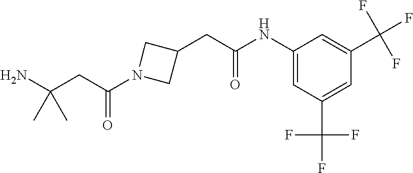 |
| 44 | 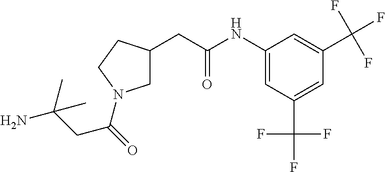 |
| 45 | 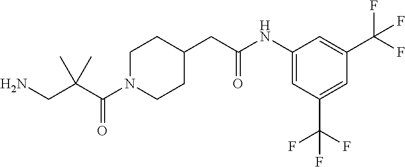 |
| 46 | 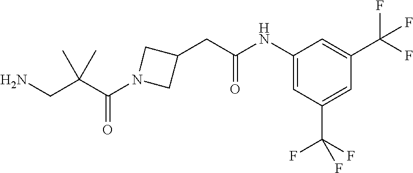 |
| 47 | 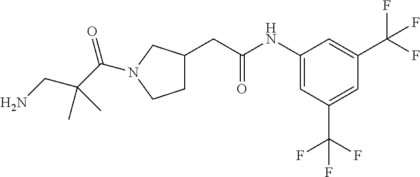 |
| 48 | 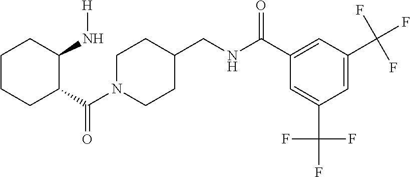 |
| 49 | 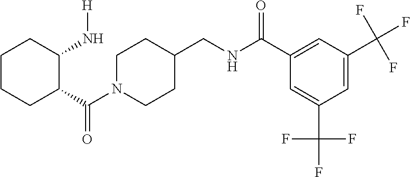 |
| 50 | 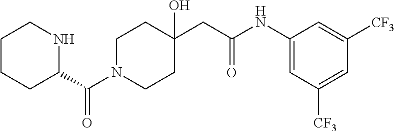 |
| 51 | 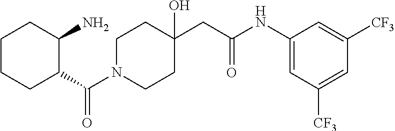 |
| 52 | 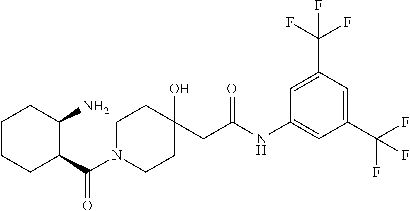 |
| 53 | 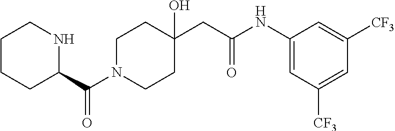 |
| 54 | 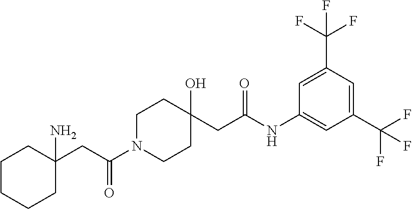 |
| 55 | 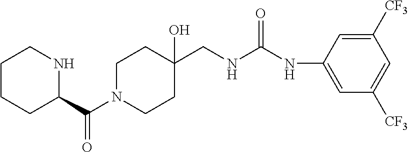 |
| 56 | 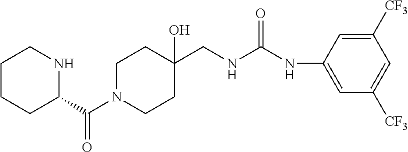 |
| 57 | 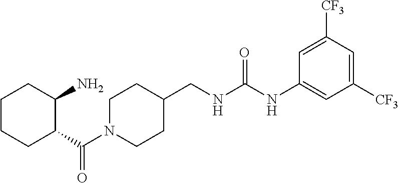 |
| 58 | 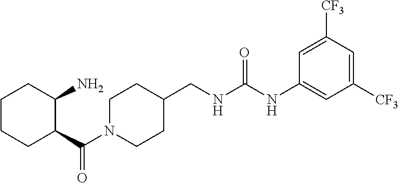 |
| 59 | 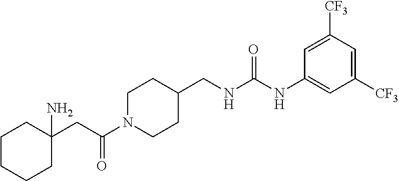 |
| 60 | 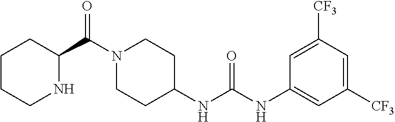 |
| 61 | 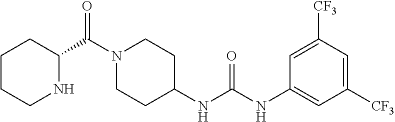 |
| 62 | 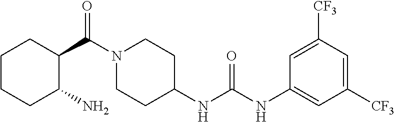 |
| 63 | 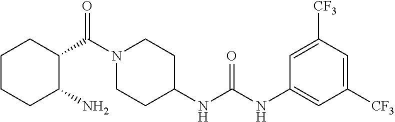 |
| 64 | 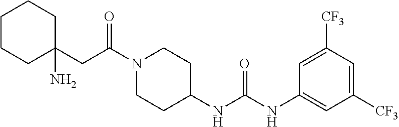 |
| 65 | 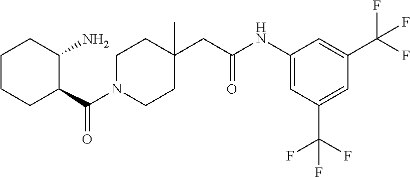 |
| 66 | 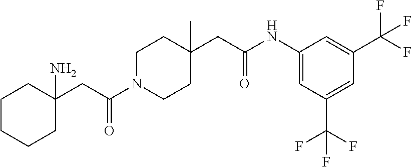 |
| 67 | 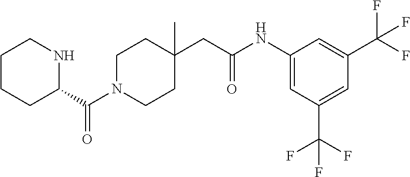 |
| 68 | 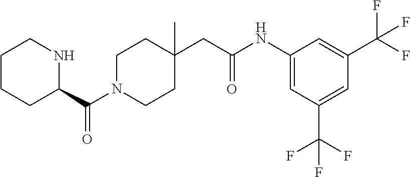 |
| 69 | 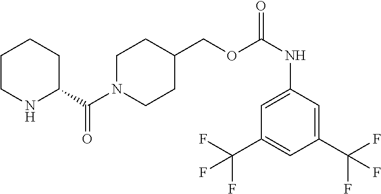 |
| 70 | 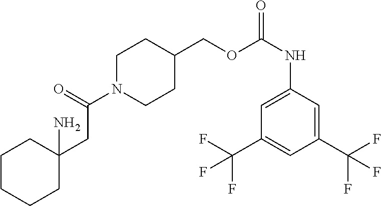 |
| 71 | 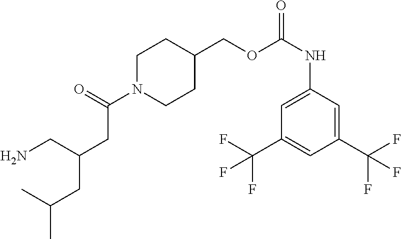 |
| 72 | 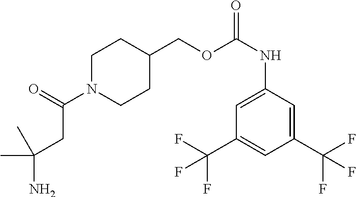 |
| 73 | 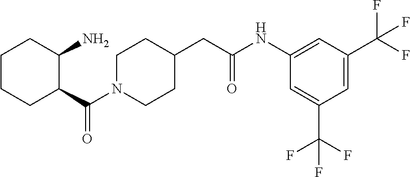 |
| 74 | 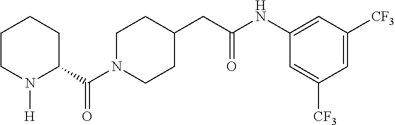 |
| 75 | 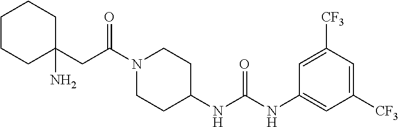 |

In further embodiments, the compound is selected from the following group of structures:

| No. | Structure |
|---|---|
| 1 | 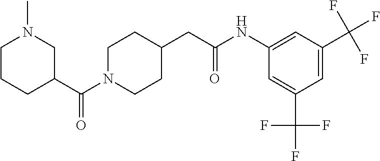 |
| 2 | 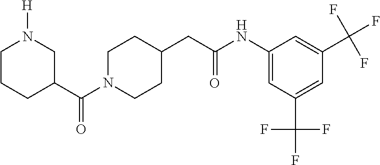 |
| 3 | 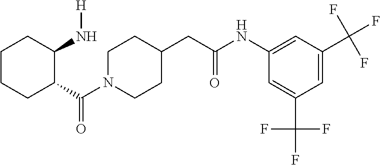 |
| 4 | 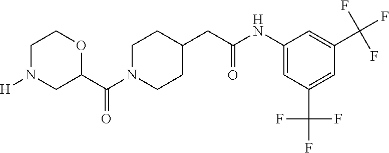 |
| 5 | 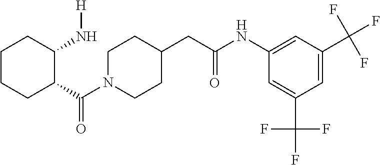 |
| 6 | 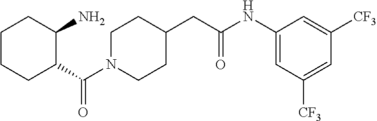 |
| 7 | 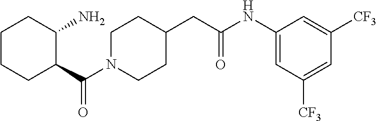 |
| 8 | 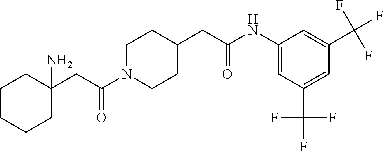 |
| 10 | 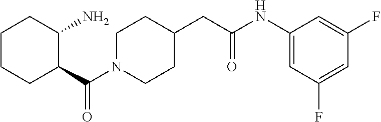 |
| 11 | 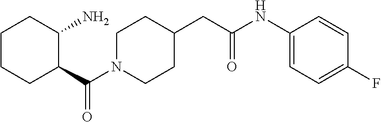 |
| 12 | 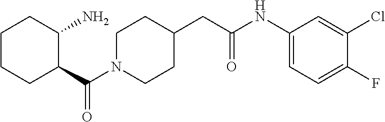 |
| 13 | 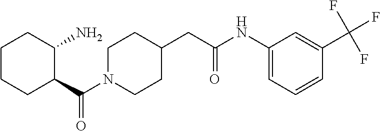 |
| 14 | 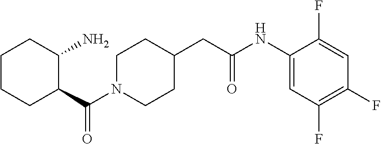 |
| 15 | 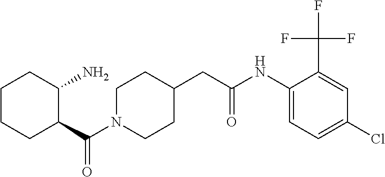 |
| 16 | 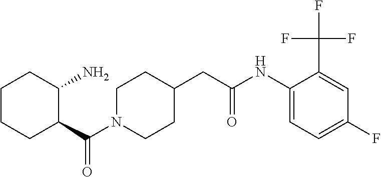 |
| 17 | 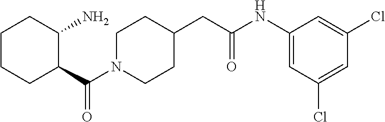 |
| 18 | 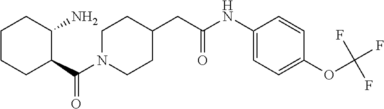 |
| 19 | 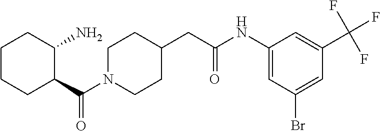 |
| 20 | 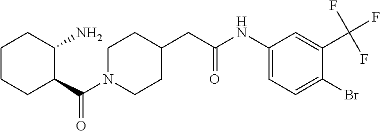 |
| 21 | 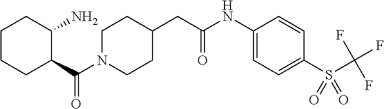 |
| 22 | 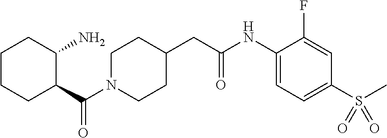 |
| 30 | 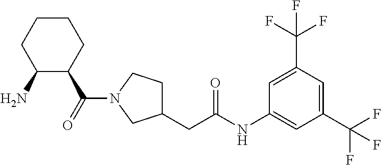 |
| 31 | 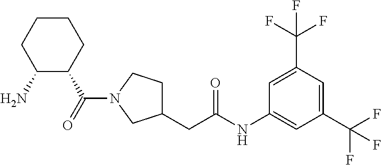 |
| 32 | 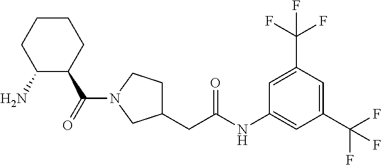 |
| 33 | 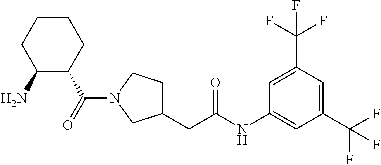 |
| 36 | 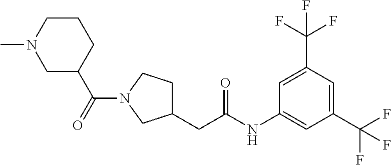 |
| 38 | 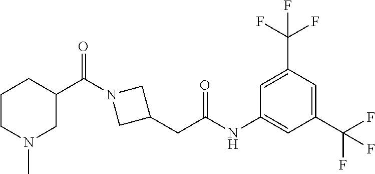 |
| 40 | 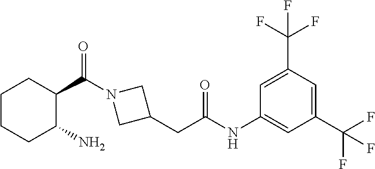 |
| 41 | 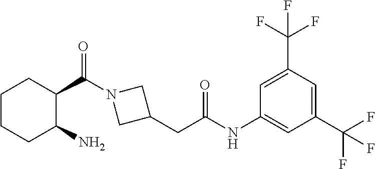 |
| 42 | 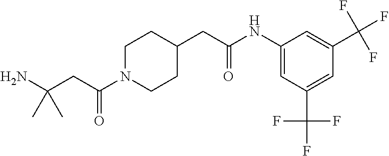 |
| 43 | 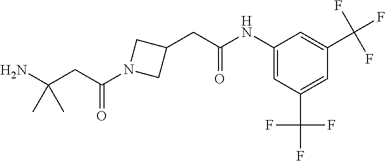 |
| 44 | 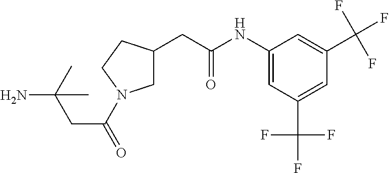 |
| 45 | 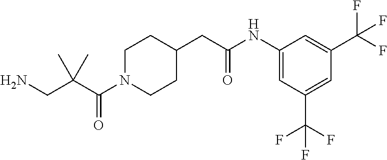 |
| 46 | 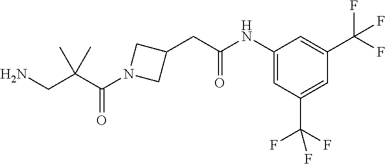 |
| 47 | 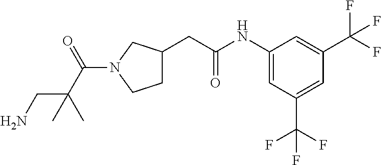 |
| 48 | 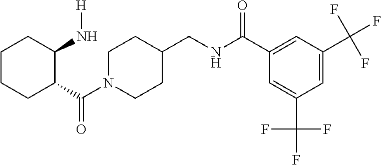 |
| 49 | 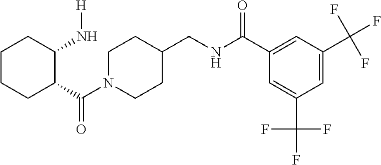 |
| 51 | 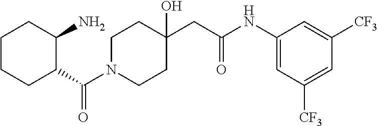 |
| 52 | 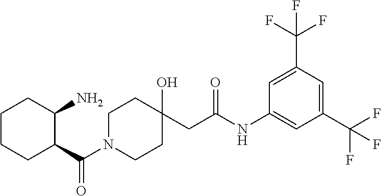 |
| 54 | 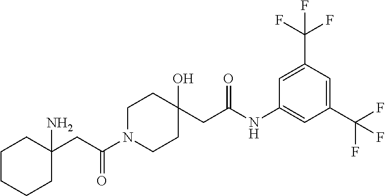 |
| 57 | 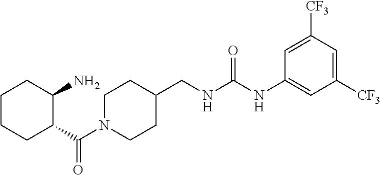 |
| 58 | 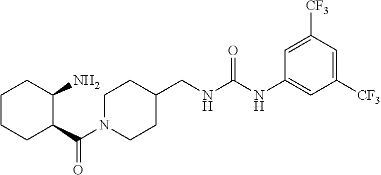 |
| 59 | 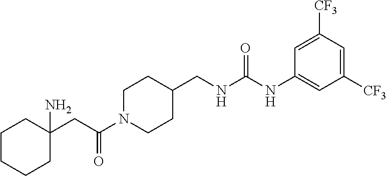 |

In some embodiments, the compound is

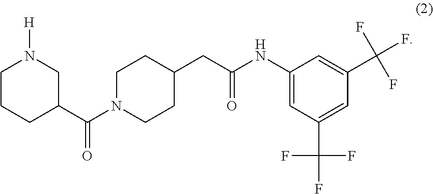

In another aspect, the invention features a pharmaceutical composition that includes any of the compounds described herein, or a pharmaceutically acceptable salt, solvate, or prodrug thereof, or a stereoisomer thereof, or a conjugate thereof, and a pharmaceutically acceptable carrier or excipient.

In some embodiments, the pharmaceutical composition is formulated in unit dosage form.

In further embodiments, the unit dosage form is a tablet, caplet, capsule, lozenge, film, strip, gelcap, or syrup.

In still another aspect, the invention features a method to treat a condition modulated by calcium channel activity, where the method includes administering to a subject in need of such treatment an effective amount of any of the compounds described herein, or a pharmaceutically acceptable salt, solvate, or prodrug thereof, or a stereoisomer thereof, or a conjugate thereof, or a pharmaceutical composition thereof.

In some embodiments, the calcium channel is a T-type calcium channel. In other embodiments, the calcium channel is the Ca_{V} 3.1, Ca_{V} 3.2, or Ca_{V} 3.3 channel.

In certain embodiments, the condition is pain, epilepsy, Parkinson's disease, depression, psychosis (e.g., schizophrenia), or tinnitus.

In some embodiments, the condition is pain or epilepsy.

In certain embodiments, the pain is inflammatory pain or neuropathic pain. In other embodiments, the pain is chronic pain (e.g., peripheral neuropathic pain, central neuropathic pain, musculoskeletal pain, headache, visceral pain, or mixed pain). In further embodiments, the peripheral neuropathic pain is post-herpetic neuralgia, diabetic neuropathic pain, neuropathic cancer pain, failed back-surgery syndrome, trigeminal neuralgia, or phantom limb pain; the central neuropathic pain is multiple sclerosis related pain, Parkinson disease related pain, post-stroke pain, post-traumatic spinal cord injury pain, or pain in dementia; the musculoskeletal pain is osteoarthritic pain and fibromyalgia syndrome; inflammatory pain such as rheumatoid arthritis, or endometriosis; the headache is migraine, cluster headache, tension headache syndrome, facial pain, or headache caused by other diseases; the visceral pain is interstitial cystitis, irritable bowel syndrome, or chronic pelvic pain syndrome; or the mixed pain is lower back pain, neck and shoulder pain, burning mouth syndrome, or complex regional pain syndrome.

In some embodiments, the headache is migraine.

In other embodiments, the pain is acute pain (e.g., nociceptive pain or post-operative pain). In certain embodiments, the acute pain is post-operative pain.

As used herein, the term “alkyl,” “alkenyl” and “alkynyl” include straight-chain, branched-chain and cyclic monovalent substituents, as well as combinations of these, containing only C and H when unsubstituted. Examples include methyl, ethyl, isobutyl, cyclohexyl, cyclopentylethyl, 2-propenyl, 3-butynyl, and the like. Typically, the alkyl, alkenyl and alkynyl groups contain 1-10C (alkyl) or 2-10C (alkenyl or alkynyl). In some embodiments, they contain 1-8C, 1-6C, 1-4C, 1-3C or 1-2C (alkyl); or 2-8C, 2-6C, 2-4C or 2-3C (alkenyl or alkynyl). Further, any hydrogen atom on one of these groups can be replaced with a halogen atom, and in particular a fluoro or chloro, and still be within the scope of the definition of alkyl, alkenyl and alkynyl. For example, CF₃ is a 1C alkyl. These groups may be also be substituted by other substituents as described herein.

Heteroalkyl, heteroalkenyl and heteroalkynyl are similarly defined and contain at least one carbon atom but also contain one or more O, S or N heteroatoms or combinations thereof within the backbone residue whereby each heteroatom in the heteroalkyl, heteroalkenyl or heteroalkynyl group replaces one carbon atom of the alkyl, alkenyl or alkynyl group to which the heteroform corresponds. In some embodiments, the heteroalkyl, heteroalkenyl and heteroalkynyl groups have C at each terminus to which the group is attached to other groups, and the heteroatom(s) present are not located at a terminal position. As is understood in the art, these heteroforms do not contain more than three contiguous heteroatoms. In some embodiments, the heteroatom is O or N.

The designated number of carbons in heteroforms of alkyl, alkenyl and alkynyl includes the heteroatom count. For example, if heteroalkyl is defined as 1-6C, it will contain 1-6 C, N, O, or S atoms such that the heteroalkyl contains at least one C atom and at least one heteroatom, for example 1-5C and 1N or 1-4C and 2N. Similarly, when heteroalkyl is defined as 1-6C or 1-4C, it would contain 1-5C or 1-3C respectively, i.e., at least one C is replaced by O, N or S. Accordingly, when heteroalkenyl or heteroalkynyl is defined as 2-6C (or 2-4C), it would contain 2-6 or 2-4 C, N, O, or S atoms, since the heteroalkenyl or heteroalkynyl contains at least one carbon atom and at least one heteroatom, e.g. 2-5C and 1N or 2-4C and 2O. Further, heteroalkyl, heteroalkenyl or heteroalkynyl substituents may also contain one or more carbonyl groups. Examples of heteroalkyl, heteroalkenyl and heteroalkynyl groups include CH₂OCH₃, CH₂N(CH₃)₂, CH₂OH, (CH₂)ₙNR₂, OR, COOR, CONR₂, (CH₂)ₙ OR, (CH₂)ₙ COR, (CH₂)ₙCOOR, (CH₂)ₙSR, (CH₂)ₙSOR, (CH₂)ₙSO₂R, (CH₂)ₙCONR₂, NRCOR, NRCOOR, OCONR₂, OCOR and the like wherein the R group contains at least one C and the size of the substituent is consistent with the definition of e.g., alkyl, alkenyl, and alkynyl, as described herein.

As used herein, the terms “alkylene,” “alkenylene” and “alkynylene” refers to divalent or trivalent groups having a specified size, typically 1-2C, 1-3C, 1-4C, 1-6C or 1-8C for the saturated groups and 2-3C, 2-4C, 2-6C or 2-8C for the unsaturated groups. They include straight-chain, branched-chain and cyclic forms as well as combinations of these, containing only C and H when unsubstituted. Because they are divalent, they can link together two parts of a molecule, as exemplified by X in the compounds described herein. Examples are methylene, ethylene, propylene, cyclopropan-1,1-diyl, ethylidene, 2-butene-1,4-diyl, and the like. These groups can be substituted by the groups typically suitable as substituents for alkyl, alkenyl and alkynyl groups as set forth herein. Thus C═O is a C1 alkylene that is substituted by ═O, for example.

Heteroalkylene, heteroalkenylene and heteroalkynylene are similarly defined as divalent groups having a specified size, typically 1-3C, 1-4C, 1-6C or 1-8C for the saturated groups and 2-3C, 2-4C, 2-6C or 2-8C for the unsaturated groups. They include straight chain, branched chain and cyclic groups as well as combinations of these, and they further contain at least one carbon atom but also contain one or more O, S or N heteroatoms or combinations thereof within the backbone residue, whereby each heteroatom in the heteroalkylene, heteroalkenylene or heteroalkynylene group replaces one carbon atom of the alkylene, alkenylene or alkynylene group to which the heteroform corresponds. As is understood in the art, these heteroforms do not contain more than three contiguous heteroatoms.

“Aromatic” moiety or “aryl” moiety refers to any monocyclic or fused ring bicyclic system which has the characteristics of aromaticity in terms of electron distribution throughout the ring system and includes a monocyclic or fused bicyclic moiety such as phenyl or naphthyl; “heteroaromatic” or “heteroaryl” also refers to such monocyclic or fused bicyclic ring systems containing one or more heteroatoms selected from O, S and N. The inclusion of a heteroatom permits inclusion of 5-membered rings to be considered aromatic as well as 6-membered rings. Thus, typical aromatic/heteroaromatic systems include pyridyl, pyrimidyl, indolyl, benzimidazolyl, benzotriazolyl, isoquinolyl, quinolyl, benzothiazolyl, benzofuranyl, thienyl, furyl, pyrrolyl, thiazolyl, oxazolyl, isoxazolyl, benzoxazolyl, benzoisoxazolyl, imidazolyl and the like. Because tautomers are theoretically possible, phthalimido is also considered aromatic. Typically, the ring systems contain 5-12 ring member atoms or 6-10 ring member atoms. In some embodiments, the aromatic or heteroaromatic moiety is a 6-membered aromatic rings system optionally containing 1-2 nitrogen atoms. More particularly, the moiety is an optionally substituted phenyl, pyridyl, indolyl, pyrimidyl, pyridazinyl, benzothiazolyl or benzimidazolyl, pyrazolyl, imidazolyl, isoxazolyl, thiazolyl, benzothiazolyl, indolyl. Even more particularly, such moiety is phenyl, pyridyl, or pyrimidyl and even more particularly, it is phenyl.

“O-aryl” or “O-heteroaryl” refers to aromatic or heteroaromatic systems which are coupled to another residue through an oxygen atom. A typical example of an O-aryl is phenoxy. Similarly, “arylalkyl” refers to aromatic and heteroaromatic systems which are coupled to another residue through a carbon chain, saturated or unsaturated, typically of 1-8C, 1-6C or more particularly 1-4C or 1-3C when saturated or 2-8C, 2-6C, 2-4C or 2-3C when unsaturated, including the heteroforms thereof. For greater certainty, arylalkyl thus includes an aryl or heteroaryl group as defined above connected to an alkyl, heteroalkyl, alkenyl, heteroalkenyl, alkynyl or heteroalkynyl moiety also as defined above. Typical arylalkyls would be an aryl(6-12C)alkyl(1-8C), aryl(6-12C)alkenyl(2-8C), or aryl(6-12C)alkynyl(2-8C), plus the heteroforms. A typical example is phenylmethyl, commonly referred to as benzyl.

Typical optional substituents on aromatic or heteroaromatic groups include independently halo, CN, NO₂, CF₃, OCF₃, COOR′, CONR′₂, OR′, SR′, SOR′, SO₂R′, NR′₂, NR′(CO)R′, NR′C(O)OR′, NR′C(O)NR′₂, NR′SO₂NR′₂, or NR′SO₂R′, wherein each R′ is independently H or an optionally substituted group selected from alkyl, alkenyl, alkynyl, heteroalkyl, heteroalkenyl, heteroalkynyl, heteroaryl, and aryl (all as defined above); or the substituent may be an optionally substituted group selected from alkyl, alkenyl, alkynyl, heteroalkyl, heteroalkenyl, heteroalkynyl, aryl, heteroaryl, O-aryl, O-heteroaryl and arylalkyl.

Optional substituents on a non-aromatic group (e.g., alkyl, alkenyl, and alkynyl groups), are typically selected from the same list of substituents suitable for aromatic or heteroaromatic groups and may further be selected from ═O and ═NOR′ where R′ is H or an optionally substituted group selected from alkyl, alkenyl, alkynyl, heteroalkyl, heteroalkenyl, heteralkynyl, heteroaryl, and aryl (all as defined above).

Halo may be any halogen atom, especially F, Cl, Br, or I, and more particularly it is fluoro or chloro.

In general, a substituent group (e.g., alkyl, alkenyl, alkynyl, or aryl (including all heteroforms defined above) may itself optionally be substituted by additional substituents. The nature of these substituents is similar to those recited with regard to the substituents on the basic structures above. Thus, where an embodiment of a substituent is alkyl, this alkyl may optionally be substituted by the remaining substituents listed as substituents where this makes chemical sense, and where this does not undermine the size limit of alkyl per se; e.g., alkyl substituted by alkyl or by alkenyl would simply extend the upper limit of carbon atoms for these embodiments, and is not included. However, alkyl substituted by aryl, amino, halo and the like would be included. For example, where a group is substituted, the group may be substituted with 1, 2, 3, 4, 5, or 6 substituents. Optional substituents include, but are not limited to: 1C-6C alkyl or heteroaryl, 2C-6C alkenyl or heteroalkenyl, 2C-6C alkynyl or heteroalkynyl, halogen; aryl, heteroaryl, azido (—N₃), nitro (—NO₂), cyano (—CN), acyloxy(—OC(═O)R′), acyl (—C(═O)R′), alkoxy (—OR′), amido (—NR′C(═O)R″ or —C(═O)NRR′), amino (—NRR′), carboxylic acid (—CO₂H), carboxylic ester (—CO₂R′), carbamoyl (—OC(═O)NR′R″ or —NRC(═O)OR′), hydroxy (—OH), isocyano (—NC), sulfonate (—S(═O)₂OR), sulfonamide (—S(═O)₂NRR′ or —NRS(═O)₂R′), or sulfonyl (—S(═O)₂R), where each R or R′ is selected, independently, from H, 1C-6C alkyl or heteroaryl, 2C-6C alkenyl or heteroalkenyl, 2C-6C alkynyl or heteroalkynyl, aryl, or heteroaryl. A substituted group may have, for example, 1, 2, 3, 4, 5, 6, 7, 8, or 9 substituents.

The term an “effective amount” of an agent (e.g., a compound according to any of Formulas (I)-(XIII) or any of Compounds 1-75), as used herein, is that amount sufficient to effect beneficial or desired results, such as clinical results, and, as such, an “effective amount” depends upon the context in which it is being applied. For example, in the context of administering an agent that is a modulator of a calcium channel (e.g., Ca_{V} 3.1, Ca_{V} 3.2, or Ca_{V} 3.3), an effective amount of an agent is, for example, an amount sufficient to achieve a change in calcium channel activity as compared to the response obtained without administration of the agent.

The term “pharmaceutical composition,” as used herein, represents a composition containing a compound described herein (e.g., a compound according to any of Formulas (I)-(XIII) or any of Compounds 1-75 in Table 1) formulated with a pharmaceutically acceptable excipient. In some embodiments, the pharmaceutical composition is manufactured or sold with the approval of a governmental regulatory agency as part of a therapeutic regimen for the treatment of disease in a mammal. Pharmaceutical compositions can be formulated, for example, for oral administration in unit dosage form (e.g., a tablet, capsule, caplet, gelcap, or syrup); for topical administration (e.g., as a cream, gel, lotion, or ointment); for intravenous administration (e.g., as a sterile solution free of particulate emboli and in a solvent system suitable for intravenous use); or in any other formulation described herein.

A “pharmaceutically acceptable excipient,” as used herein, refers any ingredient other than the compounds described herein (for example, a vehicle capable of suspending or dissolving the active compound) and having the properties of being nontoxic and non-inflammatory in a patient. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspensing or dispersing agents, sweeteners, or waters of hydration. Exemplary excipients include, but are not limited to: butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, stearic acid, sucrose, talc, titanium dioxide, vitamin A, vitamin E, vitamin C, and xylitol.

The term “pharmaceutically acceptable prodrugs” as used herein, represents those prodrugs of the compounds of the present invention that are, within the scope of sound medical judgment, suitable for use in contact with the tissues of humans and animals with undue toxicity, irritation, allergic response, and the like, commensurate with a reasonable benefit/risk ratio, and effective for their intended use, as well as the zwitterionic forms, where possible, of the compounds of the invention.

The term “pharmaceutically acceptable salt,” as use herein, represents those salts of the compounds described here (e.g., a compound according to any of Formulas (I)-(XIII) or any of Compounds 1-75 in Table 1) that are, within the scope of sound medical judgment, suitable for use in contact with the tissues of humans and animals without undue toxicity, irritation, allergic response and the like and are commensurate with a reasonable benefit/risk ratio. Pharmaceutically acceptable salts are well known in the art. For example, pharmaceutically acceptable salts are described in: Berge et al., J. Pharmaceutical Sciences 66:1-19, 1977 and in Pharmaceutical Salts Properties, Selection, and Use, (Eds. P. H. Stahl and C. G. Wermuth), Wiley-VCH, 2008. The salts can be prepared in situ during the final isolation and purification of the compounds described herein or separately by reacting the free base group with a suitable organic acid.

The compounds of the invention may have ionizable groups so as to be capable of preparation as pharmaceutically acceptable salts. These salts may be acid addition salts involving inorganic or organic acids or the salts may, in the case of acidic forms of the compounds of the invention be prepared from inorganic or organic bases. Frequently, the compounds are prepared or used as pharmaceutically acceptable salts prepared as addition products of pharmaceutically acceptable acids or bases. Suitable pharmaceutically acceptable acids and bases are well-known in the art, such as hydrochloric, sulphuric, hydrobromic, acetic, lactic, citric, or tartaric acids for forming acid addition salts, and potassium hydroxide, sodium hydroxide, ammonium hydroxide, caffeine, various amines, and the like for forming basic salts. Methods for preparation of the appropriate salts are well-established in the art.

Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts and the like. Representative alkali or alkaline earth metal salts include sodium, lithium, potassium, calcium, magnesium and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine and the like.

The term “pharmaceutically acceptable solvate” as used herein means a compound as described herein (e.g., a compound according to any of Formulas (I)-(XIII) or any of Compounds 1-75 in Table 1) where molecules of a suitable solvent are incorporated in the crystal lattice. A suitable solvent is physiologically tolerable at the dosage administered. For example, solvates may be prepared by crystallization, recrystallization, or precipitation from a solution that includes organic solvents, water, or a mixture thereof. Examples of suitable solvents are ethanol, water (for example, mono-, di-, and tri-hydrates), N-methylpyrrolidinone (NMP), dimethyl sulfoxide (DMSO), N,N′-dimethylformamide (DMF), N,N′-dimethylacetamide (DMAC), 1,3-dimethyl-2-imidazolidinone (DMEU), 1,3-dimethyl-3,4,5,6-tetrahydro-2-(1H)-pyrimidinone (DMPU), acetonitrile (ACN), propylene glycol, ethyl acetate, benzyl alcohol, 2-pyrrolidone, benzyl benzoate, and the like. When water is the solvent, the molecule is referred to as a “hydrate.”

The term “prevent,” as used herein, refers to prophylactic treatment or treatment that prevents one or more symptoms or conditions of a disease, disorder, or conditions described herein (for example, pain (e.g., chronic or acute pain), epilepsy, Alzheimer's disease, Parkinson's disease, cardiovascular disease, diabetes, cancer, sleep disorders, obesity, psychosis such as schizophrenia, overactive bladder, renal disease, neuroprotection, addiction, and male birth control). Preventative treatment can be initiated, for example, prior to (“pre-exposure prophylaxis”) or following (“post-exposure prophylaxis”) an event that precedes the onset of the disease, disorder, or conditions. Preventive treatment that includes administration of a compound described herein (e.g., a compound according to any of Formulas (I)-(XIII) or any of Compounds 1-75 in Table 1), or a pharmaceutically acceptable salt or solvate thereof, or a pharmaceutical composition thereof, can be acute, short-term, or chronic. The doses administered may be varied during the course of preventative treatment.

The term “prodrug,” as used herein, represents compounds that are rapidly transformed in vivo to the parent compound of the above formula, for example, by hydrolysis in blood. Prodrugs of the compounds described herein may be conventional esters. Some common esters that have been utilized as prodrugs are phenyl esters, aliphatic (C1-C8 or C8-C24) esters, cholesterol esters, acyloxymethyl esters, carbamates, and amino acid esters. For example, a compound that contains an OH group may be acylated at this position in its prodrug form. A thorough discussion is provided in T. Higuchi and V. Stella, Pro-drugs as Novel Delivery Systems, Vol. 14 of the A.C.S. Symposium Series, Edward B. Roche, ed., Bioreversible Carriers in Drug Design, American Pharmaceutical Association and Pergamon Press, 1987, and Judkins et al., Synthetic Communications 26(23):4351-4367, 1996, each of which is incorporated herein by reference. Preferably, prodrugs of the compounds of the present invention are suitable for use in contact with the tissues of humans and animals with undue toxicity, irritation, allergic response, and the like, commensurate with a reasonable benefit/risk ratio, and effective for their intended use.

In addition, the compounds of the invention may be coupled through conjugation to substances designed to alter the pharmacokinetics, for targeting, or for other reasons. Thus, the invention further includes conjugates of these compounds. For example, polyethylene glycol is often coupled to substances to enhance half-life; the compounds may be coupled to liposomes covalently or noncovalently or to other particulate carriers. They may also be coupled to targeting agents such as antibodies or peptidomimetics, often through linker moieties. Thus, the invention is also directed to compounds (e.g., a compound according to any of Formulas (I)-(XIII) or any of Compounds 1-75 in Table 1) when modified so as to be included in a conjugate of this type.

As used herein, and as well understood in the art, “to treat” a condition or “treatment” of the condition (e.g., the conditions described herein such as pain (e.g., chronic or acute pain), epilepsy, Alzheimer's disease, Parkinson's disease, cardiovascular disease, diabetes, cancer, sleep disorders, obesity, psychosis such as schizophrenia, overactive bladder, renal disease, neuroprotection, addiction, and male birth control) is an approach for obtaining beneficial or desired results, such as clinical results. Beneficial or desired results can include, but are not limited to, alleviation or amelioration of one or more symptoms or conditions; diminishment of extent of disease, disorder, or condition; stabilized (i.e., not worsening) state of disease, disorder, or condition; preventing spread of disease, disorder, or condition; delay or slowing the progress of the disease, disorder, or condition; amelioration or palliation of the disease, disorder, or condition; and remission (whether partial or total), whether detectable or undetectable. “Palliating” a disease, disorder, or condition means that the extent and/or undesirable clinical manifestations of the disease, disorder, or condition are lessened and/or time course of the progression is slowed or lengthened, as compared to the extent or time course in the absence of treatment.

The term “unit dosage form” refers to a physically discrete unit suitable as a unitary dosage for human subjects and other mammals, each unit containing a predetermined quantity of active material calculated to produce the desired therapeutic effect, in association with any suitable pharmaceutical excipient or excipients. Exemplary, non-limiting unit dosage forms include a tablet (e.g., a chewable tablet), caplet, capsule (e.g., a hard capsule or a soft capsule), lozenge, film, strip, gelcap, and syrup.

In some cases, the compounds of the invention contain one or more chiral centers. The invention includes each of the isolated stereoisomeric forms as well as mixtures of stereoisomers in varying degrees of chiral purity, including racemic mixtures. It also encompasses the various diastereomers and tautomers that can be formed.

Other features and advantages of the invention will be apparent from the following detailed description, the drawing, and the claims.

### BRIEF DESCRIPTION OF THE DRAWING

FIG. 1 shows separation of a racemic sample of a compound of Formula (I) into two enantiomers using chiral HPLC chromatography.

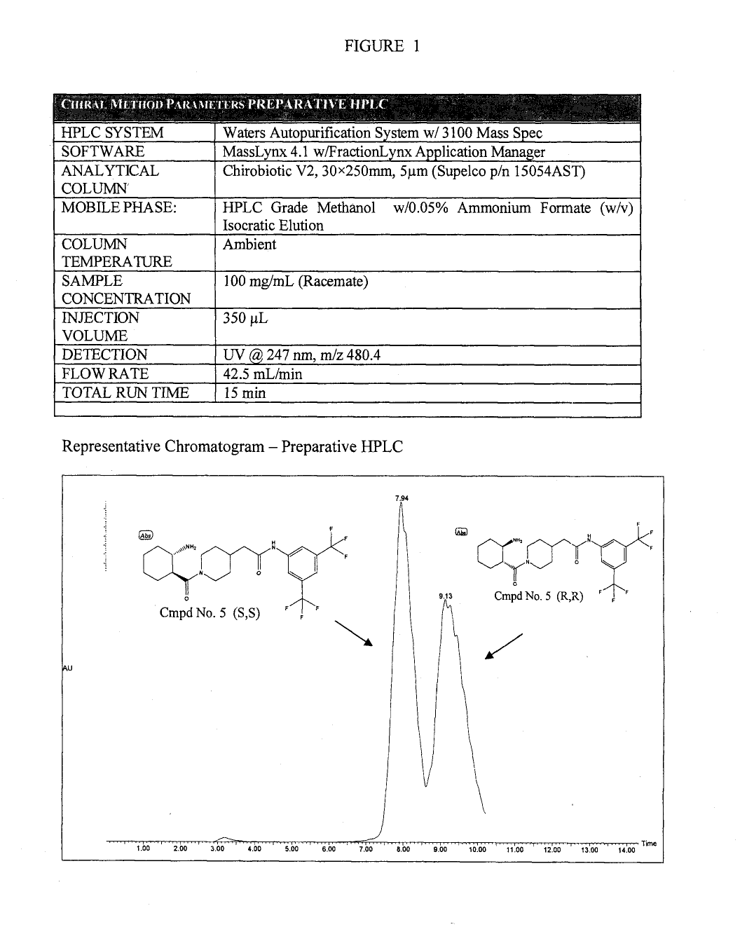

### DETAILED DESCRIPTION OF THE INVENTION

### Compounds

The invention features compounds according to Formula (I),

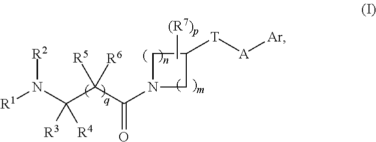

or a pharmaceutically acceptable salt, solvate, or prodrug thereof, or a stereoisomer thereof, or a conjugate thereof, where

Ar is phenyl or a 5-6 membered heteroaryl ring containing at least one heteroatom selected from N, O and S as a ring member, and optionally substituted with at least one R⁸, R⁹, or R¹⁰;

T is CH₂, O, or NR¹;

A is [T]-C(O)—NR¹ or [T]-NR¹—C(O)— or [T]-C(O)—O—, or [T]-O—C(O)—, —NR¹—C(O)—NR¹—; [T]-O—C(O)—NR¹—; or [T]-NR¹—C(O)—O—; where [T] indicates which atom of A is linked to T in Formula (II);

R¹ and R² are independently selected from the group consisting of H, optionally substituted C1-C6 alkyl, C1-C6 optionally substituted alkylsulfonyl, and optionally substituted C1-C6 acyl;

R³, R⁴, R⁵, R⁶ and R⁷ are independently selected from H, optionally substituted C1-C6 alkyl, halo, hydroxy, CN, optionally substituted C1-C6 alkoxy, and optionally substituted C1-C6 heteroalkyl;

where R² and R³, or R³ and R⁴, or R⁴ and R⁵, or R⁶ and R², or two R⁶ if two R⁶ are present (any one of these pairs, but not more than one pair) can optionally be taken together to form a non-aromatic 5-6 membered ring, which can optionally include up to two heteroatoms selected from N, O and S as ring members;

and two R⁷ can optionally be taken together to form a non-aromatic 5-6 membered ring, which can optionally include a heteroatom selected from N, O and S as a ring member

m and n are independently 1 or 2;

p is 0-2;

q is 0, 1 or 2;

R⁸, R⁹, and R¹⁰ are optional substituents that are independently selected from the group consisting of H, halogen, CN, —SO₂—(C1-C4 alkyl), optionally substituted C1-C6 alkyl, optionally substituted C1-C6 alkoxy, optionally substituted aryl, and optionally substituted heteroaryl, provided at least one of R⁸, R⁹, and R¹⁰ is present and is not H.

Other compounds are also described by any of Formulas (II)-(XIII) as described herein:

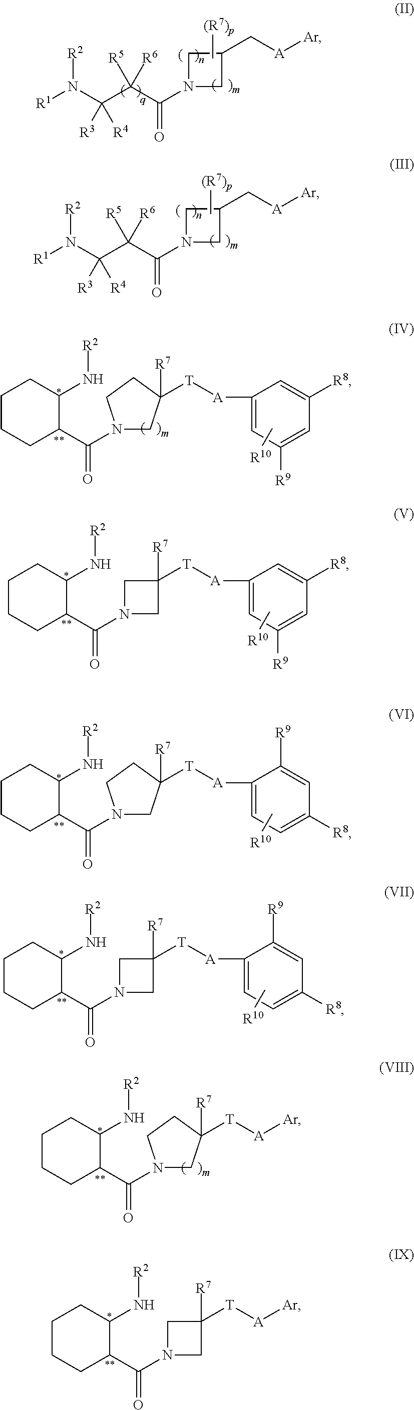

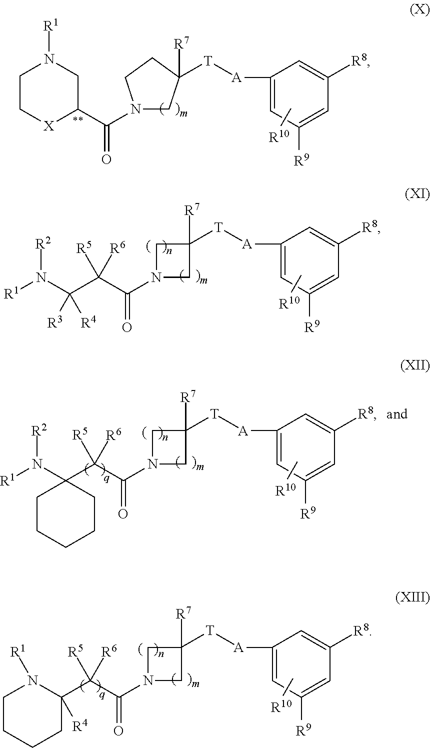

Representative compounds of the invention include Compounds 1-75 of Table 1. Exemplary methods of synthesis and uses of these compounds are also described.

### Utility and Administration

The compounds described herein (e.g., a compound according to any of Formulas (I)-(XIII) or any of Compounds 1-75 in Table 1) are useful in the methods of the invention and, while not bound by theory, are believed to exert their desirable effects through their ability to modulate the activity of calcium channels, particularly the activity of T-type calcium channels. This makes them useful for treatment of certain conditions where modulation of calcium channels (e.g., T-type calcium channels such as Ca_{V} 3.1, Ca_{V} 3.2, or Ca_{V} 3.3), is desired including pain, epilepsy, migraine, Parkinson's disease, depression, schizophrenia, psychosis, and tinnitus.

### Modulation of Calcium Channels

The entry of calcium into cells through voltage-gated calcium channels mediates a wide variety of cellular and physiological responses, including excitation-contraction coupling, hormone secretion and gene expression (e.g., Miller et al., Science 235:46-52 (1987); Augustine et al., Annu Rev Neurosci 10: 633-693 (1987)). In neurons, calcium channels directly affect membrane potential and contribute to electrical properties such as excitability, repetitive firing patterns and pacemaker activity. Calcium entry further affects neuronal functions by directly regulating calcium-dependent ion channels and modulating the activity of calcium-dependent enzymes such as protein kinase C and calmodulin-dependent protein kinase II. An increase in calcium concentration at the presynaptic nerve terminal triggers the release of neurotransmitter, which also affects neurite outgrowth and growth cone migration in developing neurons.

Calcium channels mediate a variety of normal physiological functions, and are also implicated in a number of human disorders as described herein. For example, calcium channels also have been shown to mediate the development and maintenance of the neuronal sensitization and hyperexcitability processes associated with neuropathic pain, and provide attractive targets for the development of analgesic drugs (reviewed in Vanegas et al., Pain 85: 9-18 (2000)). Native calcium channels have been classified by their electrophysiological and pharmacological properties into T-, L-, N-, P/Q- and R-types (reviewed in Catterall, Annu Rev Cell Dev Biol 16: 521-555, 2000; Huguenard, Annu Rev Physiol 58: 329-348, 1996). The L-, N- and P/Q-type channels activate at more positive potentials (high voltage-activated) and display diverse kinetics and voltage-dependent properties (Id.). T-type channels can be distinguished by having a more negative range of activation and inactivation, rapid inactivation, slow deactivation, and smaller single-channel conductances. There are three subtypes of T-type calcium channels that have been molecularly, pharmacologically, and elecrophysiologically identified: these subtypes have been termed α_{1G}, α_{1H}, and α_{1I} (alternately called Ca_{V} 3.1, Ca_{V} 3.2 and Ca_{V} 3.3 respectively).

T-type calcium channels are involved in various medical conditions. In mice lacking the gene expressing the 3.1 subunit, resistance to absence seizures was observed (Kim et al., Mol. Cell Neurosci. 18(2): 235-245 (2001)). Other studies have also implicated the 3.2 subunit in the development of epilepsy (Su et al., J. Neurosci. 22: 3645-3655 (2002)). There is also evidence that some existing anticonvulsant drugs, such as ethosuximide, function through the blockade of T-type channels (Gomora et al., Mol. Pharmacol. 60: 1121-1132 (2001)).

Low voltage-activated calcium channels are highly expressed in tissues of the cardiovascular system. There is also a growing body of evidence that suggests that T-type calcium channels are abnormally expressed in cancerous cells and that blockade of these channels may reduce cell proliferation in addition to inducing apoptosis. Recent studies also show that the expression of T-type calcium channels in breast cancer cells is proliferation state dependent, i.e. the channels are expressed at higher levels during the fast-replication period, and once the cells are in a non-proliferation state, expression of this channel is minimal. Therefore, selectively blocking calcium channel entry into cancerous cells may be a valuable approach for preventing tumor growth (e.g., PCT Patent Publication Nos. WO 05/086971 and WO 05/77082; Taylor et al., World J. Gastroenterol. 14(32): 4984-4991 (2008); Heo et al., Biorganic & Medicinal Chemistry Letters 18:3899-3901 (2008)).

T-type calcium channels may also be involved in still other conditions. A recent study also has shown that T-type calcium channel antagonists inhibit high-fat diet-induced weight gain in mice. In addition, administration of a selective T-type channel antagonist reduced body weight and fat mass while concurrently increasing lean muscle mass (e.g., Uebele et al., The Journal of Clinical Investigation, 119(6):1659-1667 (2009)). T-type calcium channels may also be involved in pain (see for example: US Patent Publication No. 2003/0086980; PCT Publication Nos. WO 03/007953 and WO 04/000311). In addition to cardiovascular disease, epilepsy (see also US Patent Publication No. 2006/0025397), cancer, and chronic or acute pain, T-type calcium channels have been implicated in diabetes (US Patent Publication No. 2003/0125269), sleep disorders (US Patent Publication No. 2006/0003985), Parkinson's disease and psychosis such as schizophrenia (US Patent Publication No. 2003/0087799); overactive bladder (Sui et al., British Journal of Urology International 99(2): 436-441 (2007); US Patent Publication No. 2004/0197825), renal disease (Hayashi et al., Journal of Pharmacological Sciences 99: 221-227 (2005)), anxiety and alcoholism (US Patent Publication No. 2009/0126031), neuroprotection, and male birth control.

The modulation of ion channels by the compounds described herein (e.g., a compound according to any of Formulas (I)-(XIII) or any of Compounds 1-75 in Table 1) can be measured according to methods known in the art (e.g., in the references provided herein). Modulators of ion channels, e.g., voltage gated calcium ion channels, and the medicinal chemistry or methods by which such compounds can be identified, are also described in, for example: Birch et al., Drug Discovery Today, 9(9):410-418 (2004); Audesirk, “Chapter 6—Electrophysiological Analysis of Ion Channel Function,” Neurotoxicology: Approaches and Methods, 137-156 (1995); Camerino et al., “Chapter 4: Therapeutic Approaches to Ion Channel Diseases,” Advances in Genetics, 64:81-145 (2008); Petkov, “Chapter 16—Ion Channels,” Pharmacology: Principles and Practice, 387-427 (2009); Standen et al., “Chapter 15—Patch Clamping Methods and Analysis of Ion Channels,” Principles of Medical Biology, Vol. 7, Part 2, 355-375 (1997); Xu et al., Drug Discovery Today, 6(24):1278-1287 (2001); and Sullivan et al., Methods Mol. Biol. 114:125-133 (1999). Exemplary experimental methods are also provided in the Examples.

### Diseases and Conditions

Exemplary conditions that can be treated using the compounds described herein include pain (e.g., chronic or acute pain), epilepsy, Alzheimer's disease, Parkinson's disease, diabetes; cancer; sleep disorders; obesity; psychosis such as schizophrenia; overactive bladder; renal disease, neuroprotection, and addiction. For example, the condition can be pain (e.g., neuropathic pain or post-surgery pain), epilepsy, migraine, Parkinson's disease, depression, schizophrenia, psychosis, or tinnitus.

Epilepsy as used herein includes but is not limited to partial seizures such as temporal lobe epilepsy, absence seizures, generalized seizures, and tonic/clonic seizures.

Cancer as used herein includes but is not limited to breast carcinoma, neuroblastoma, retinoblastoma, glioma, prostate carcinoma, esophageal carcinoma, fibrosarcoma, colorectal carcinoma, pheochromocytoma, adrenocarcinoma, insulinoma, lung carcinoma, melanoma, and ovarian cancer.

Acute pain as used herein includes but is not limited to nociceptive pain and post-operative pain. Chronic pain includes but is not limited by: peripheral neuropathic pain such as post-herpetic neuralgia, diabetic neuropathic pain, neuropathic cancer pain, failed back-surgery syndrome, trigeminal neuralgia, and phantom limb pain; central neuropathic pain such as multiple sclerosis related pain, Parkinson disease related pain, post-stroke pain, post-traumatic spinal cord injury pain, and pain in dementia; musculoskeletal pain such as osteoarthritic pain and fibromyalgia syndrome; inflammatory pain such as rheumatoid arthritis and endometriosis; headache such as migraine, cluster headache, tension headache syndrome, facial pain, headache caused by other diseases; visceral pain such as interstitial cystitis, irritable bowel syndrome and chronic pelvic pain syndrome; and mixed pain such as lower back pain, neck and shoulder pain, burning mouth syndrome and complex regional pain syndrome.

In treating osteoarthritic pain, joint mobility can also improve as the underlying chronic pain is reduced. Thus, use of compounds of the present invention to treat osteoarthritic pain inherently includes use of such compounds to improve joint mobility in patients suffering from osteoarthritis.

The compounds described herein can be tested for efficacy in any standard animal model of pain. Various models test the sensitivity of normal animals to intense or noxious stimuli (physiological or nociceptive pain). These tests include responses to thermal, mechanical, or chemical stimuli. Thermal stimuli usually involve the application of hot stimuli (typically varying between 42-55° C.) including, for example: radiant heat to the tail (the tail flick test), radiant heat to the plantar surface of the hindpaw (the Hargreaves test), the hotplate test, and immersion of the hindpaw or tail into hot water. Immersion in cold water, acetone evaporation, or cold plate tests may also be used to test cold pain responsiveness. Tests involving mechanical stimuli typically measure the threshold for eliciting a withdrawal reflex of the hindpaw to graded strength monofilament von Frey hairs or to a sustained pressure stimulus to a paw (e.g., the Ugo Basile analgesiometer). The duration of a response to a standard pinprick may also be measured. When using a chemical stimulus, the response to the application or injection of a chemical irritant (e.g., capsaicin, mustard oil, bradykinin, ATP, formalin, acetic acid) to the skin, muscle joints or internal organs (e.g., bladder or peritoneum) is measured.

In addition, various tests assess pain sensitization by measuring changes in the excitability of the peripheral or central components of the pain neural pathway. In this regard, peripheral sensitization (i.e., changes in the threshold and responsiveness of high threshold nociceptors) can be induced by repeated heat stimuli as well as the application or injection of sensitizing chemicals (e.g., prostaglandins, bradykinin, histamine, serotonin, capsaicin, or mustard oil). Central sensitization (i.e., changes in the excitability of neurons in the central nervous system induced by activity in peripheral pain fibers) can be induced by noxious stimuli (e.g., heat), chemical stimuli (e.g., injection or application of chemical irritants), or electrical activation of sensory fibers.

Various pain tests developed to measure the effect of peripheral inflammation on pain sensitivity can also be used to study the efficacy of the compounds (Stein et al., Pharmacol. Biochem. Behav. (1988) 31: 445-451; Woolf et al., Neurosci. (1994) 62: 327-331). Additionally, various tests assess peripheral neuropathic pain using lesions of the peripheral nervous system. One such example is the “axotomy pain model” (Watson, J. Physiol. (1973) 231:41). Other similar tests include the SNL test which involves the ligation of a spinal segmental nerve (Kim and Chung Pain (1992) 50: 355), the Seltzer model involving partial nerve injury (Seltzer, Pain (1990) 43: 205-18), the spared nerve injury (SNI) model (Decosterd and Woolf, Pain (2000) 87:149), chronic constriction injury (CCl) model (Bennett (1993) Muscle Nerve 16: 1040), tests involving toxic neuropathies such as diabetes (streptozocin model), pyridoxine neuropathy, taxol, vincristine, and other antineoplastic agent-induced neuropathies, tests involving ischaemia to a nerve, peripheral neuritis models (e.g., CFA applied peri-neurally), models of post-herpetic neuralgia using HSV infection, and compression models.

In all of the above tests, outcome measures may be assessed, for example, according to behavior, electrophysiology, neurochemistry, or imaging techniques to detect changes in neural activity.

Exemplary models of pain are also described in the Examples provided herein.

In addition to being able to modulate a particular calcium channel (e.g., Ca_{V} 3.1, Ca_{V} 3.2, or Ca_{V} 3.3), it may be desirable that the compound has very low activity with respect to the hERG K⁺ channel, which is expressed in the heart: compounds that block this channel with high potency may cause reactions which are fatal. See, e.g., Bowlby et al., “hERG (KCNH2 or K_{V}11.1 K⁺ Channels: Screening for Cardiac Arrhythmia Risk,” Curr. Drug Metab. 9(9):965-70 (2008)). Thus, for a compound that modulates calcium channel activity, it may also be shown that the hERG K⁺ channel is not inhibited or only minimally inhibited as compared to the inhibition of the primary channel targeted. Similarly, it may be desirable that the compound does not inhibit cytochrome p450, an enzyme that is required for drug detoxification. Such compounds may be particularly useful in the methods described herein.

It is known that calcium channel activity is involved in a multiplicity of disorders, and particular types of channels are associated with particular conditions. The association of T-type channels in conditions associated with neural transmission would indicate that compounds of the invention which target T-type receptors are most useful in these conditions. The compounds described herein, e.g., a compound according to any of Formulas (I)-(XIII) or any of Compounds 1-75 in Table 1, can exhibit a high selectivity for T-type channels. Thus, as described below, they can be studied for their ability to interact specifically with T-type channels as an indication of desirable function and selectiviey. It is desirable that the compounds exhibit IC₅₀ values of <1 μM for T-type calcium channels. In one embodiment, the IC₅₀ is less than 0.50 μM. In one embodiment the IC₅₀ is less than 0.01 μM. In one embodiment, the IC₅₀ is between 0.01 μM and 0.1 μM. In still another embodiment, the IC₅₀ is between 0.1 μM and 0.5 μM. In other embodiments, the IC₅₀ is between 0.5-1 μM. The IC₅₀ is the concentration which inhibits 50% of the calcium, barium or other permeant divalent cation flux at a particular applied potential.

Compound 3 from Table 1 herein was tested in such additional assays, and exhibited negligible hERG activity, and less than 10% inhibition of various cytochromes (2C9, 2D6, 3A4) at 10 micromolar.

The compounds of the invention modulate the activity of calcium channels; in general, said modulation is the inhibition of the ability of the channel to transport calcium. As described below, the effect of a particular compound on calcium channel activity can readily be ascertained in a routine assay whereby the conditions are arranged so that the channel is activated, and the effect of the compound on this activation (either positive or negative) is assessed. Exemplary assays are also described in the Examples.

### Pharmaceutical Compositions

For use as treatment of human and animal subjects, the compounds of the invention can be formulated as pharmaceutical or veterinary compositions. Depending on the subject to be treated, the mode of administration, and the type of treatment desired—e.g., prevention, prophylaxis, or therapy—the compounds are formulated in ways consonant with these parameters. A summary of such techniques is found in Remington: The Science and Practice of Pharmacy, 21^{st} Edition, Lippincott Williams & Wilkins, (2005); and Encyclopedia of Pharmaceutical Technology, eds. J. Swarbrick and J. C. Boylan, 1988-1999, Marcel Dekker, New York, each of which is incorporated herein by reference.

The compounds described herein (e.g., a compound according to any of Formulas (I)-(XIII) or any of Compounds 1-75 in Table 1) may be present in amounts totaling 1-95% by weight of the total weight of the composition. The composition may be provided in a dosage form that is suitable for intraarticular, oral, parenteral (e.g., intravenous, intramuscular), rectal, cutaneous, subcutaneous, topical, transdermal, sublingual, nasal, vaginal, intravesicular, intraurethral, intrathecal, epidural, aural, or ocular administration, or by injection, inhalation, or direct contact with the nasal, genitourinary, gastrointesitnal, reproductive or oral mucosa. Thus, the pharmaceutical composition may be in the form of, e.g., tablets, capsules, pills, powders, granulates, suspensions, emulsions, solutions, gels including hydrogels, pastes, ointments, creams, plasters, drenches, osmotic delivery devices, suppositories, enemas, injectables, implants, sprays, preparations suitable for iontophoretic delivery, or aerosols. The compositions may be formulated according to conventional pharmaceutical practice.

In general, for use in treatment, the compounds described herein (e.g., a compound according to any of Formulas (I)-(XIII) or any of Compounds 1-75 in Table 1) may be used alone, as mixtures of two or more compounds or in combination with other pharmaceuticals. An example of other pharmaceuticals to combine with the compounds described herein (e.g., a compound according to any of Formulas (I)-(XIII) or any of Compounds 1-75 in Table 1) would include pharmaceuticals for the treatment of the same indication. For example, in the treatment of pain, a compound may be combined with another pain relief treatment such as an NSAID, or a compound which selectively inhibits COX-2, or an opioid, or an adjuvant analgesic such as an antidepressant. Another example of a potential pharmaceutical to combine with the compounds described herein (e.g., a compound according to any of Formulas (I)-(XIII) or any of Compounds 1-75 in Table 1) would include pharmaceuticals for the treatment of different yet associated or related symptoms or indications. Depending on the mode of administration, the compounds will be formulated into suitable compositions to permit facile delivery. Each compound of a combination therapy may be formulated in a variety of ways that are known in the art. For example, the first and second agents of the combination therapy may be formulated together or separately. Desirably, the first and second agents are formulated together for the simultaneous or near simultaneous administration of the agents.

The compounds of the invention may be prepared and used as pharmaceutical compositions comprising an effective amount of a compound described herein (e.g., a compound according to any of Formulas (I)-(XIII) or any of Compounds 1-75 in Table 1) and a pharmaceutically acceptable carrier or excipient, as is well known in the art. In some embodiments, the composition includes at least two different pharmaceutically acceptable excipients or carriers.

Formulations may be prepared in a manner suitable for systemic administration or topical or local administration. Systemic formulations include those designed for injection (e.g., intramuscular, intravenous or subcutaneous injection) or may be prepared for transdermal, transmucosal, or oral administration. The formulation will generally include a diluent as well as, in some cases, adjuvants, buffers, preservatives and the like. The compounds can be administered also in liposomal compositions or as microemulsions.

For injection, formulations can be prepared in conventional forms as liquid solutions or suspensions or as solid forms suitable for solution or suspension in liquid prior to injection or as emulsions. Suitable excipients include, for example, water, saline, dextrose, glycerol and the like. Such compositions may also contain amounts of nontoxic auxiliary substances such as wetting or emulsifying agents, pH buffering agents and the like, such as, for example, sodium acetate, sorbitan monolaurate, and so forth.

Various sustained release systems for drugs have also been devised. See, for example, U.S. Pat. No. 5,624,677, which is herein incorporated by reference.

Systemic administration may also include relatively noninvasive methods such as the use of suppositories, transdermal patches, transmucosal delivery and intranasal administration. Oral administration is also suitable for compounds of the invention. Suitable forms include syrups, capsules, and tablets, as is understood in the art.

For administration to animal or human subjects, the dosage of the compounds of the invention may be, for example, 0.01-50 mg/kg (e.g., 0.01-15 mg/kg or 0.1-10 mg/kg). For example, the dosage can be 10-30 mg/kg.

Each compound of a combination therapy, as described herein, may be formulated in a variety of ways that are known in the art. For example, the first and second agents of the combination therapy may be formulated together or separately.

The individually or separately formulated agents can be packaged together as a kit. Non-limiting examples include, but are not limited to, kits that contain, e.g., two pills, a pill and a powder, a suppository and a liquid in a vial, two topical creams, etc. The kit can include optional components that aid in the administration of the unit dose to patients, such as vials for reconstituting powder forms, syringes for injection, customized IV delivery systems, inhalers, etc. Additionally, the unit dose kit can contain instructions for preparation and administration of the compositions. The kit may be manufactured as a single use unit dose for one patient, multiple uses for a particular patient (at a constant dose or in which the individual compounds may vary in potency as therapy progresses); or the kit may contain multiple doses suitable for administration to multiple patients (“bulk packaging”). The kit components may be assembled in cartons, blister packs, bottles, tubes, and the like.

Formulations for oral use include tablets containing the active ingredient(s) in a mixture with non-toxic pharmaceutically acceptable excipients. These excipients may be, for example, inert diluents or fillers (e.g., sucrose, sorbitol, sugar, mannitol, microcrystalline cellulose, starches including potato starch, calcium carbonate, sodium chloride, lactose, calcium phosphate, calcium sulfate, or sodium phosphate); granulating and disintegrating agents (e.g., cellulose derivatives including microcrystalline cellulose, starches including potato starch, croscarmellose sodium, alginates, or alginic acid); binding agents (e.g., sucrose, glucose, sorbitol, acacia, alginic acid, sodium alginate, gelatin, starch, pregelatinized starch, microcrystalline cellulose, magnesium aluminum silicate, carboxymethylcellulose sodium, methylcellulose, hydroxypropyl methylcellulose, ethylcellulose, polyvinylpyrrolidone, or polyethylene glycol); and lubricating agents, glidants, and antiadhesives (e.g., magnesium stearate, zinc stearate, stearic acid, silicas, hydrogenated vegetable oils, or talc). Other pharmaceutically acceptable excipients can be colorants, flavoring agents, plasticizers, humectants, buffering agents, and the like.

Two or more compounds may be mixed together in a tablet, capsule, or other vehicle, or may be partitioned. In one example, the first compound is contained on the inside of the tablet, and the second compound is on the outside, such that a substantial portion of the second compound is released prior to the release of the first compound.

Formulations for oral use may also be provided as chewable tablets, or as hard gelatin capsules wherein the active ingredient is mixed with an inert solid diluent (e.g., potato starch, lactose, microcrystalline cellulose, calcium carbonate, calcium phosphate or kaolin), or as soft gelatin capsules wherein the active ingredient is mixed with water or an oil medium, for example, peanut oil, liquid paraffin, or olive oil. Powders, granulates, and pellets may be prepared using the ingredients mentioned above under tablets and capsules in a conventional manner using, e.g., a mixer, a fluid bed apparatus or a spray drying equipment.

Dissolution or diffusion controlled release can be achieved by appropriate coating of a tablet, capsule, pellet, or granulate formulation of compounds, or by incorporating the compound into an appropriate matrix. A controlled release coating may include one or more of the coating substances mentioned above and/or, e.g., shellac, beeswax, glycowax, castor wax, carnauba wax, stearyl alcohol, glyceryl monostearate, glyceryl distearate, glycerol palmitostearate, ethylcellulose, acrylic resins, dl-polylactic acid, cellulose acetate butyrate, polyvinyl chloride, polyvinyl acetate, vinyl pyrrolidone, polyethylene, polymethacrylate, methylmethacrylate, 2-hydroxymethacrylate, methacrylate hydrogels, 1,3 butylene glycol, ethylene glycol methacrylate, and/or polyethylene glycols. In a controlled release matrix formulation, the matrix material may also include, e.g., hydrated methylcellulose, carnauba wax and stearyl alcohol, carbopol 934, silicone, glyceryl tristearate, methyl acrylate-methyl methacrylate, polyvinyl chloride, polyethylene, and/or halogenated fluorocarbon.

The liquid forms in which the compounds and compositions of the present invention can be incorporated for administration orally include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles.

Generally, when administered to a human, the oral dosage of any of the compounds of the combination of the invention will depend on the nature of the compound, and can readily be determined by one skilled in the art. Typically, such dosage is normally about 0.001 mg to 2000 mg per day, desirably about 1 mg to 1000 mg per day, and more desirably about 5 mg to 500 mg per day. Dosages up to 200 mg per day may be necessary.

Administration of each drug in a combination therapy, as described herein, can, independently, be one to four times daily for one day to one year, and may even be for the life of the patient. Chronic, long-term administration may be indicated.

### Synthesis

The following reaction schemes and examples are intended to illustrate the synthesis of a representative number of compounds. Accordingly, the following examples are intended to illustrate but not to limit the invention. Additional compounds not specifically exemplified may be synthesized using conventional methods and known starting materials in combination with the methods described hereinbelow.

### Example 1

### Synthesis of N-(3,5-bis(trifluoromethyl)phenyl)-2-(piperidin-4-yl)acetamide hydrochloride (4)

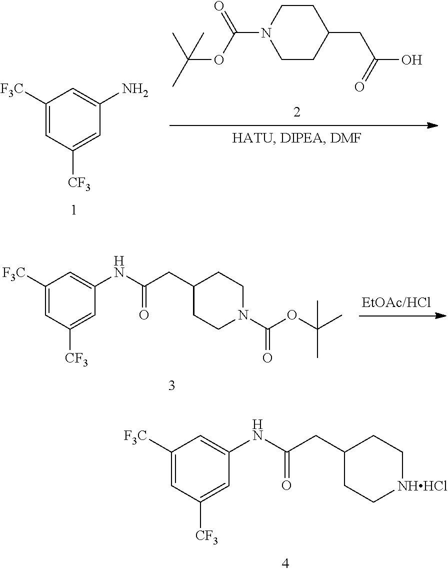

3,5-Bis(trifluoromethyl)aniline (1) (20.6 g, 90.0 mmol), 2-(1-(tert-butoxycarbonyl)piperidin-4-yl)acetic acid (2) (19.5 g, 80.2 mmol), DIPEA (12.9 g, 100 mmol) and HATU (31.5 g, 82.9 mmol) were stirred in DMF (150 mL) at 50° C. for 48 h. The solvent was removed in vacuo, and the residue taken up in DCM, washed sequentially with saturated NH₄Cl solution, saturated NaHCO₃ solution, and water, dried, and concentrated in-vacuo. The residue was purified by flash column chromatography (15-30% EtOAc/PE) followed by recrystallization from EtOAc/hexanes to give tert-butyl 4-(2-(3,5-bis(trifluoromethyl)phenylamino)-2-oxoethyl)piperidine-1-carboxylate (3).

The solid was dissolved in EtOAc (150 mL), HCl (g) was bubbled through the solution for 5 min, and the reaction was stirred at room temperature for 30 min and at 0° C. for 30 min. The resultant solid was collected by filtration to give N-(3,5-bis(trifluoromethyl)phenyl)-2-(piperidin-4-yl)acetamide hydrochloride (4) (24.0 g, 77%): ¹H NMR (300 mHz—D₂O) δ 1.41 (q, 2H, J=11.4 Hz), 1.88 (d, J=2 H, J=14.3 Hz), 2.03 (m, 1H), 2.35 (d, 2H, J=7.14 Hz), 2.91 (t, 2H, 12.78 Hz), 3.33 (d, 2 H, J=12.6 Hz), 7.74 (s, 1H), 7.87 (s, 2H).

### Example 2

### Synthesis of N-(3,5-bis(trifluoromethyl)phenyl)-2-(pyrrolidin-3-yl)acetamide (7)

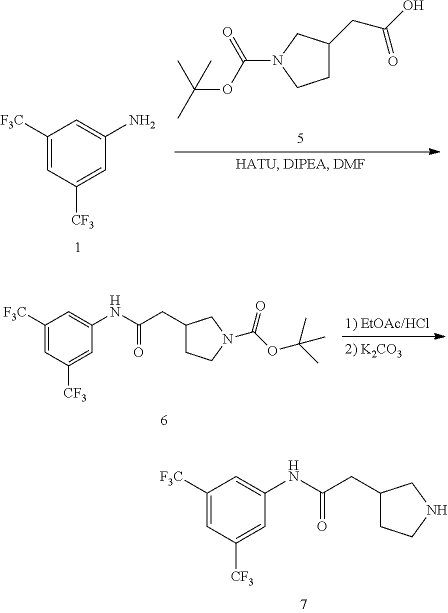

3,5-Bis(trifluoromethyl)aniline (1) (4.58 g, 20.0 mmol), N-Boc-3-pyrrolidine acetic acid (5) (4.0 g, 17.4 mmol), DIPEA (5.2 mL, 30 mmol) and HATU (9.5 g, 25 mmol) were stirred in DMF (40 mL) at 40° C. for 72 h. The reaction was diluted with saturated NH₄Cl solution, extracted with Et₂O, washed with saturated NaHCO₃ solution, dried, and concentrated in vacuo. The residue was purified by automated column chromatography (0-40% EtOAc, PE) to give tert-butyl 3-(2-(3,5-bis(trifluoromethyl)phenylamino)-2-oxoethyl)pyrrolidine-1-carboxylate (6).

The solid was taken up in EtOAc (30 mL), HCl (g) was bubbled through the solution for 30 secs, then it was stirred at room temperature for 1 h and concentrated in-vacuo. The residue was taken up in EtOAc/PE (1/5). The resultant precipitate was collected by filtration, dissolved in H₂O (50 mL), basified with saturated K₂CO₃ solution, and extracted with EtOAc. The organics were dried and concentrated in vacuo to give N-(3,5-bis(trifluoromethyl)phenyl)-2-(pyrrolidin-3-yl)acetamide (7) (4.8 g, 81%). The product structure was confirmed by LCMS.

### Example 3

### Synthesis of 2-(azetidin-3-yl)-N-(3,5-bis(trifluoromethyl)phenyl)acetamide (10)

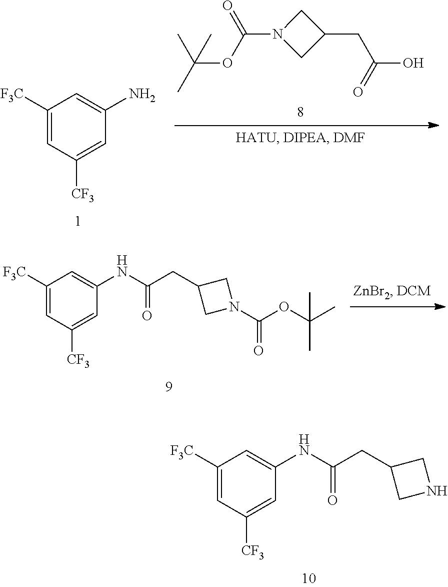

3,5-Bis(trifluoromethyl)aniline (1) (5.04 g, 22.0 mmol), 2-(1-(tert-butoxycarbonyl)azetidin-3-yl)acetic acid (8) (4.5 g, 20.9 mmol), DIPEA (5.2 mL, 30 mmol) and HATU (10.6 g, 28 mmol) were stirred in DMF (40 mL) at 40° C. for 72 h. The reaction was diluted with saturated NH₄Cl solution, extracted with Et₂O, washed with saturated NaHCO₃ solution, dried, concentrated in-vacuo and the residue purified by automated column chromatography (0-50% EtOAc, PE) to provide tert-butyl 3-(2-(3,5-bis(trifluoromethyl)phenylamino)-2-oxoethyl)azetidine-1-carboxylate (9). A mixture of this compound (2.75 g, 6.46 mmol) and ZnBr₂ (5.00 g, 22.2 mmol), was stirred in the presence of molecular sieves in DCM at room temperature for 6 h. NH₃OH/H₂O (30 mL/30 mL) was added, and the reaction stirred at room temperature for 2 h. At this time, the organics were separated (extracting with additional DCM), and the combined organics were dried and concentrated in vacuo to give 2-(azetidin-3-yl)-N-(3,5-bis(trifluoromethyl)phenyl)acetamide (10) (1.91 g, 90%), estimated 75% purity by ¹H NMR; ¹H NMR (300 mHz—CDCl₃) δ 2.71 (m, 6H), 3.06 (m, 2H), 3.34 (m, 2H), 3.60 (m, 1H), 3.93 (m, 3H), 7.50 (s, 1H), 8.0 (s, 2H), 9.58 (bs, 1H). The product was used without further purification.

### Example 4

### Synthesis of 1-(3,5-bis(trifluoromethyl)phenyl)-3-(piperidin-4-ylmethyl)urea (14)

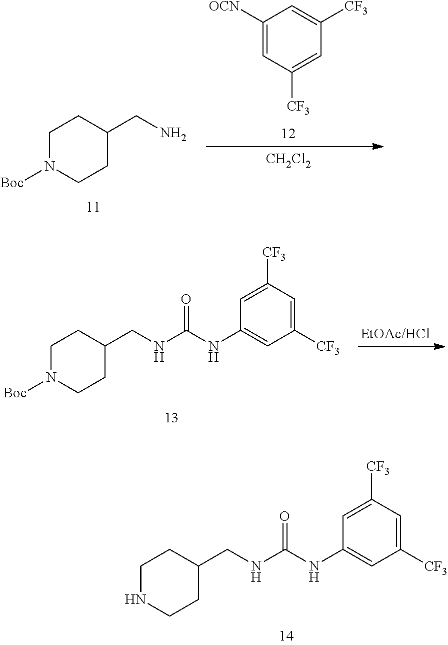

To a solution of tert-butyl 4-(aminomethyl)piperidine-1-carboxylate (11) (2.05 g, 9.57 mmol) in CH₂Cl₂ (110 mL) at 0° C. was added slowly 1-isocyanato-3,5-bis(trifluoromethyl)benzene (12)(1.65 mL, 9.57 mmol). The reaction was stirred for 4 hours; at this time, the reaction was concentrated and purified by automated flash chromatography (R_{f}=0.6 in 1:1 PE:EtOAc) to provide the title compound as a white solid (3.93 g, 88%). ¹H NMR (300 mHz, CDCl₃) δ 1.12 (m, 2H), 1.47 (s, 9H), 1.72 (m, 4H), 2.74 (t, 2H, J=12.8 Hz), 3.20 (m, 2H), 4.13 (d, 2H, J=11.7 Hz), 5.74 (br s, 1H), 7.45 (s, 1H), 7.88 (s, 2H). LRMS (ESI) calcd for C₁₅H₁₇F₆N₃O [M-Boc+2] 370.3, found 370.0, calcd for C₂₀H₂₅F₆N₃O₃ [M+Na] 492.4, found 492.0].

A solution of tert-butyl 4-((3-(3,5-bis(trifluoromethyl)phenyl)ureido)methyl)piperidine-1-carboxylate (13) (1.15 g, 2.45 mmol) in EtOAc (40 mL) was bubbled with HCl gas for 45 seconds. The solution was then concentrated after stirring at room temperature for 45 minutes to provide the product in quantitative yield as an HCl salt. LRMS (ESI) calcd for C₁₅H₁₇F₆N₃O [M+1] 370.3, found 370.0.

### Example 5

### Synthesis of 1-(3,5-bis(trifluoromethyl)phenyl)-3-(piperidin-4-yl)urea (17)

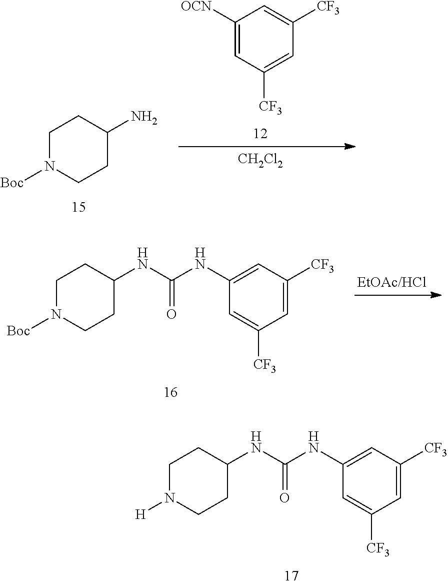

To a solution of tert-butyl 4-aminopiperidine-1-carboxylate (15) (1.11 g, 5.54 mmol) in CH₂Cl₂ (100 mL) was added slowly 1-isocyanato-3,5-bis(trifluoromethyl)benzene (12) (0.96 mL, 5.54 mmol). The reaction was stirred at room temperature overnight. The reaction was then concentrated, and the residue was purified by automated flash chromatography (R_{f}=0.65 in 1:1 PE:EtOAc) to provide the title compound in quantitative yield as a white foam. ¹H NMR (300 mHz, CDCl₃) δ 1.24 (m, 2H), 1.48 (s, 9H), 1.98 (d, 2H, J=11.9 Hz), 2.91 (d, 2H, J=11.4 Hz), 3.86 (br s, 1H), 4.02 (d, 2H, J=13.6 Hz), 5.37 (br s, 1H), 7.47 (s, 1H), 7.86 (s, 2 H). LRMS (ESI) calcd for C₁₄H₁₅F₆N₃O [M-BOC+2] 356.3, found 356.0, calcd for C₂₉H₂₃F₆N₃O₃ [M+Na] 478.4, found 478.0].

A solution of tert-butyl 4-(3-(3,5-bis(trifluoromethyl)phenyl)ureido)piperidine-1-carboxylate (16) (1.29 g, 2.83 mmol) in EtOAc (40 mL) was saturated with HCl gas. The reaction was stirred at room temperature for 45 minutes, then concentrated to provide the product as an HCl salt in quantitative yield. LRMS (ESI) calcd for C₁₄H₁₅F₆N₃O [M+1] 356.3, found 356.0.

### Example 6

### Synthesis of 3-(tert-butoxycarbonylamino)-2,2-dimethylpropanoic acid (19)

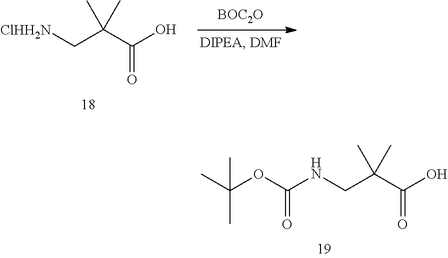

3-Amino-2,2-dimethylpropanoic acid (18) (1.2 g, 10.2 mmol), Boc anhydride (2.3 g, 10.2 mmol) and DIPEA (1.85 mL, 10.23 mmol) were stirred under argon in DMF (30 mL) at 60° C. for 16 h. The reaction was concentrated in vacuo, taken up in EtOAc, washed with saturated NH₄Cl solution, and concentrated in vacuo to give 3-(tert-butoxycarbonylamino)-2,2-dimethylpropanoic acid (19) (2.0 g, 91%); ¹H NMR (300 mHz—CDCl₃) δ 1.33 (s, 6H), 1.37 (s, 9H), 2.69 (s, 2H).

### Example 7

### Synthesis of 3-(tert-butoxycarbonylamino)-3-methylbutanoic acid (20)

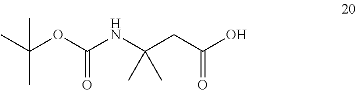

3-(tert-butoxycarbonylamino)-3-methylbutanoic acid (20) was prepared in analogous manner to Example 5, using 3-amino-3-methylbutanoic acid hydrochloride (19).

### Example 8

### General Protocol for BOC Amino Acids Amide Coupling Exemplified by the Synthesis of 2-(1-((trans)-2-aminocyclohexanecarbonyl)piperidin-4-yl)-N-(3,5-bis(trifluoromethyl)phenyl)acetamide (23)

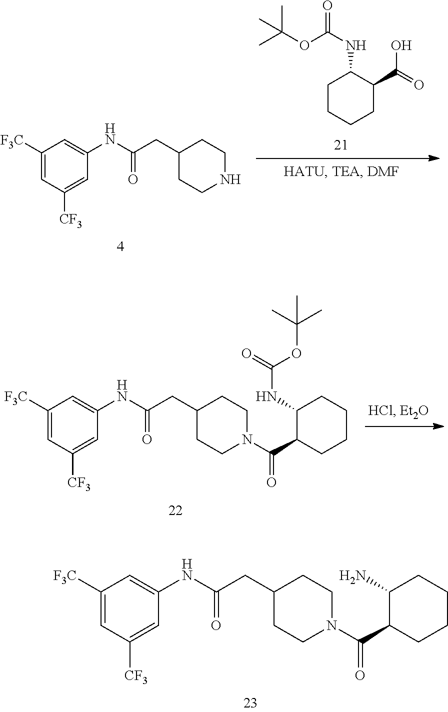

N-(3,5-bis(trifluoromethyl)phenyl)-2-(piperidin-4-yl)acetamide (free base of Compound 4; 100 mg, 0.28 mmol), HATU (161 mg, 0.42 mmol), TEA (197 μL, 1.41 mmol) and (trans)-2-(tert-butoxycarbonylamino)cyclohexanecarboxylic acid (21) (103 mg, 0.42 mmol) were stirred in DMF (1 mL) at room temperature for 16 h. The reaction was scavenged with Si bound isocyanate and Si bound carbonate resins, and filtered. The filtrate was then concentrated in-vacuo. The residue was treated with 2M HCl in Et₂O at room temperature for 5 h, and quenched with saturated NaHCO₃ solution. The organics were separated, dried, and concentrated in vacuo. The residue was purified by mass directed reverse phase HPLC to give 2-(1-((trans)-2-aminocyclohexane carbonyl)-piperidin-4-yl)-N-(3,5-bis(trifluoromethyl)phenyl)acetamide (23).

### Example 9

### Synthesis of N-(3,5-bis(trifluoromethyl)phenyl)-2-(1-(1-methylpiperidine-3-carbonyl)piperidin-4-yl)acetamide (25)

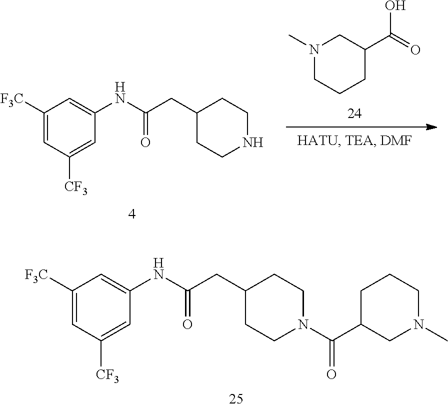

N-(3,5-bis(trifluoromethyl)phenyl)-2-(piperidin-4-yl)acetamide (free base of Compound 4; 100 mg, 0.28 mmol), HATU (161 mg, 0.42 mmol), TEA (197 μL, 1.41 mmol) and 1-methylpiperidine-3-carboxylic acid (24) (60 mg, 0.42 mmol) were stirred in DMF (1 mL) at room temperature for 16 h. The reaction was scavenged with Si bound isocyanate and Si bound carbonate resin and filtered. The filtrate was then concentrated in-vacuo. The residue was purified by mass directed reverse phase HPLC to give N-(3,5-bis(trifluoromethyl)phenyl)-2-(1-(1-methylpiperidine-3-carbonyl)piperidin-4-yl)acetamide (25).

### Example 10

### Synthesis of 2-(1-(trans)-(2-aminocyclohexanecarbonyl)piperidin-4-yl)-N-(3,5-dichlorophenyl)acetamide (30)

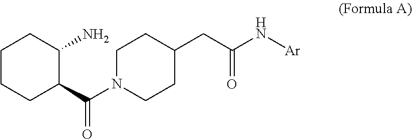

A general method for the synthesis of analogs of Formula A is exemplified by the synthesis of 2-(1-(trans)-(2-aminocyclohexanecarbonyl)piperidin-4-yl)-N-(3,5-dichlorophenyl)acetamide (30). The synthesis of compound (27) is described in Example 11.

### Example 11

### Synthesis of ethyl 2-(1-trans-(2-(tert-butoxycarbonylamino)cyclohexanecarbonyl)-piperidin-4-yl)acetate (27)

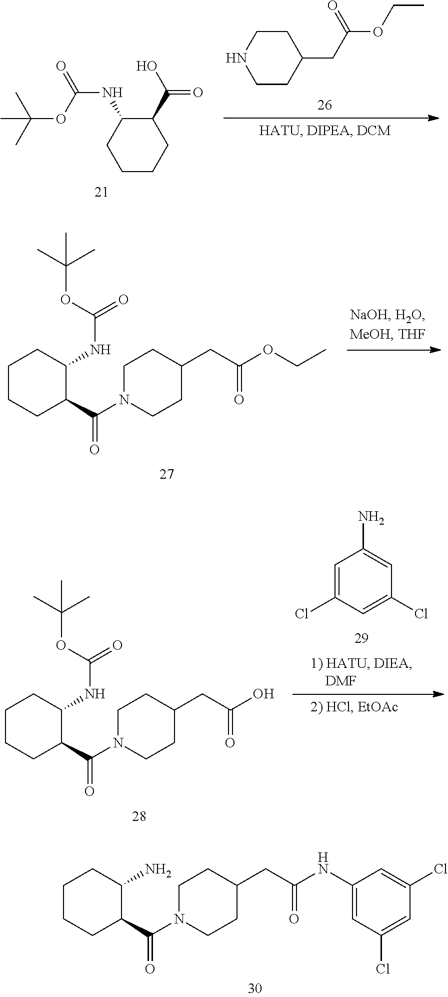

Trans-2-(tert-butoxycarbonylamino)cyclohexanecarboxylic acid (21) (1.5 g, 6.17 mmol), ethyl 2-(piperidin-4-yl)acetate (26) (1.06 g, 6.12 mmol), HATU (3.05 g, 8.02 mmol), and DIPEA (5.37 mL, 30.85 mmol) were stirred in DCM (120 mL) at room temperature for 16 h. The reaction was concentrated in vacuo and taken up in EtOAc. The organic layer was washed sequentially with saturated NaHCO₃ solution, saturated NH₄Cl solution, saturated NaHCO₃ solution, and brine. The organic layer was then dried and concentrated in vacuo. The residue was then purified by automated column chromatography (50% EtOAc/PE) to give ethyl 2-(1-trans-(2-(tert-butoxycarbonylamino)cyclohexanecarbonyl)-piperidin-4-yl)acetate (27) (2.82 g, 100%). ¹H NMR (300 mHz—CD₃OD) δ 1.33 (m, 18H), 2.02 (m, 7H), 2.28 (m, 2 H), 2.59 (m, 1H), 2.80 (m, 1H), 3.09 (m, 1H), 3.65 (m, 1H), 4.13 (m, 3H), 4.54 (m, 1H), 6.45 (m, 1H).

### Preparation of 2-(1-((trans)-2-(tert-butoxycarbonylamino)cyclohexanecarbonyl)piperidin-4-yl)acetic acid (28)

Ethyl 2-(1-trans-(2-(tert-butoxycarbonylamino)cyclohexanecarbonyl)-piperidin-4-yl)acetate (27) (2.82 g, 7.11 mmol) and NaOH (1.02 g, 17.3 mmol) were heated in MeOH/THF/H₂O (20/80/20 mL) at reflux for 16 h. The reaction was concentrated in vacuo, and the residue was then taken up in H₂O (20 mL) and acidified with 2 M HCl. The aqueous was extracted with three times with EtOAc, and the combined organics dried and concentrated in-vacuo to give 2-(1-((trans)-2-(tert-butoxycarbonylamino)cyclohexanecarbonyl)piperidin-4-yl)acetic acid (28) (2.22 g, 85%). ¹H NMR (300 mHz—CD₃OD) δ 1.30 (m, 16H), 1.81 (m, 6H), 2.26 (d, 2H, J=7.08 Hz), 2.63 (m, 1H), 2.81 (m, 1H), 3.11 (m, 1H), 3.64 (m, 1H), 4.55 (m, 1H).

### Preparation of 2-(1-(trans)-(2-aminocyclohexanecarbonyl)piperidin-4-yl)-N-(3,5-dichlorophenyl)acetamide (30)

2-(1-((Trans)-2-(tert-butoxycarbonylamino)cyclohexanecarbonyl)piperidin-4-yl)acetic acid (28) (100 mg, 0.27 mmol), 3,5-dichloroaniline (29) (57 mg, 0.35 mmol), HATU (134 mg, 0.35 mmol) and DIPEA (236 μL, 1.36 mmol) were stirred in DMF (0.5 mL) at room temperature for 16 h. The reaction was diluted with EtOAc (2.5 mL), washed with saturated NaHCO₃ solution, and the organic layer ws then treated with HCl (g) for 12 s. The reaction was concentrated in-vacuo and the residue purified by mass directed reverse phase HPLC to give 2-(1-(trans)-(2-aminocyclohexanecarbonyl)piperidin-4-yl)-N-(3,5-dichlorophenyl)acetamide (30).

### Example 12

### General Procedure for the Synthesis of N-(3,5-bis(trifluoromethyl)phenyl)-2-(4-hydroxypiperidin-4-yl)acetamide hydrochloride (33)

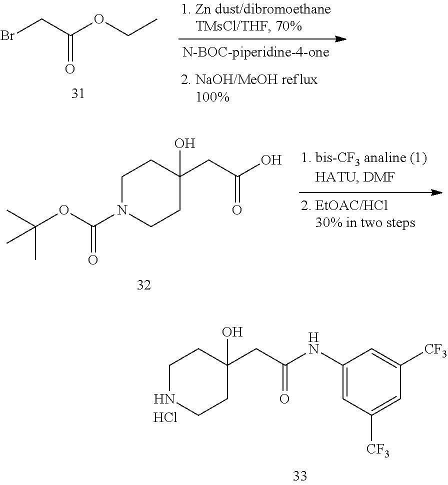

To a round-bottom flask containing Zn dust (878 mg, 13.47 mmol) was added dibromoethane (0.1 mL, 1.16 mmol). The resulting mixture was warmed to 60° C. and allowed to cool for 1 min. This heating-cooling process was repeated three more times, and then the flask was allowed to cool for an additional 3 min. Trimethylsilyl chloride (0.2 mL, 1.56 mmol) in THF (15 mL) was added, followed by addition of ethyl-2-bromoacetate (31) (0.5 ml, 4.49 mmol) in THF (3 mL). The reaction was warmed to 60° C. for an additional two hours until a dark grey suspension was obtained. The mixture was cooled to room temperature; N—BOC-piperidine-4-one (600 mg, 3.0 mmol) in THF (20 mL) was then added. The resulting mixture was continued to stir for 3 days then quenched with water. The solid was filtered off, and the aqueous was extracted with ethyl acetate. The combined organic layers were washed with brine and dried over Na₂SO₄. Purification was performed in Biotage to give the product as colorless oil (730 mg, 85%).

The material (730 mg) from the previous step was dissolved in the mixture of methanol (5 mL) and NaOH solution (10 N, 1 mL). The resulting mixture was refluxed for 3 hours and then cooled to room temperature. The solvent was evaporated, and the residue was re-dissolved in water and then extracted with diethyl ether. The aqueous solution was neutralized with conc. HCl until pH=4, and the aqueous layer was then extracted with dichloromethane. The combined dichloromethane layers were dried over NaSO₄. Evaporation of solvent gave 32 as white solid. ¹H NMR (300 mHz; CDCl₃) δ 1.44 (s, 9H), 1.48 (m, 2H), 1.66 (d, 2H, J=12.99 Hz), 2.46 (s, 2H), 3.13 (t, 2H, J=11.7 Hz), 3.76 (b, 2H).

### Example 13

### Synthesis of N-(3,5-bis(trifluoromethyl)phenyl)-2-(4-hydroxypiperidin-4-yl)acetamide hydrochloride (33)

To a solution of 32 (665 mg, 2.56 mmol) in DMF (10 mL) were added 3, 5-bis-CF₃-aniline (1.6 g, 7.08 mmol), di-isopropylethyl amine (1.3 mL, 7.10 mmol), and HATU (2.7 g, 7.10 mmol). The resulting mixture was stirred for 3 days at 60° C. The solvent was evaporated, and the residue was dissolved in EtOAc and washed sequentially with water and brine. The organic fraction was dried over Na₂SO₄, filtered, and the solvent was removed under reduced pressure. The crude material was purified by Biotage to provide the product as a white solid (540 mg, 32%).

The material obtained from above was dissolved in EtOAc, and HCl gas was bubbled into solution for 30 seconds. The resulting solution was capped and stirred for an additional 1 hour at room temperature. Evaporation of solvent gave the compound 33 as HCl salt. ¹H NMR (300 mHz; CD₃OD) δ 2.01 (b, 4H), 2.67 (s, 2 H), 3.42 (m, 4H), 7.67 (s, 1H), 8.26 (s, 2H).

### Example 14

### Mass Spectrometric Analysis

Following the general procedures set forth in Examples 1-13, the following compounds listed in Table 1 below were prepared. Mass spectrometry was employed with the final compound and at various stages throughout the synthesis as a confirmation of the identity of the product obtained (M+1). For the mass spectrometric analysis, samples were prepared at an approximate concentration of 1 μg/mL in methanol:water (50:50 v/v) with 0.1% formic acid. Samples were then analyzed by a Waters 3100 Applied Biosystems API3000 single quadrupole mass spectrometer and scanned in the range of 250 to 700 m/z.

The compound numbers used in this table do not correspond to the numbering of compounds in the synthesis schemes and examples, and all references to testing of compounds refers to the compound numbers in Table 1.

Note also that, for convenience, a single enantiomer is depicted for many of the chiral compounds, to clearly illustrate that a certain diastereomer is intended. However, the compounds in Table 1 are all racemic unless the Compound Name in the Table indicates a specific chirality. Compounds for which the name designates a specific enantiomer, by use of chirality designations R and S, were obtained and evaluated in optically active form, and are generally at least about 90% optically pure.

TABLE 1
Compounds of Formula (I)

| Cmpd. | | | |
|---|---|---|---|
| No. | Structure | Cmpd Name | MW |
| 1 | 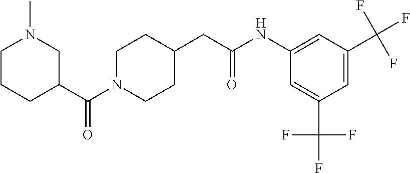 | N-(3,5- bis(trifluoromethyl) phenyl)-2-(1-(1- methylpiperidine-3- carbonyl)piperidin-4- yl)acetamide | 479.46 |
| 2 | 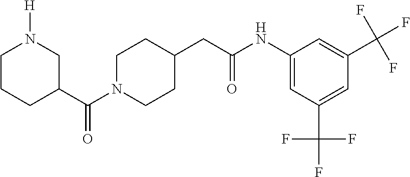 | N-(3,5- bis(trifluoromethyl) phenyl)-2-(1- (piperidine-3- carbonyl)piperidin-4- yl)acetamide | 465.43 |
| 3 | 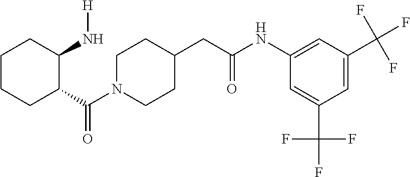 | trans-2-(1-(2- aminocyclohexanecarbonyl) piperidin-4- yl)-N-(3,5- bis(trifluoromethyl) phenyl)acetamide | 479.46 |
| 4 | 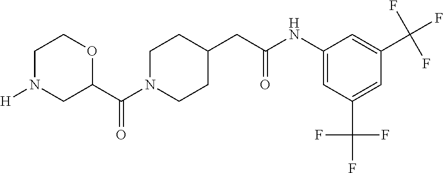 | N-(3,5- bis(trifluoromethyl) phenyl)-2-(1- (morpholine-2- carbonyl)piperidin-4- yl)acetamide | 467.41 |
| 5 | 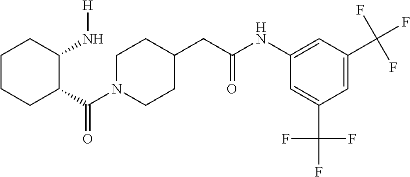 | cis-2-(1-(2- aminocyclohexanecarbonyl) piperidin-4- yl)-N-(3,5- bis(trifluoromethyl) phenyl)acetamide | 479.46 |
| 6 | 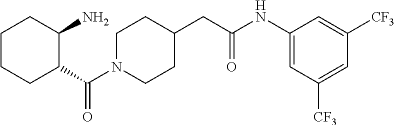 | 2-(1-((1R,2R)-2- aminocyclohexanecarbonyl) piperidin-4- yl)-N-(3,5- bis(trifluoromethyl) phenyl)acetamide | 479.46 |
| 7 | 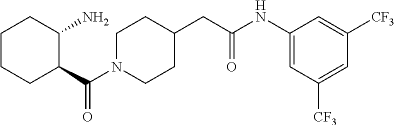 | 2-(1-((1S,2S)-2- aminocyclohexanecarbonyl) piperidin-4- yl)-N-(3,5- bis(trifluoromethyl) phenyl)acetamide | 479.46 |
| 8 | 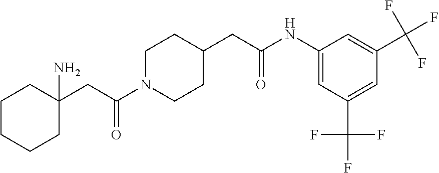 | 2-(1-(2-(1- aminocyclohexyl) acetyl)piperidin-4-yl)-N- (3,5- bis(trifluoromethyl) phenyl)acetamide | 493.49 |
| 9 | 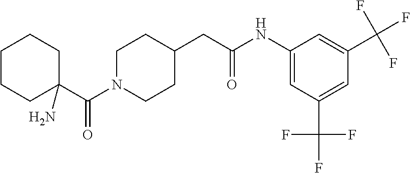 | 2-(1-(1- aminocyclohexanecarbonyl) piperidin-4- yl)-N-(3,5- bis(trifluoromethyl) phenyl)acetamide | 479.46 |
| 10 | 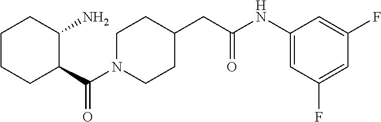 | trans-2-(1-(2- aminocyclohexanecarbonyl) piperidin-4- yl)-N-(3,5- difluorophenyl)acetamide | 379.44 |
| 11 | 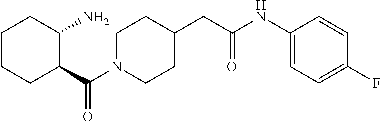 | trans-2-(1-(2- aminocyclohexanecarbonyl) piperidin-4- yl)-N-(4- fluorophenyl)acetamide | 361.45 |
| 12 | 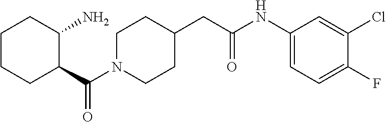 | trans-2-(1-(2- aminocyclohexanecarbonyl) piperidin-4- yl)-N-(3-chloro-4- fluorophenyl)acetamide | 395.90 |
| 13 | 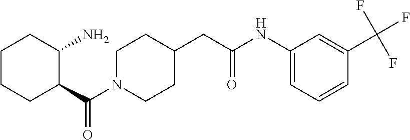 | trans-2-(1-(2- aminocyclohexanecarbonyl) piperidin-4- yl)-N-(3- (trifluoromethyl) phenyl)acetamide | 411.46 |
| 14 | 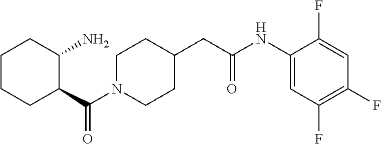 | trans-2-(1-(2- aminocyclohexanecarbonyl) piperidin-4- yl)-N-(2,4,5- trifluorophenyl)acetamide | 397.43 |
| 15 | 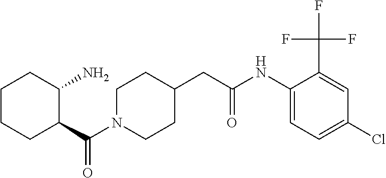 | trans-2-(1-(2- aminocyclohexanecarbonyl) piperidin-4- yl)-N-(4-chloro-2- (trifluoromethyl) phenyl)acetamide | 445.91 |
| 16 | 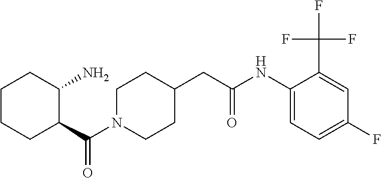 | trans-2-(1-(2- aminocyclohexanecarbonyl) piperidin-4- yl)-N-(4-fluoro-2- (trifluoromethyl) phenyl)acetamide | 429.45 |
| 17 | 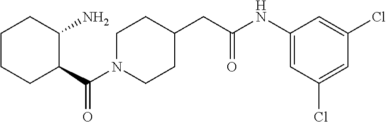 | trans-2-(1-(2- aminocyclohexanecarbonyl) piperidin-4- yl)-N-(3,5- dichlorophenyl)acetamide | 412.35 |
| 18 | 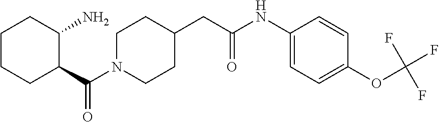 | trans-2-(1-(2- aminocyclohexanecarbonyl) piperidin-4- yl)-N-(4- (trifluoromethoxy) phenyl)acetamide | 427.46 |
| 19 | 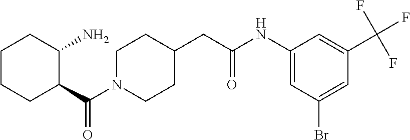 | trans-2-(1-(2- aminocyclohexanecarbonyl) piperidin-4- yl)-N-(3-bromo-5- (trifluoromethyl) phenyl)acetamide | 490.36 |
| 20 | 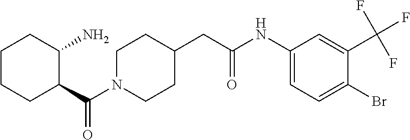 | trans-2-(1-(2- aminocyclohexanecarbonyl) piperidin-4- yl)-N-(4-bromo-3- (trifluoromethyl) phenyl)acetamide | 490.36 |
| 21 | 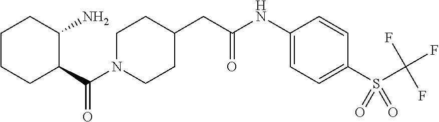 | trans-2-(1-(2- aminocyclohexanecarbonyl) piperidin-4- yl)-N-(4- (trifluoromethylsulfonyl) phenyl)acetamide | 475.52 |
| 22 | 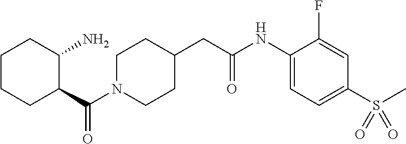 | trans-2-(1-(2- aminocyclohexanecarbonyl) piperidin-4- yl)-N-(2-fluoro-4- (methylsulfonyl) phenyl)acetamide | 439.54 |
| 23 | 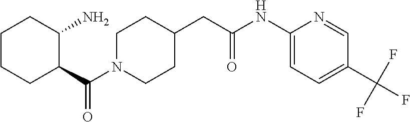 | trans-2-(1-(2- aminocyclohexanecarbonyl) piperidin-4- yl)-N-(5- (trifluoromethyl) pyridin-2-yl)acetamide | 412.45 |
| 24 | 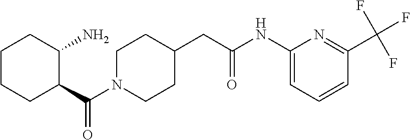 | trans-2-(1-(2- aminocyclohexanecarbonyl) piperidin-4- yl)-N-(6- (trifluoromethyl) pyridin-2-yl)acetamide | 412.45 |
| 25 | 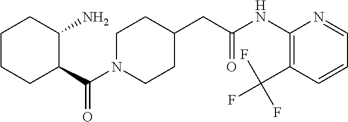 | trans-2-(1-(2- aminocyclohexanecarbonyl) piperidin-4- yl)-N-(3- (trifluoromethyl) pyridin-2-yl)acetamide | 412.45 |
| 26 | 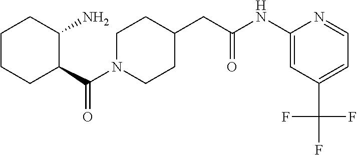 | trans-2-(1-(2- aminocyclohexanecarbonyl) piperidin-4- yl)-N-(4- (trifluoromethyl) pyridin-2-yl)acetamide | 412.45 |
| 27 | 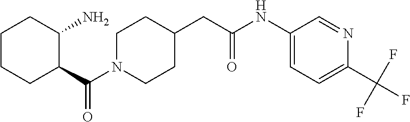 | trans-2-(1-(2- aminocyclohexanecarbonyl) piperidin-4- yl)-N-(6- (trifluoromethyl) pyridin-3-yl)acetamide | 412.45 |
| 28 | 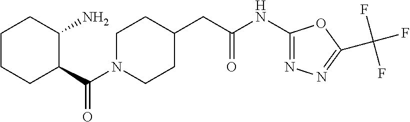 | trans-2-(1-(2- aminocyclohexanecarbonyl) piperidin-4- yl)-N-(5- (trifluoromethyl)- 1,3,4-oxadiazol-2- yl)acetamide | 403.40 |
| 29 | 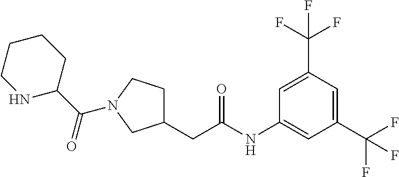 | N-(3,5- bis(trifluoromethyl) phenyl)-2-(1- (piperidine-2- carbonyl)pyrrolidin-3- yl)acetamide | 451.41 |
| 30 | 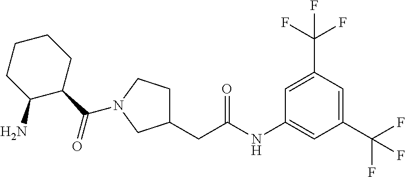 | 2-(1-((1R,2S)-2- aminocyclohexanecarbonyl) pyrrolidin-3- yl)-N-(3,5- bis(trifluoromethyl) phenyl)acetamide | 465.43 |
| 31 | 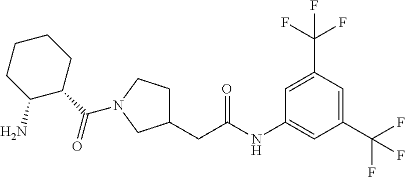 | 2-(1-((1S,2R)-2- aminocyclohexanecarbonyl) pyrrolidin-3- yl)-N-(3,5- bis(trifluoromethyl) phenyl)acetamide | 465.43 |
| 32 | 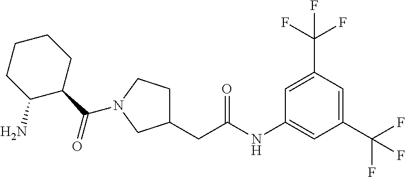 | 2-(1-((1R,2R)-2- aminocyclohexanecarbonyl) pyrrolidin-3- yl)-N-(3,5- bis(trifluoromethyl) phenyl)acetamide | 465.43 |
| 33 | 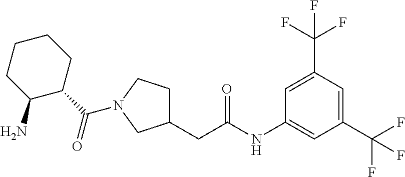 | 2-(1-((1S,2S)-2- aminocyclohexanecarbonyl) pyrrolidin-3- yl)-N-(3,5- bis(trifluoromethyl) phenyl)acetamide | 465.43 |
| 34 | 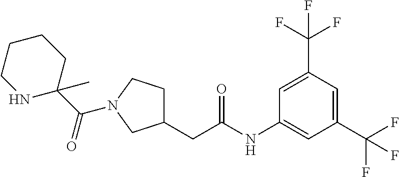 | N-(3,5- bis(trifluoromethyl) phenyl)-2-(1-(2- methylpiperidine-2- carbonyl)pyrrolidin-3- yl)acetamide | 465.43 |
| 35 | 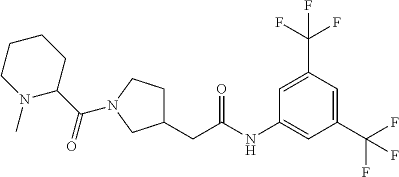 | N-(3,5- bis(trifluoromethyl) phenyl)-2-(1-(1- methylpiperidine-2- carbonyl)pyrrolidin-3- yl)acetamide | 465.43 |
| 36 | 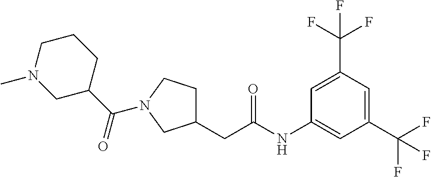 | N-(3,5- bis(trifluoromethyl) phenyl)-2-(1-(1- methylpiperidine-3- carbonyl)pyrrolidin-3- yl)acetamide | 465.43 |
| 37 | 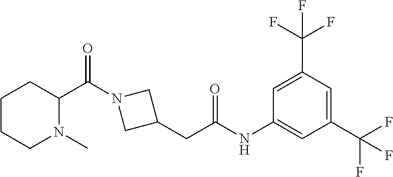 | N-(3,5- bis(trifluoromethyl) phenyl)-2-(1-(1- methylpiperidine-2- carbonyl)azetidin-3- yl)acetamide | 451.41 |
| 38 | 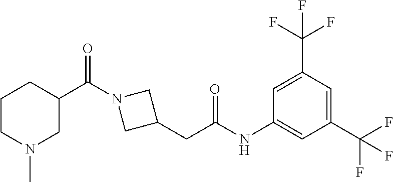 | N-(3,5- bis(trifluoromethyl) phenyl)-2-(1-(1- methylpiperidine-3- carbonyl)azetidin-3- yl)acetamide | 451.41 |
| 39 | 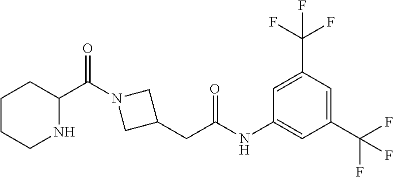 | N-(3,5- bis(trifluoromethyl) phenyl)-2-(1- (piperidine-2- carbonyl)azetidin-3- yl)acetamide | 437.38 |
| 40 | 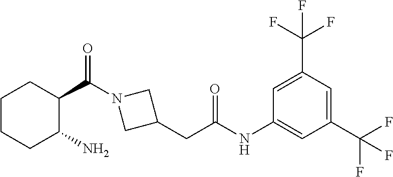 | trans-2-(1-(2- aminocyclohexanecarbonyl) azetidin-3-yl)- N-(3,5- bis(trifluoromethyl) phenyl)acetamide | 451.41 |
| 41 | 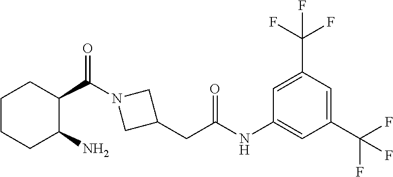 | cis-2-(1-(2- aminocyclohexanecarbonyl) azetidin-3-yl)- N-(3,5- bis(trifluoromethyl) phenyl)acetamide | 451.41 |
| 42 | 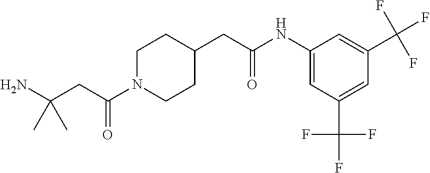 | 2-(1-(3-amino-3- methylbutanoyl) piperidin-4-yl)-N-(3,5- bis(trifluoromethyl) phenyl)acetamide | 453.42 |
| 43 | 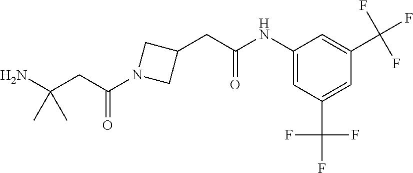 | 2-(1-(3-amino-3- methylbutanoyl) azetidin-3-yl)-N-(3,5- bis(trifluoromethyl) phenyl)acetamide | 425.37 |
| 44 | 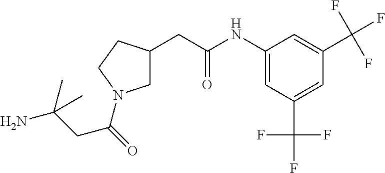 | 2-(1-(3-amino-3- methylbutanoyl) pyrrolidin-3-yl)-N-(3,5- bis(trifluoromethyl) phenyl)acetamide | 439.40 |
| 45 | 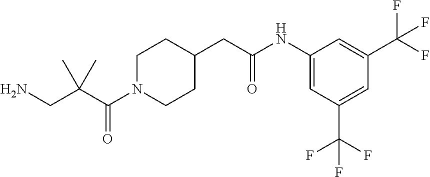 | 2-(1-(3-amino-2,2- dimethylpropanoyl) piperidin-4-yl)-N-(3,5- bis(trifluoromethyl) phenyl)acetamide | 453.42 |
| 46 | 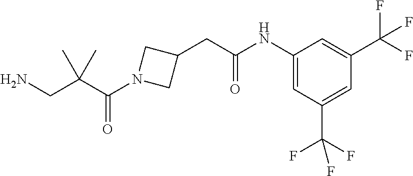 | 2-(1-(3-amino-2,2- dimethylpropanoyl) azetidin-3-yl)-N-(3,5- bis(trifluoromethyl) phenyl)acetamide | 425.37 |
| 47 | 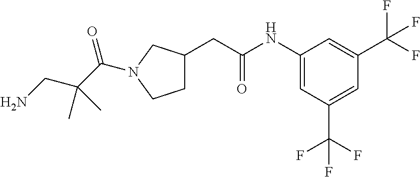 | 2-(1-(3-amino-2,2- dimethylpropanoyl) pyrrolidin-3-yl)-N-(3,5- bis(trifluoromethyl) phenyl)acetamide | 439.40 |
| 48 | 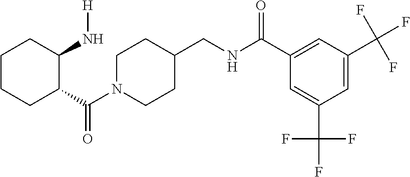 | trans-2-(1-(2- aminocyclohexanecarbonyl) piperidin-4- yl)methyl)-3,5- bis(trifluoromethyl) benzamide | 479.46 |
| 49 | 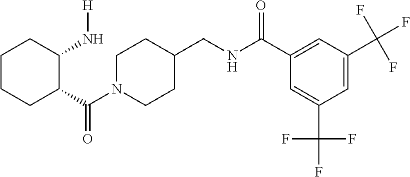 | cis-2-(1-(2- aminocyclohexanecarbonyl) piperidin-4- yl)methyl)-3,5- bis(trifluoromethyl) benzamide | 479.46 |
| 50 | 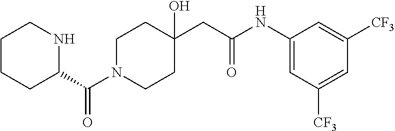 | (S)-N-(3,5- bis(trifluoromethyl) phenyl)-2-(4-hydroxy-1- (piperidine-2- carbonyl)piperidin-4- yl)acetamide | 481.432 |
| 51 | 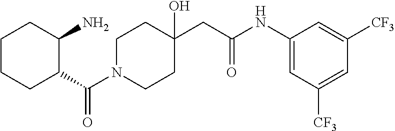 | trans-2-(1-(2- aminocyclohexanecarbonyl)- 4- hydroxypiperidin-4- yl)-N-(3,5- bis(trifluoromethyl) phenyl)acetamide | 495.458 |
| 52 | 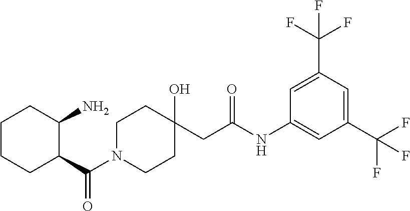 | cis-2-(1-(2- aminocyclohexanecarbonyl)- 4- hydroxypiperidin-4- yl)-N-(3,5- bis(trifluoromethyl) phenyl)acetamide | 495.458 |
| 53 | 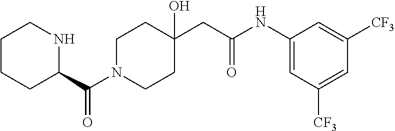 | (R)-N-(3,5- bis(trifluoromethyl) phenyl)-2-(4-hydroxy-1- (piperidine-2- carbonyl)piperidin-4- yl)acetamide | 481.432 |
| 54 | 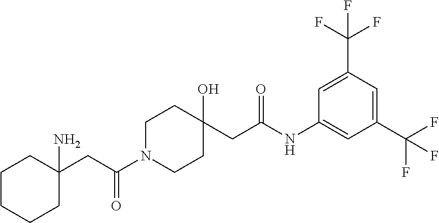 | 2-(1-(2-(1- aminocyclohexyl) acetyl)-4- hydroxypiperidin-4- yl)-N-(3,5- bis(trifluoromethyl) phenyl)acetamide | 509.485 |
| 55 | 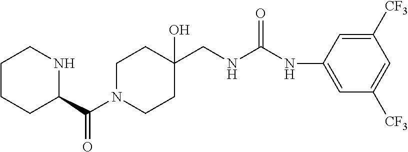 | (S)-1-(3,5- bis(trifluoromethyl) phenyl)-3-((1- (piperidine-2- carbonyl)piperidin-4- yl)methyl)urea | 480.447 |
| 56 | 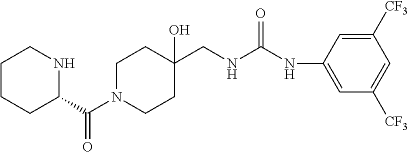 | (R)-1-(3,5- bis(trifluoromethyl) phenyl)-3-((1- (piperidine-2- carbonyl)piperidin-4- yl)methyl)urea | 480.447 |
| 57 | 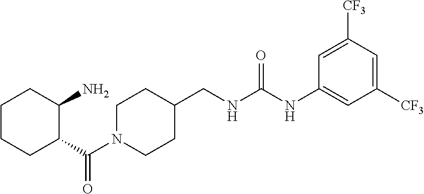 | trans-2-(1-(2- aminocyclohexanecarbonyl) piperidin-4- yl)methyl)-3-(3,5- bis(trifluoromethyl) phenyl)urea | 494.474 |
| 58 | 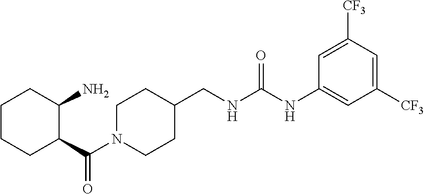 | cis-2-(1-(2- aminocyclohexanecarbonyl) piperidin-4- yl)methyl)-3-(3,5- bis(trifluoromethyl) phenyl)urea | 494.474 |
| 59 | 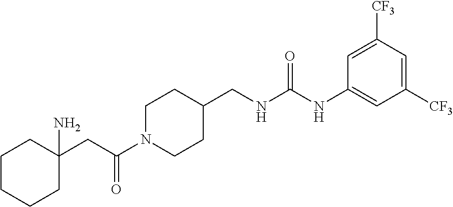 | 1-((1-(2-(1- aminocyclohexyl) acetyl)piperidin-4- yl)methyl)-3-(3,5- bis(trifluoromethyl) phenyl)urea | 508.5 |
| 60 | 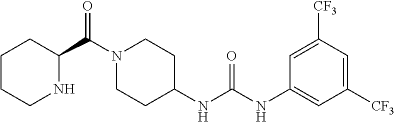 | (S)-1-(3,5- bis(trifluoromethyl) phenyl)-3-(1- (piperidine-2- carbonyl)piperidin-4- yl)urea | 466.421 |
| 61 | 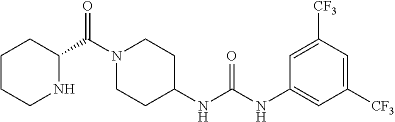 | (R)-1-(3,5- bis(trifluoromethyl) phenyl)-3-(1- (piperidine-2- carbonyl)piperidin-4- yl)urea | 466.421 |
| 62 | 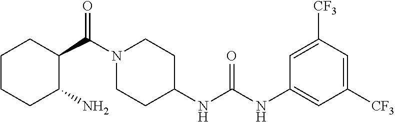 | trans-2-(1-(2- aminocyclohexanecarbonyl) piperidin-4- yl)-3-(3,5- bis(trifiuoromethyl) phenyl)urea | 480.447 |
| 63 | 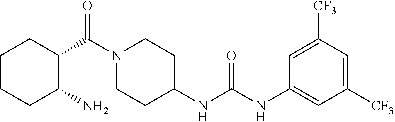 | cis-2-(1-(2- aminocyclohexanecarbonyl) piperidin-4- yl)-3-(3,5- bis(trifluoromethyl) phenyl)urea | 480.447 |
| 64 | 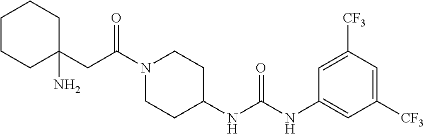 | 1-(1-(2-(1- aminocyclohexyl) acetyl)piperidin-4-yl)-3- (3,5- bis(trifluoromethyl) phenyl)urea | 494.474 |
| 65 | 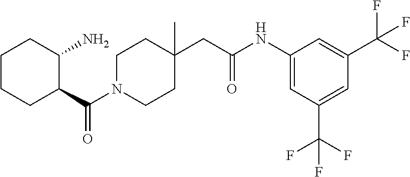 | | |
| 66 | 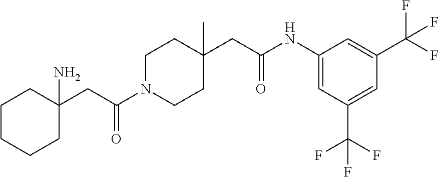 | | |
| 67 | 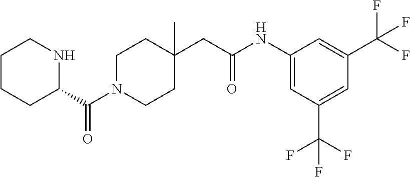 | | |
| 68 | 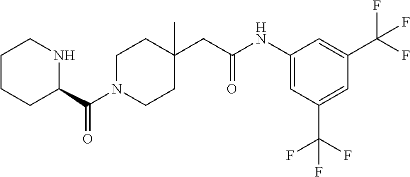 | | |
| 69 | 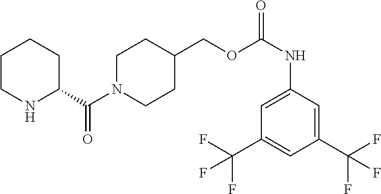 | | |
| 70 | 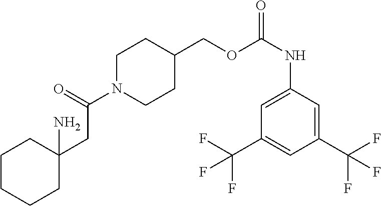 | | |
| 71 | 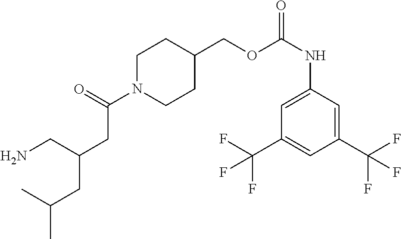 | | |
| 72 | 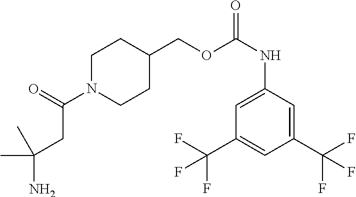 | | |
| 73 | 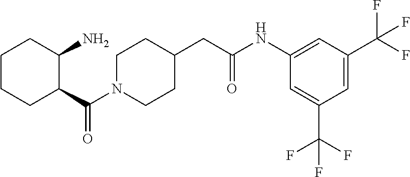 | | |
| 74 | 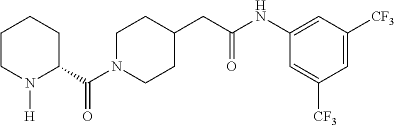 | | |
| 75 | 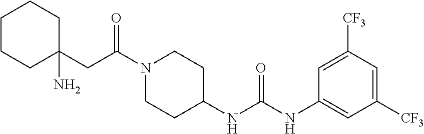 | | |

### Example 15

### T-Type Channel Blocking Activities

A. Transformation of HEK Cells:

T-type calcium channel blocking activity was assayed in human embryonic kidney cells, HEK 293 (Invitrogen), stably transfected with the T-type calcium channel subunits. Briefly, cells were cultured in Dulbecco's modified eagle medium (DMEM) supplemented with 10% fetal bovine serum, 200 U/ml penicillin, and 0.2 mg/mL streptomycin at 37° C. with 5% CO₂. At 85% confluency, cells were split with 0.25% trypsin/1 mM EDTA and plated at 10% confluency on glass coverslips. At 12 hours, the medium was replaced, and the cells stably transfected using a standard calcium phosphate protocol and the appropriate calcium channel cDNA's. Fresh DMEM was supplied, and the cells transferred to 28° C./5% CO₂. Cells were incubated for 1 to 2 days prior to whole cell recording.

Standard patch-clamp techniques were employed to identify blockers of T-type currents. Briefly, previously described HEK cell lines stably expressing human α_{1G}, α_{1H} and α_{1I} T-type channels were used for all the recordings (passage #: 4-20, 37° C., 5% CO₂). Whole cell patch clamp experiments were performed using an Axopatch 200B amplifier (Axon Instruments, Burlingame, Calif.) linked to a personal computer equipped with pCLAMP software. Data were analyzed using Clampfit (Axon Instruments) and SigmaPlot 4.0 (Jandel Scientific). To obtain T-type currents, plastic dishes containing semi-confluent cells were positioned on the stage of a ZEISS AXIOVERT S100 microscope after replacing the culture medium with external solution (Table 2). Whole-cell patches were obtained using pipettes (borosilicate glass with filament, O.D.: 1.5 mm, I.D.: 0.86 mm, 10 cm length), fabricated on a SUTTER P-97 puller with resistance values of ˜5 MΩ (Table 3).

TABLE 2
External Solution 500 ml - pH 7.4, 265.5 mOsm

| Salt | Final mM | Stock M | Final ml |
|---|---|---|---|
| CsCl | 142 | 1 | 71 |
| CaCl₂ | 2 | 1 | 1 |
| MgCl₂ | 1 | 1 | 0.5 |
| HEPES | 10 | 0.5 | 10 |
| glucose | 10 | — | 0.9 grams |

TABLE 3
Internal Solution 50 ml - pH 7.3 with CsOH, 270 mOsm

| Salt | Final mM | Stock M | Final ml |
|---|---|---|---|
| Cs-Methanesulfonate | 126.5 | — | 1.442 gr/50 ml |
| MgCl2 | 2 | 1 | 0.1 |
| HEPES | 10 | 0.5 | 1 |
| EGTA-Cs | 11 | 0.25 | 2.2 |
| ATP | 2 | 0.2 | 0.025 |
| | | | (1 aliquot/2.5 ml) |

T-type currents were reliably obtained by using two voltage protocols:

“non-inactivating,” and

“inactivation.”

In the non-inactivating protocol, the holding potential is set at −110 mV and with a pre-pulse at −100 mV for 1 second prior to the test pulse at −40 mV for 50 ms. In the inactivation protocol, the pre-pulse is at approximately −85 mV for 1 second, which inactivates about 15% of the T-type channels (Scheme 1).

Test compounds were dissolved in external solution, 0.1-0.01% DMSO. After ˜10 minutes rest, they were applied by gravity close to the cell using a WPI microfil tubing. The “non-inactivated” pre-pulse was used to examine the resting block of a compound. The “inactivated” protocol was employed to study voltage-dependent block. However, the initial data shown below were mainly obtained using the non-inactivated protocol only. IC₅₀ values are shown for various compounds of the invention in Table 4. Values are shown in nM, with values above 10,000 nM represented as “10000 nM.” The data show that each of compounds 1-3, 5-9, 17, 19, 20, 29-33, 40-42, 45, 50-52, 54 and 56-64 exhibited activity at less than 1 μM. Further, compounds 3, 54, and 59 exhibited activity at less than 0.01 μM, with compound 59 demonstrating the lowest IC₅₀. In Table 4, empty cells indicate that inhibition by the compound was not detected.

TABLE 4
T-type Calcium Channel Block

| | α_{1G} (Ca_{V} 3.1) | α_{1H} (Ca_{V} 3.2) |
|---|---|---|
| Compound | nM | nM |
| 1 | 10000 | 510 |
| 2 | | 350 |
| 3 | 10000 | 60 |
| 4 | 10000 | 2180 |
| 5 | 10000 | 100 |
| 6 | 10000 | 250 |
| 7 | 10000 | 140 |
| 8 | 10000 | 110 |
| 9 | 4680 | 990 |
| 10 | | |
| 11 | | 10000 |
| 12 | | |
| 13 | 10000 | 2670 |
| 14 | | 10000 |
| 15 | | |
| 16 | | |
| 17 | 10000 | 690 |
| 18 | | 2730 |
| 19 | 10000 | 280 |
| 20 | 10000 | 920 |
| 21 | | |
| 22 | | |
| 23 | | |
| 24 | | |
| 25 | | |
| 26 | | |
| 27 | | |
| 28 | | |
| 29 | 7020 | 570 |
| 30 | 8950 | 710 |
| 31 | 6190 | 460 |
| 32 | 5660 | 630 |
| 33 | 5290 | 260 |
| 34 | 4400 | 1940 |
| 35 | 6020 | 1950 |
| 36 | 8100 | 2440 |
| 37 | 10000 | 4390 |
| 38 | 10000 | 8040 |
| 39 | 10000 | 1210 |
| 40 | 10000 | 830 |
| 41 | 10000 | 850 |
| 42 | 10000 | 570 |
| 43 | 10000 | 10000 |
| 44 | 10000 | 2100 |
| 45 | 10000 | 540 |
| 46 | 10000 | 10000 |
| 47 | 10000 | 3370 |
| 48 | 10000 | 1850 |
| 49 | | 1930 |
| 50 | | 520 |
| 51 | | 490 |
| 52 | | 820 |
| 53 | | 1860 |
| 54 | | 80 |
| 55 | | 1110 |
| 56 | | 770 |
| 57 | | 250 |
| 58 | | 250 |
| 59 | | 30 |
| 60 | | 950 |
| 61 | | 690 |
| 62 | | 270 |
| 63 | | 430 |
| 64 | | 360 |

### Example 16

### L5/L6 Spinal Nerve Ligation (SNL)—Chung Pain Model

The Spinal Nerve Ligation is an animal model representing peripheral nerve injury generating a neuropathic pain syndrome. In this model experimental animals develop the clinical symptoms of tactile allodynia and hyperalgesia. L5/L6 Spinal nerve ligation (SNL) injury was induced using the procedure of Kim and Chung (Kim et al., Pain 50:355-363 (1992)) in male Sprague-Dawley rats (Harlan; Indianapolis, Ind.) weighing 200 to 250 grams.

Anaesthesia was induced with 2% isofluorane in O₂ at 2 L/min and maintained with 0.5% isofluorane in O₂. Rats were then shaved and aseptically prepared for surgeries. A 2 cm paraspinal incision was made at the level of L4-S2. L4/L5 was exposed by removing the transverse process above the nerves with a small rongeur. The L5 spinal nerve is the larger of the two visible nerves below the transverse process and lies closest to the spine. The L6 spinal nerve is located beneath the corner of the slope bone. A home-made glass Chung rod was used to hook L5 or L6 and a pre-made slip knot of 4.0 silk suture was placed on the tip of the rod just above the nerve and pulled underneath to allow for the tight ligation. The L5 and L6 spinal nerves were tightly ligated distal to the dorsal root ganglion. The incision was closed, and the animals were allowed to recover for 5 days. Rats that exhibited motor deficiency (such as paw-dragging) or failure to exhibit subsequent tactile allodynia were excluded from further testing.

Sham control rats underwent the same operation and handling as the experimental animals, but without SNL.

Prior to initiating drug delivery, baseline behavioural testing data is obtained. At selected times after infusion of the Test or Control Article behavioural data can then be collected again.

### A. Assessment of Tactile Allodynia—Von Frey

The assessment of tactile allodynia consisted of measuring the withdrawal threshold of the paw ipsilateral to the site of nerve injury in response to probing with a series of calibrated von Frey filaments (innocuous stimuli). Animals were acclimated to the suspended wire-mesh cages for 30 min before testing. Each von Frey filament was applied perpendicularly to the plantar surface of the ligated paw of rats for 5 sec. A positive response was indicated by a sharp withdrawal of the paw. For rats, the first testing filament is 4.31. Measurements were taken before and after administration of test articles. The paw withdrawal threshold was determined by the non-parametric method of Dixon (Dixon, Ann. Rev. Pharmacol. Toxicol. 20:441-462 (1980)), in which the stimulus was incrementally increased until a positive response was obtained, and then decreased until a negative result was observed. The protocol was repeated until three changes in behaviour were determined (“up and down” method) (Chaplan et al., J. Neurosci. Methods 53:55-63 (1994)). The 50% paw withdrawal threshold was determined as (10^{[Xf+kδ]})/10,000, where X_{f}=the value of the last von Frey filament employed, k=Dixon value for the positive/negative pattern, and δ=the logarithmic difference between stimuli. The cut-off values for rats were no less than 0.2 g and no higher than 15 g (5.18 filament); for mice no less than 0.03 g and no higher than 2.34 g (4.56 filament). A significant drop of the paw withdrawal threshold compared to the pre-treatment baseline is considered tactile allodynia. Table 5 shows exemplary rat SNL data obtained for Compounds 2, 54, and 75.

TABLE 5

| | | Rat SNL tactile allodynia | | |
|---|---|---|---|---|
| | | (% antiallodynia) | | |
| No. | Structure | 1 hr | 2 hr | 4 hr |
| 2 | 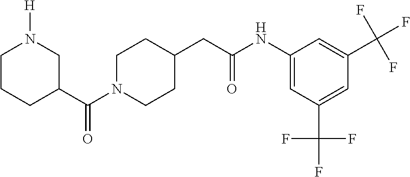 | 38 | 44 | 27 |
| 54 | 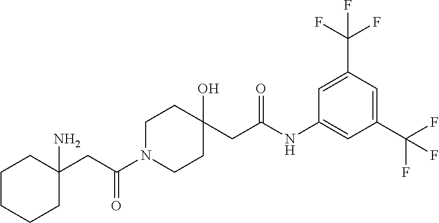 | 41 | 39 | 45 |
| 75 | 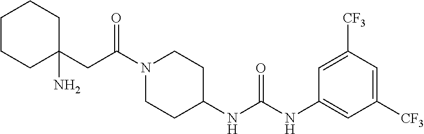 | 5 | 3 | 3 |

Compound No. 3 from Table 1 was also tested in rats using this allodynia model, with the results as shown in Table 6. In these studies, gabapentin was dosed at 100 mg/kg orally in water. When compared to gabapentin as a control, Compound 3, at a dosage of 30 mg/kg, showed slightly lower efficacy at the 2 hr time point than gabapentin at a dosage of 100 mg/kg.

TABLE 6

| | % Antiallodynia (Mean ± SD) (n = 7-8) | | |
|---|---|---|---|
| | Gabapentin | Compound 3 | Vehicle |
| Time | (100 mg/kg PO) | (30 mg/kg, PO) | (DMSO/PEG) |
| 1 h | 73 ± 35 #* | 36 ± 19 #* | 1 ± 11 |
| 2 h | 90 ± 18 #* | 75 ± 26 #* | 1 ± 1 |
| 4 h | 15 ± 8 | 4 ± 8 | 0 ± 0 |

# - p < 0.05 compared to pre-dose baseline
* p < 0.05 compared to vehicle alone at same time point

In a second repetition of this study at a later date than the one reported above, gabapentin performed similarly and compound 3 produced anti-allodynia effects of 68±12% at 1 hr, 76±6% at 2 hr, and 42±14% at 4 hr (n=9).

### B. Assessment of Thermal Hypersensitivity—Hargreaves

The method of Hargreaves and colleagues (Hargreaves et al., Pain 32:77-8 (1988)) can be employed to assess paw-withdrawal latency to a noxious thermal stimulus.

Rats may be allowed to acclimate within a Plexiglas enclosure on a clear glass plate for 30 minutes. A radiant heat source (e.g., halogen bulb coupled to an infrared filter) can then be activated with a timer and focused onto the plantar surface of the affected paw of treated rats. Paw-withdrawal latency can be determined by a photocell that halts both lamp and timer when the paw is withdrawn. The latency to withdrawal of the paw from the radiant heat source can be determined prior to L5/L6 SNL, 7-14 days after L5/L6 SNL but before drug, as well as after drug administration. A maximal cut-off of 33 seconds is typically employed to prevent tissue damage. Paw withdrawal latency can be thus determined to the nearest 0.1 second. A significant drop of the paw withdrawal latency from the baseline indicates the status of thermal hyperalgesia. Antinociception is indicated by a reversal of thermal hyperalgesia to the pre-treatment baseline or a significant (p<0.05) increase in paw withdrawal latency above this baseline. Data is converted to % anti hyperalgesia or % anti nociception by the formula: (100×(test latency−baseline latency)/(cut-off−baseline latency) where cut-off is 21 seconds for determining anti hyperalgesia and 40 seconds for determining anti nociception.

### Example 17

### 6 Hz Psychomotor Seizure Model of Partial Epilepsy

Compounds can also be evaluated for the protection against seizures induced by a 6 Hz, 0.2 ms rectangular pulse width of 3 s duration, at a stimulus intensity of 32 mA (CC97) applied to the cornea of male CF1 mice (20-30 g) according to procedures described by Barton et al, “Pharmacological Characterization of the 6 Hz Psychomotor Seizure Model of Partial Epilepsy,” Epilepsy Res. 47(3):217-27 (2001). Seizures are characterised by the expression of one or more of the following behaviours: stun, forelimb clonus, twitching of the vibrissae and Straub-tail immediately following electrical stimulation. Animals can be considered “protected” if following pre-treatment with a compound the 6 Hz stimulus failed to evoke a behavioural response as describe above. Exemplary data are shown in Table 7 below.

TABLE 7

| | | Epilepsy 6 Hz | | | | |
|---|---|---|---|---|---|---|
| | | (% Protected) | | | | |
| Cmpd | | 0.25 | 0.5 | 1 | 2 | 4 |
| no. | Structure | hr | hr | hr | hr | hr |
| 2 | 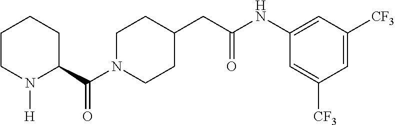 | 50 | 50 | 100 | 33 | 0 |
| 3 | 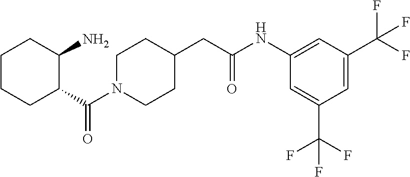 | 100 | 75 | 100 | 0 | 50 |
| 8 | 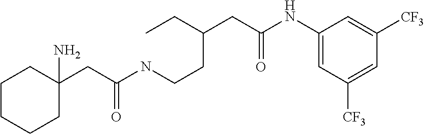 | 75 | 75 | 67 | 100 | |
| 54 | 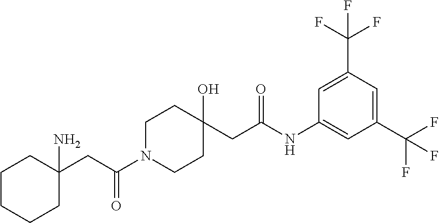 | 25 | 75 | 75 | 75 | 100 |
| 59 | 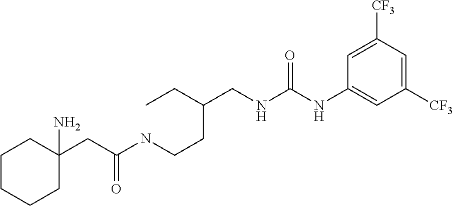 | 25 | 50 | 100 | | |
| 68 | 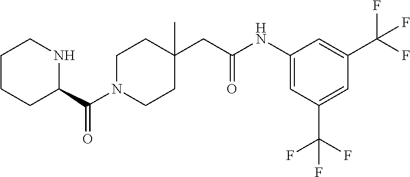 | 50 | 75 | 100 | 50 | 0 |
| 69 | 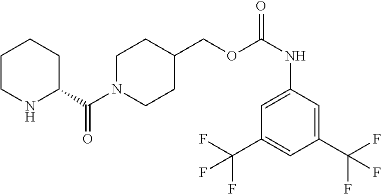 | 75 | 75 | 50 | 100 | 33 |
| 70 | 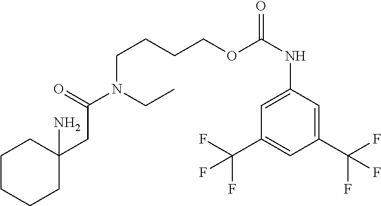 | 0 | 75 | 100 | | |
| 73 | 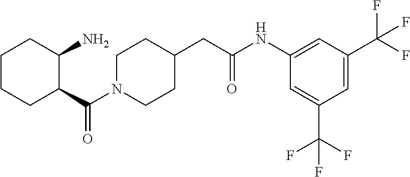 | 100 | 100 | 100 | | 100 |

### Example 18

### Mouse Rotarod Assay

To assess a compound's undesirable side effects (toxicity), animals can be monitored for overt signs of impaired neurological or muscular function. In mice, the rotarod procedure (Dunham and Miya, J. Am. Pharmacol. Assoc. 46:208-209 (1957)) is used to disclose minimal muscular or neurological impairment (MMI). When a mouse is placed on a rod that rotates at a speed of 6 rpm, the animal can maintain its equilibrium for long periods of time. The animal is considered toxic if it falls off this rotating rod three times during a 1-min period. In addition to MMI, animals may exhibit a circular or zigzag gait, abnormal body posture and spread of the legs, tremors, hyperactivity, lack of exploratory behavior, somnolence, stupor, catalepsy, loss of placing response and changes in muscle tone. Exemplary data are shown in Table 8 below.

TABLE 8

| | | Rotarod Assay | | | | |
|---|---|---|---|---|---|---|
| | | (% impaired) | | | | |
| Cmpd | | 0.25 | 0.5 | 1 | 2 | 4 |
| No. | Structure | hr | hr | hr | hr | hr |
| 2 | 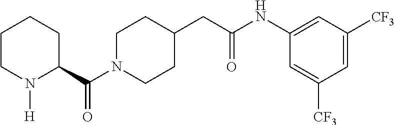 | 25 | 50 | 100 | 100 | 50 |
| 3 | 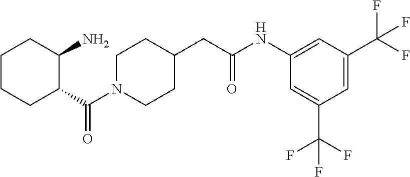 | 0 | 75 | 100 | 75 | 50 |
| 8 | 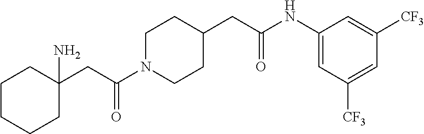 | 0 | 50 | 75 | 100 | 100 |
| 4 | 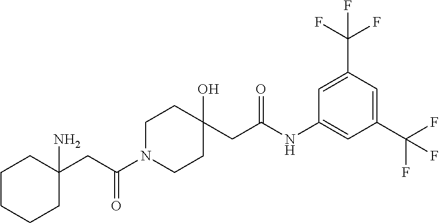 | 25 | 0 | 75 | 75 | 75 |
| 59 | 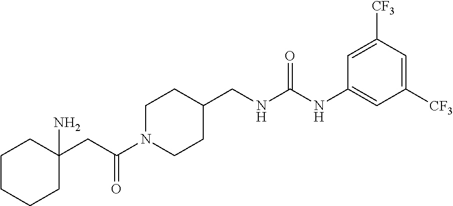 | 0 | 25 | 75 | 100 | 100 |
| 69 | 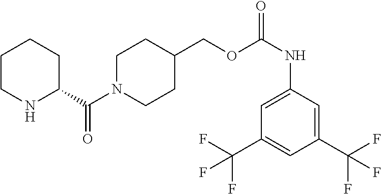 | 0 | 50 | 50 | 100 | 100 |
| 70 | 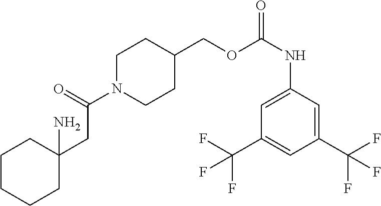 | 0 | 0 | 100 | 100 | 100 |
| 73 | 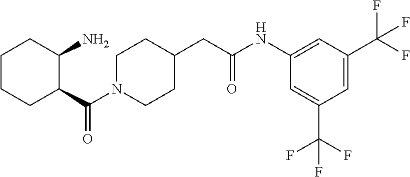 | 100 | 100 | 100 | 100 | 75 |

### Example 19

### Lamina Assay and Data

### Recordings on Lamina I/II Spinal Cord Neurons.

Male Wistar rats (P6 to P9 for voltage-clamp and P15 to P18 for current-clamp recordings) were anaesthetized through intraperitoneal injection of Inactin (Sigma). The spinal cord was then rapidly dissected out and placed in an ice-cold solution protective sucrose solution containing (in mM): 50 sucrose, 92 NaCl, 15 D-Glucose, 26 NaHCO₃, 5 KCl, 1.25 NaH₂PO₄, 0.5 CaCl₂, 7 MgSO₄, 1 kynurenic acid, and bubbled with 5% CO₂/95% O₂. The meninges, dura, and dorsal and ventral roots were then removed from the lumbar region of the spinal cord under a dissecting microscope. The “cleaned” lumbar region of the spinal cord was glued to the vibratome stage and immediately immersed in ice cold, bubbled, sucrose solution. For current-clamp recordings, 300 to 350 μm parasagittal slices were cut to preserve the dendritic arbour of lamina I neurons, while 350 to 400 μm transverse slices were prepared for voltage-clamped Nay channel recordings. Slices were allowed to recover for 1 hour at 35° C. in Ringer solution containing (in mM): 125 NaCl, 20 D-Glucose, 26 NaHCO₃, 3 KCl, 1.25 NaH₂PO₄, 2 CaCl₂, 1 MgCl₂, 1 kynurenic acid, 0.1 picrotoxin, bubbled with 5% CO₂/95% O₂. The slice recovery chamber was then returned to room temperature (20 to 22° C.) and all recordings were performed at this temperature.

Neurons were visualized using IR-DIC optics (Zeiss Axioskop 2 FS plus, Gottingen, Germany), and neurons from lamina I and the outer layer of lamina II were selected based on their location relative to the substantia gelatinosa layer. Neurons were patch-clamped using borosilicate glass patch pipettes with resistances of 3 to 6 ma Current-clamp recordings of lamina I/II neurons in the intact slice, the external recording solution was the above Ringer solution, while the internal patch pipette solution contained (in mM): 140 KGluconate, 4 NaCl, 10 HEPES, 1 EGTA, 0.5 MgCl₂, 4 MgATP, 0.5 Na₂GTP, adjusted to pH 7.2 with 5 M KOH and to 290 mOsm with D-Mannitol (if necessary). Only tonic firing neurons were selected for current-clamp experiments, while phasic, delayed onset and single spike neurons were discarded (22). Recordings were digitized at 50 kHz and low-pass filtered at 2.4 kHz.

Data obtained according to this protocol are shown in Table 9.

TABLE 9

| | | LAMINA I AND II | | | |
|---|---|---|---|---|---|
| Cmpd. | | % Spike Change | | | |
| no. | Structure | Mean | SEM | P < 0.05? | EC₅₀ |
| 2 | 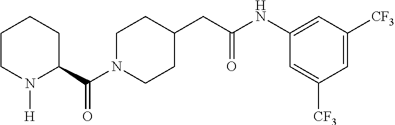 | −57.2 | 6.3 | no | 6550 |
| 3 | 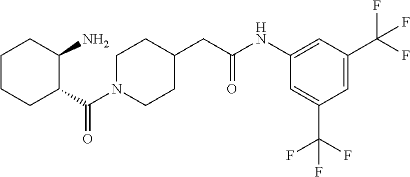 | −51.1 | 13.2 | yes | |
| 74 | 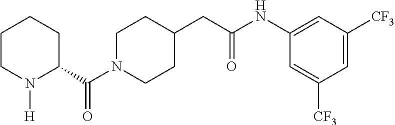 | −53 | 11.8 | yes | |

### Other Embodiments

While the invention has been described in connection with specific embodiments thereof, it will be understood that it is capable of further modifications and this application is intended to cover any variations, uses, or adaptations of the invention following, in general, the principles of the invention and including such departures from the present disclosure come within known or customary practice within the art to which the invention pertains and may be applied to the essential features hereinbefore set forth.

All publications, patents and patent applications are herein incorporated by reference in their entirety to the same extent as if each individual publication, patent or patent application was specifically and individually indicated to be incorporated by reference in its entirety.

1. A compound according to the following formula, 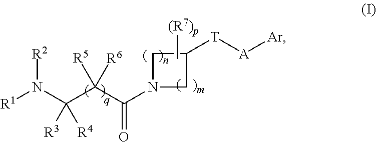
or a pharmaceutically acceptable salt, solvate, or prodrug thereof, or a stereoisomer thereof, or a conjugate thereof, wherein
Ar is phenyl or a 5-6 membered heteroaryl ring containing at least one heteroatom selected from N, O and S as a ring member, and optionally substituted with at least one R⁸, R⁹, or R¹⁰;
T is CH₂, O, or NR¹;
A is [T]-C(O)—NR¹ or [T]-NR¹—C(O)— or [T]-C(O)—O—, or [T]-O—C(O)—, —NR¹—C(O)—NR¹—; [T]-O—C(O)—NR¹—; or [T]-NR¹—C(O)—O—; wherein [T] indicates which atom of A is linked to T in Formula (I);
R¹ and R² are independently selected from the group consisting of H, optionally substituted C1-C6 alkyl, C1-C6 optionally substituted alkylsulfonyl, and optionally substituted C1-C6 acyl;
R³, R⁴, R⁵, R⁶ and R⁷ are independently selected from H, optionally substituted C1-C6 alkyl, halo, hydroxy, CN, optionally substituted C1-C6 alkoxy, and optionally substituted C1-C6 heteroalkyl;
wherein R² and R³, or R³ and R⁴, or R⁴ and R⁵, or R⁶ and R², or two R⁶ if two R⁶ are present (any one of these pairs, but not more than one pair) can optionally be taken together to form a non-aromatic 5-6 membered ring, which can optionally include up to two heteroatoms selected from N, O and S as ring members;
and two R⁷ can optionally be taken together to form a non-aromatic 5-6 membered ring, which can optionally include a heteroatom selected from N, O and S as a ring member
m and n are independently 1 or 2;
p is 0-2;
q is 0, 1 or 2;
R⁸, R⁹, and R¹⁰ are optional substituents that are independently selected from the group consisting of H, halogen, CN, —SO₂—(C1-C4 alkyl), optionally substituted C1-C6 alkyl, optionally substituted C1-C6 alkoxy, optionally substituted aryl, and optionally substituted heteroaryl, provided at least one of R⁸, R⁹, and R¹⁰ is present and is not H.

2. The compound of claim 1, wherein said compound has a structure according to the following formula, 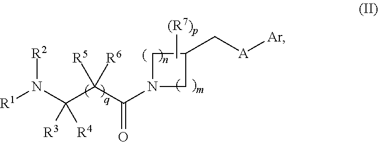
wherein
q is 0 or 1;
A is an amide of the formula —NR¹—C(O)— or —C(O)—NR¹—; or a urea of the formula —NR¹—C(O)—NR¹—; or a carbamate of the formula —O—C(O)—NR¹— or —NR¹—C(O)—O—;
Ar is a phenyl or pyridyl group that is substituted by R⁸, R⁹, and R¹⁰, or Ar is a 1,3,4-oxadiazolyl group optionally substituted by R⁸; and
R⁸, R⁹, and R¹⁰ are, independently, selected from the group consisting of H, F, Cl, Br, CN, optionally substituted C1-C6 alkyl, optionally substituted C1-C6 alkoxy, and —SO₂-(optionally substituted C1-C4 alkyl).

3. (canceled)

4. The compound of claim 1, wherein
R⁸, R⁹, and R¹⁰ are, independently, selected from F, Cl, Br, Me, OMe, SO₂CF₃, —OCF₃, —OCHF₂, C2-C4 alkyl, C2-C4 alkoxy, —CHF₂, —CH₂F, and —CF₃; or
m is 2 and n is either 1 or 2; or
p is 0; or
R⁴ and R⁵ taken together form a non-aromatic 5-6 membered ring, which can optionally include a heteroatom selected from N, O and S as a ring member.

5.-10. (canceled)

11. The compound of claim 1, wherein said compound has a structure according to the following formula, 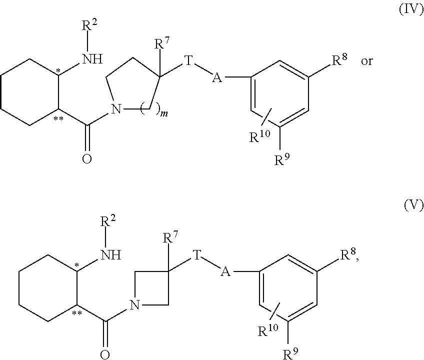
wherein
m is 1 or 2;
-T-A- is —CH₂CONH—, —CH₂NHCO—, —CH₂NHCONH—, —CH₂OCONH—, —NHCONH—, or —OCONH—;
R² is H or optionally substituted C1-C6 alkyl;
R⁷ is H, OH, or optionally substituted C1-C6 alkyl;
R⁸ is halogen or optionally substituted C1-C6 alkyl; and
R⁹ and R¹⁰ are, independently, selected from H, halogen, and optionally substituted C1-C6 alkyl.

12.-19. (canceled)

20. The compound of claim 1, wherein said compound has a structure according to the following formula, 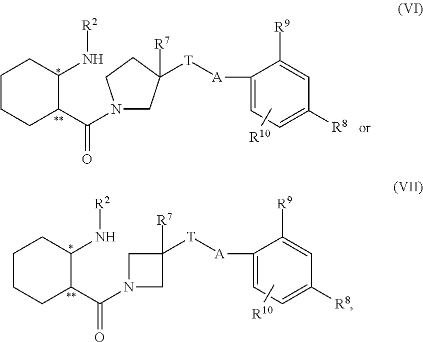
wherein
m is 1 or 2;
-T-A- is —CH₂CONH—, —CH₂NHCO—, —CH₂NHCONH—, —CH₂OCONH—, —NHCONH—, or —OCONH—;
R² is H or optionally substituted C1-C6 alkyl;
R⁷ is H, OH, or optionally substituted C1-C6 alkyl;
R⁸ is halogen, optionally substituted C1-C6 alkyl, optionally substituted C1-C6 alkoxy, or SO₂(optionally substituted C1-C6 alkyl); and
R⁹ and R¹⁰ are, independently, selected from H, halogen, and optionally substituted C1-C6 alkyl.

21.-29. (canceled)

30. The compound of claim 1, wherein said compound has a structure according to 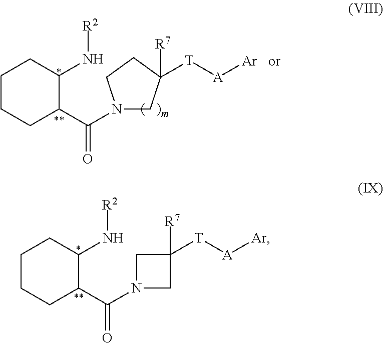
wherein
m is 1 or 2;
-T-A- is —CH₂CONH—, —CH₂NHCO—, —CH₂NHCONH—, —CH₂OCONH—, —NHCONH—, or —OCONH—;
R² is H or optionally substituted C1-C6 alkyl;
R⁷ is H, OH, or optionally substituted C1-C6 alkyl;
Ar is 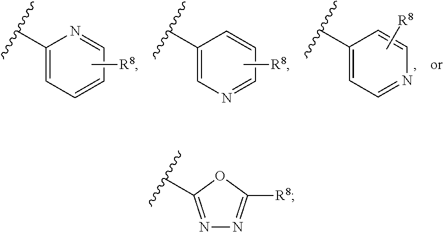
and
R⁸ is halogen, optionally substituted C1-C6 alkyl, optionally substituted C1-C6 alkoxy, or SO₂(optionally substituted C1-C6 alkyl).

31.-36. (canceled)

37. The compound of claim 1, wherein said compound has a structure according to the following formula, 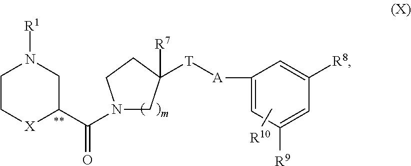
wherein
X is CH₂ or 0;
m is 1 or 2;
-T-A- is —CH₂CONH—, —CH₂NHCO—, —CH₂NHCONH—, —CH₂OCONH—, —NHCONH—, or —OCONH—;
R¹ is H or optionally substituted C1-C6 alkyl;
R² is H or optionally substituted C1-C6 alkyl;
R⁷ is H, OH, or optionally substituted C1-C6 alkyl;
R⁸ is halogen, optionally substituted C1-C6 alkyl, optionally substituted C1-C6 alkoxy, or SO₂(optionally substituted C1-C6 alkyl); and
R⁹ and R¹⁰ are, independently, selected from H, halogen, and optionally substituted C1-C6 alkyl.

38.-45. (canceled)

46. The compound of claim 1, wherein said compound has a structure according to the following formula, 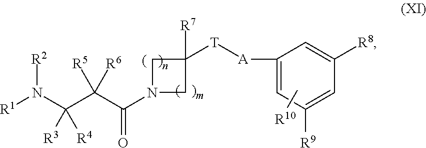
wherein
each of R¹-R⁶ is selected, independently, from H or unsubstituted C1-C6 alkyl;
n is 1 or 2;
m is 1 or 2;
-T-A- is —CH₂CONH—, —CH₂NHCO—, —CH₂NHCONH—, —CH₂OCONH—, —NHCONH—, or —OCONH—;
R⁷ is H, OH, or optionally substituted C1-C6 alkyl;
R⁸ is halogen, optionally substituted C1-C6 alkyl, optionally substituted C1-C6 alkoxy, or SO₂(optionally substituted C1-C6 alkyl); and
R⁹ and R¹⁰ are, independently, selected from H, halogen, and optionally substituted C1-C6 alkyl.

47.-53. (canceled)

54. The compound of claim 1, wherein said compound has a structure according to the following formula, 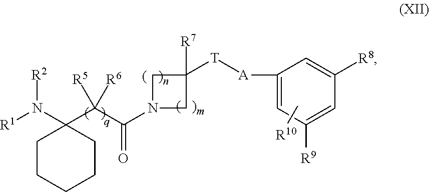
wherein
each of R¹, R², R⁵, R⁶, and R⁷ is selected, independently, from H or unsubstituted C1-C6 alkyl;
n is 1 or 2;
m is 1 or 2;
-T-A- is —CH₂CONH—, —CH₂NHCO—, —CH₂NHCONH—, —CH₂OCONH—, —NHCONH—, or —OCONH—;
R⁸ is halogen, optionally substituted C1-C6 alkyl, optionally substituted C1-C6 alkoxy, or SO₂(optionally substituted C1-C6 alkyl); and
R⁹ and R¹⁰ are, independently, selected from H, halogen, and optionally substituted C1-C6 alkyl.

55.-59. (canceled)

60. The compound of claim 1, wherein said compound has a structure according to the following formula, 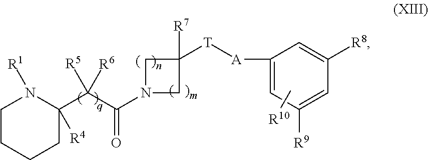
wherein
each of R¹, R⁴, R⁵, R⁶, and R⁷ is selected, independently, from H or unsubstituted C1-C6 alkyl;
n is 1 or 2;
m is 1 or 2;
-T-A- is —CH₂CONH—, —CH₂NHCO—, —CH₂NHCONH—, —CH₂OCONH—, —NHCONH—, or —OCONH—;
R⁸ is halogen, optionally substituted C1-C6 alkyl, optionally substituted C1-C6 alkoxy, or SO₂(optionally substituted C1-C6 alkyl); and
R⁹ and R¹⁰ are, independently, selected from H, halogen, and optionally substituted C1-C6 alkyl.

61.-65. (canceled)

66. The compound of claim 1, wherein said compound is selected from the group consisting of: 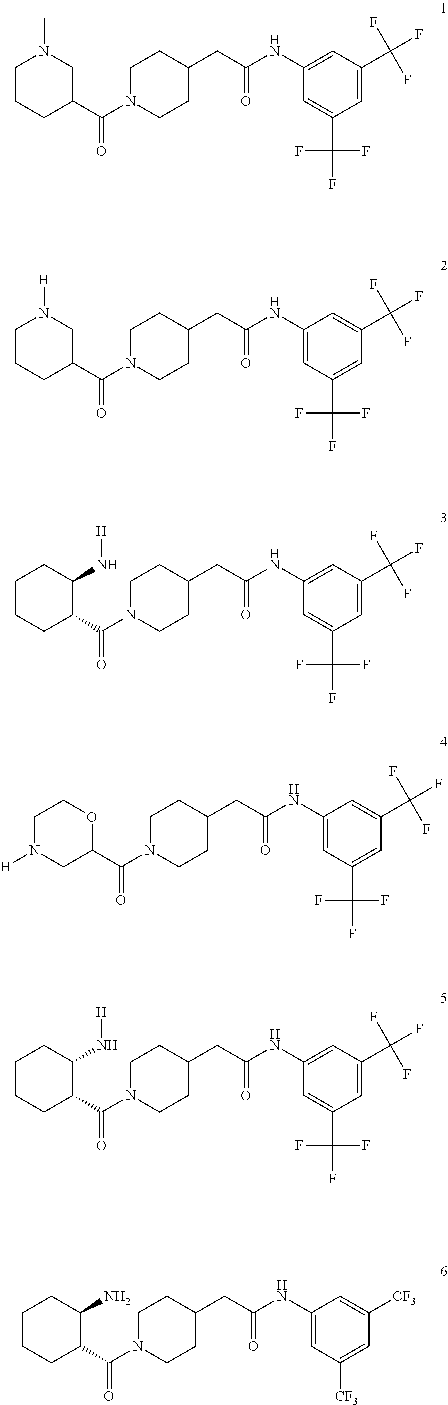 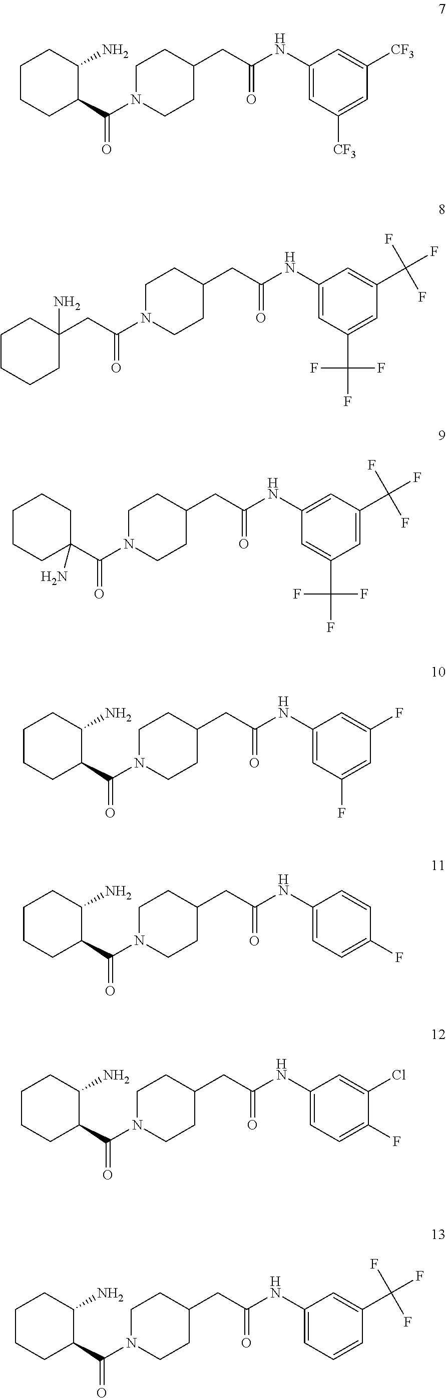 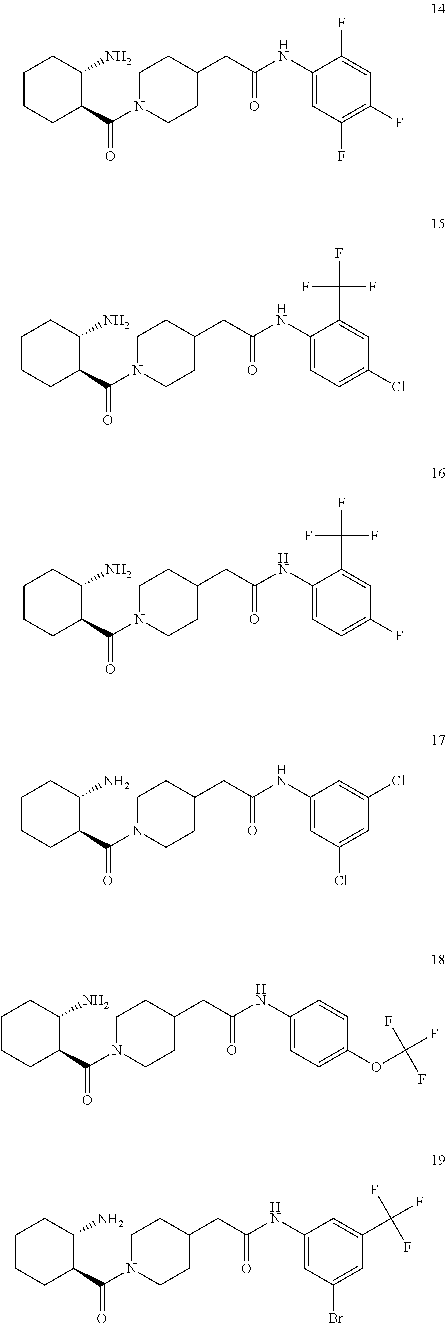 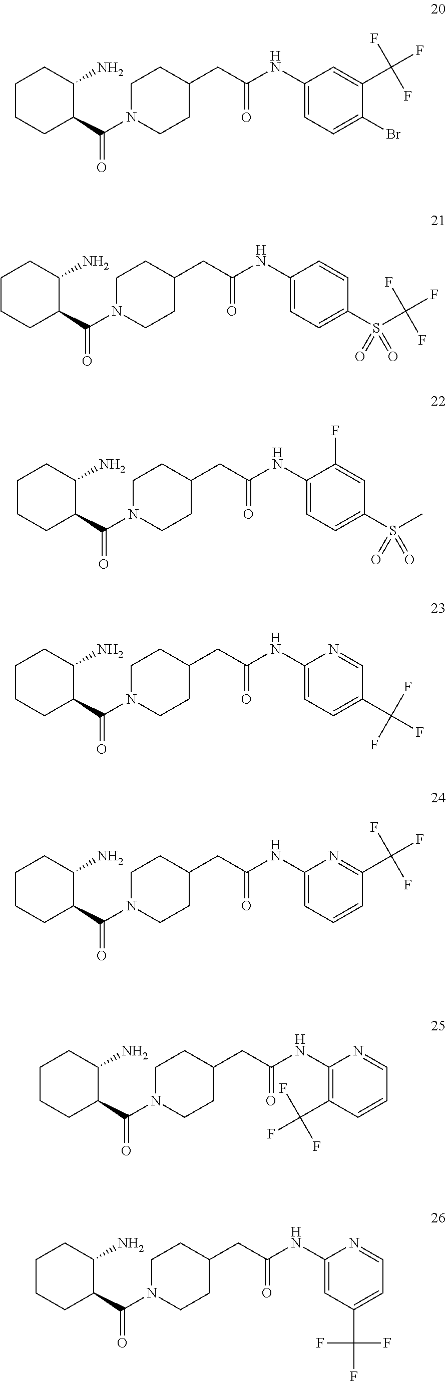 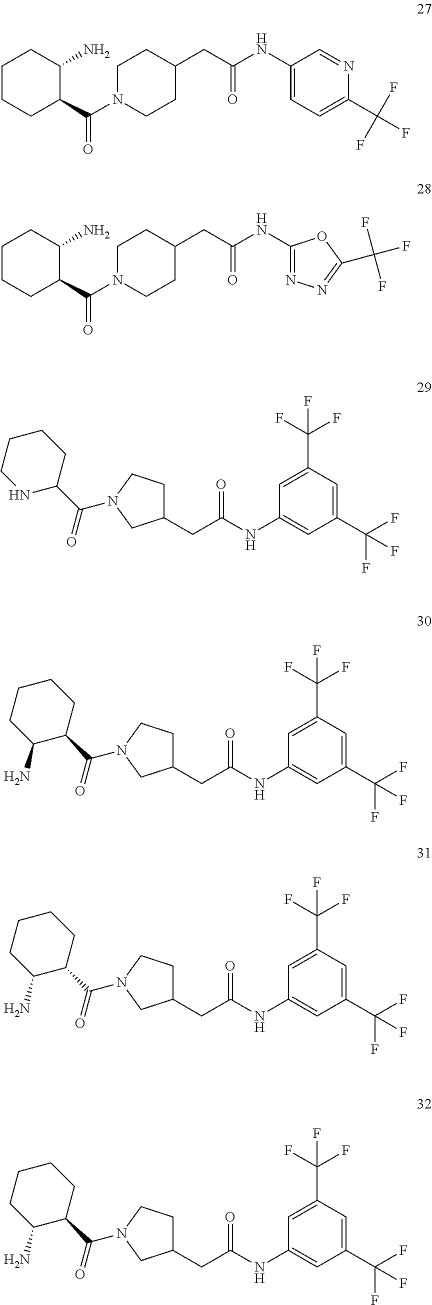 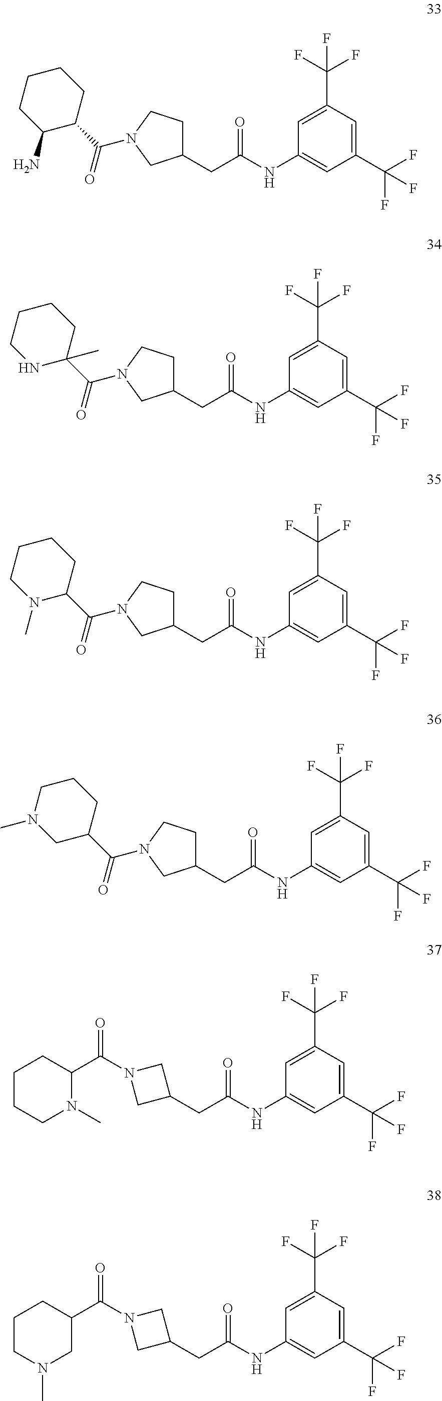 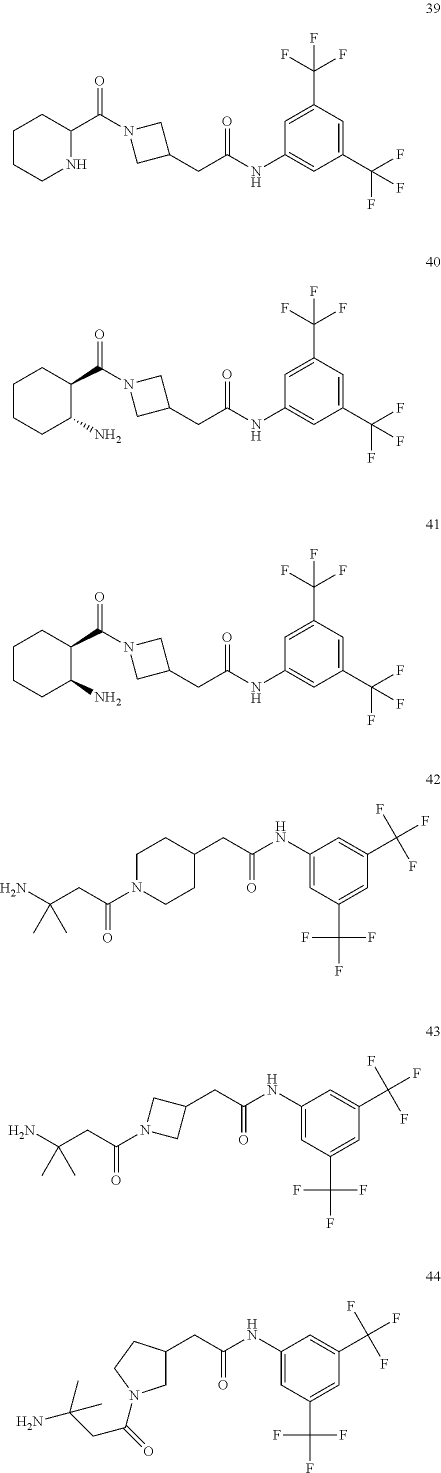 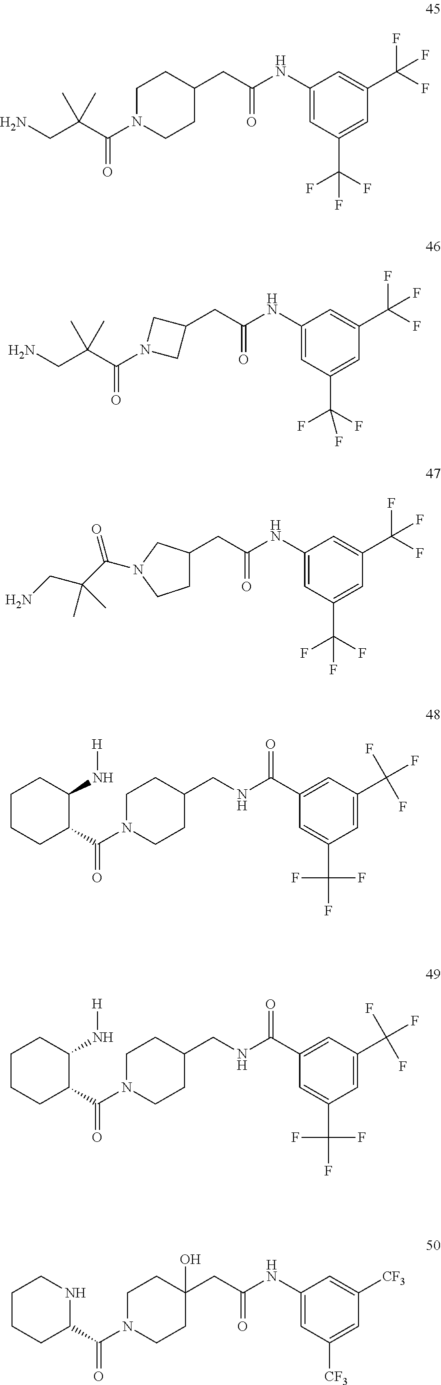 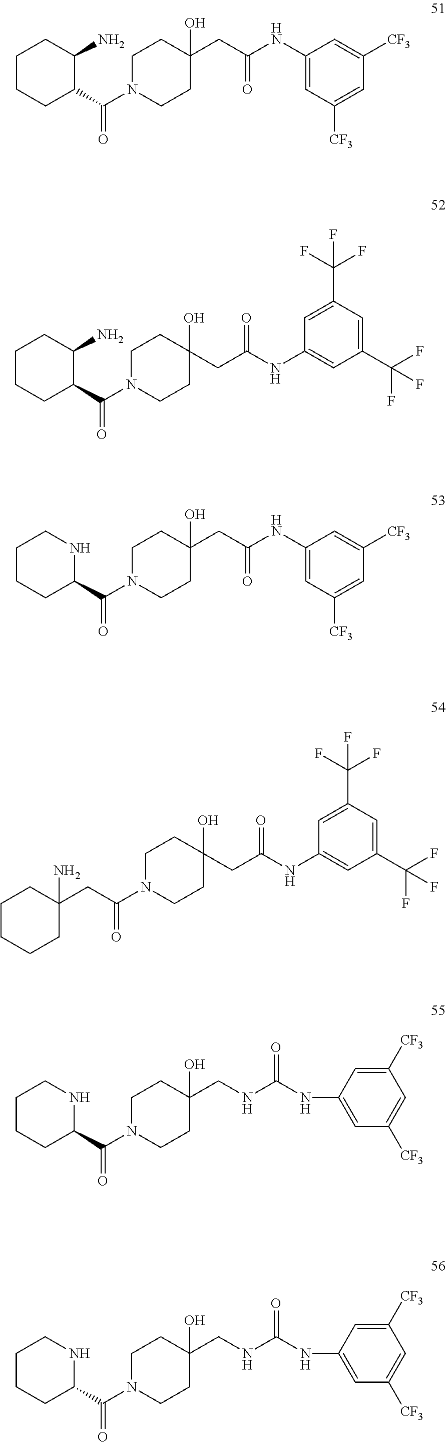 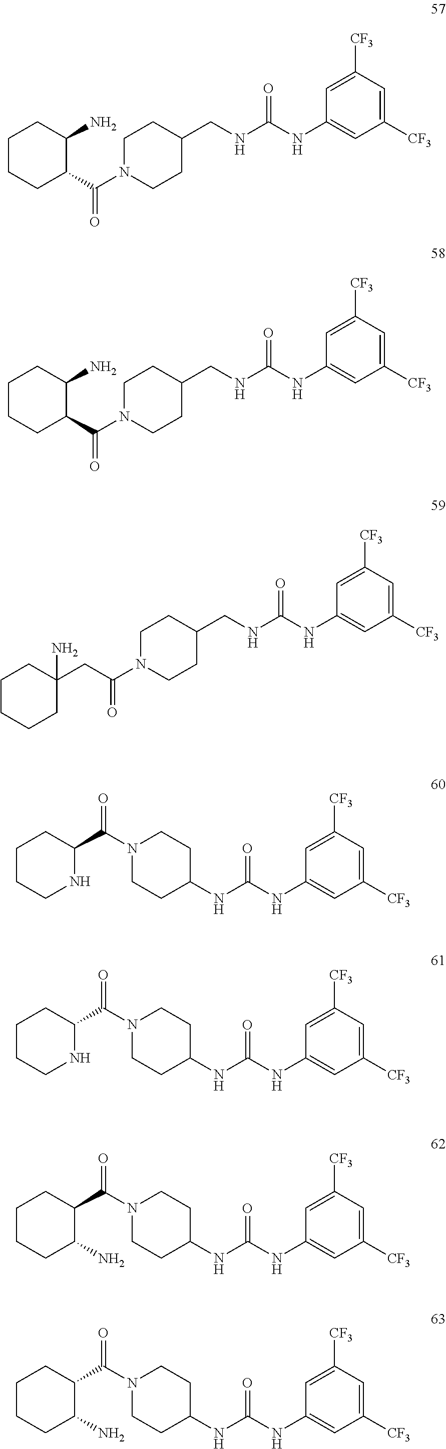 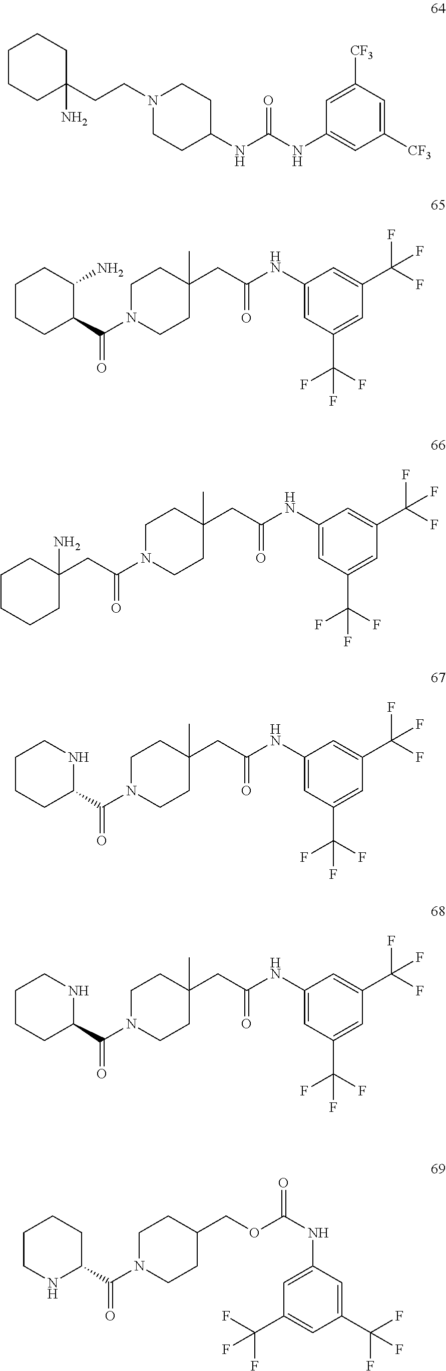 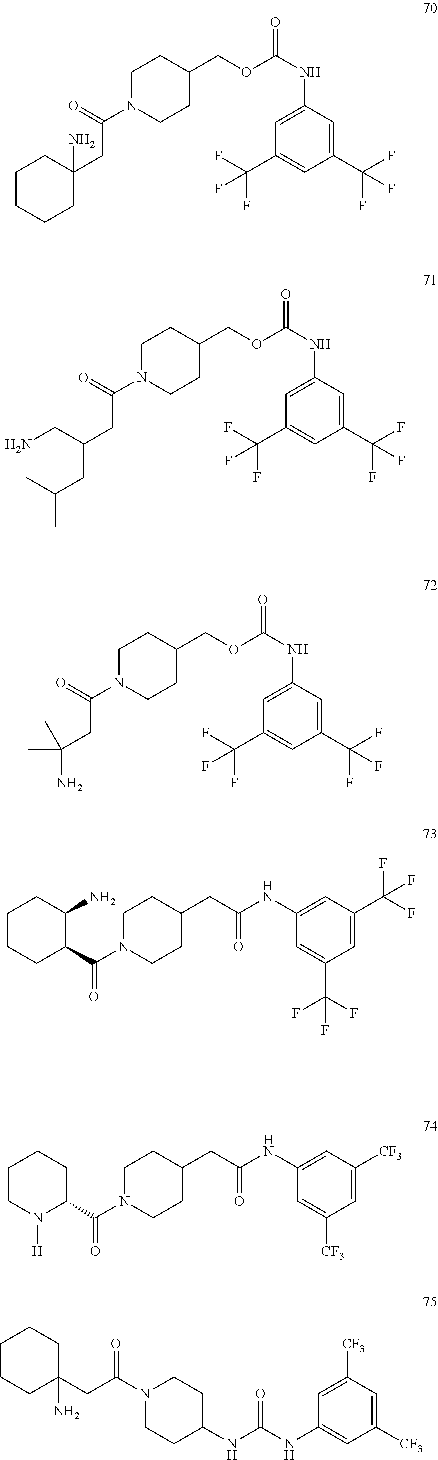
or a pharmaceutically acceptable salt, solvate, or prodrug thereof, or a stereoisomer thereof, or a conjugate thereof.

67. The compound of claim 66, wherein said compound is selected from the group consisting of: 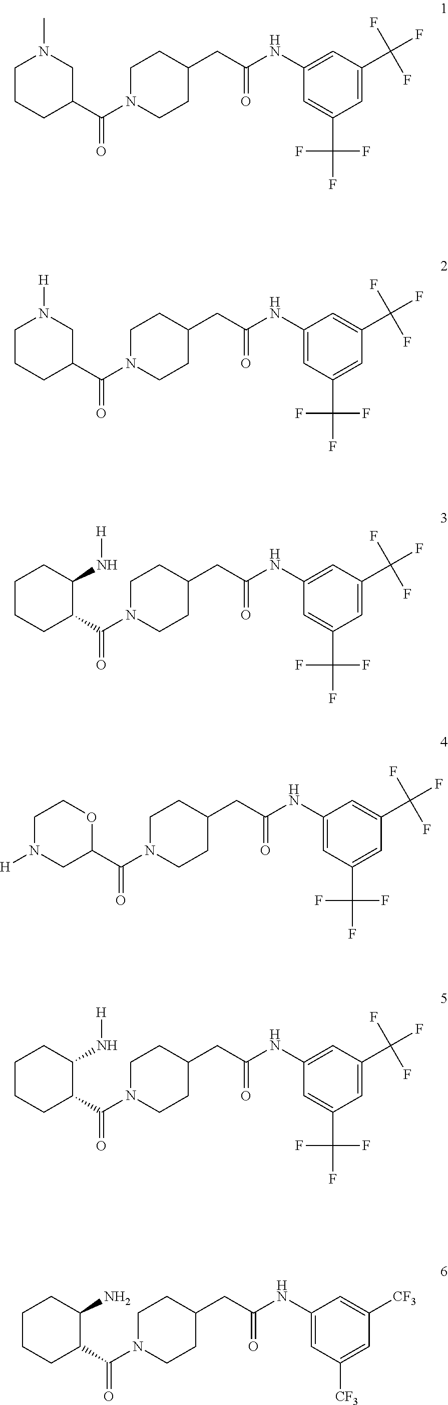 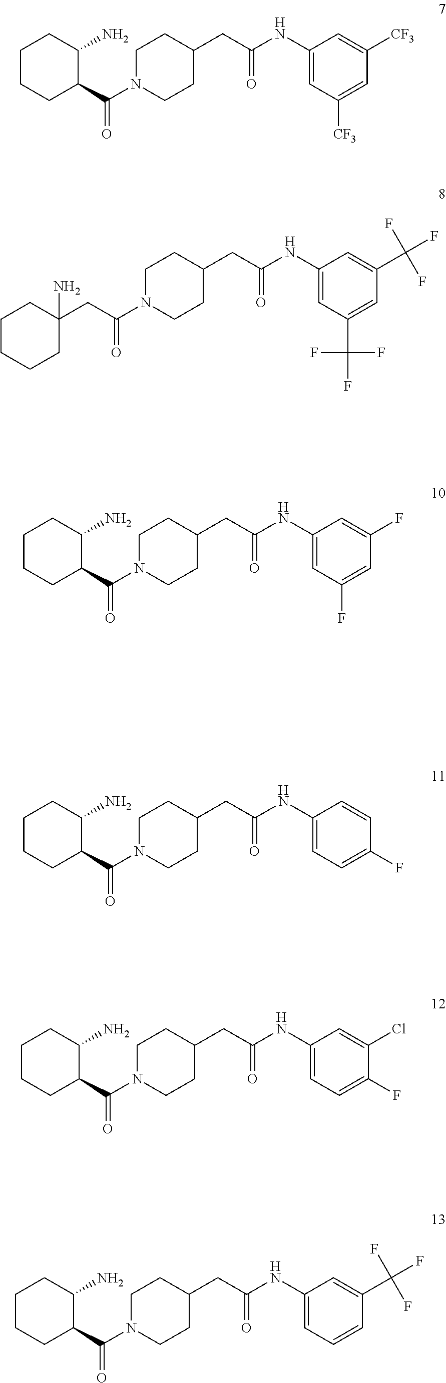 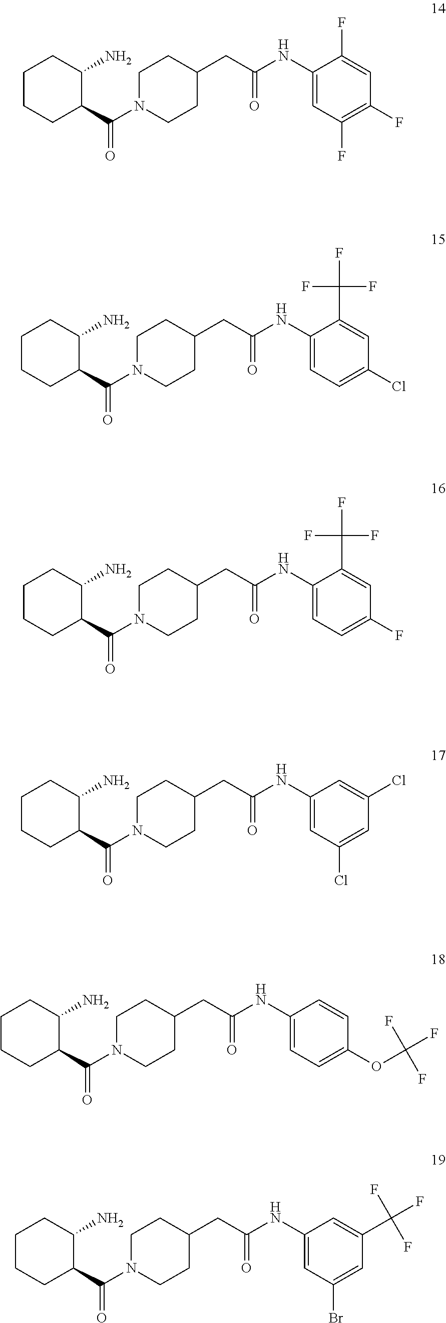 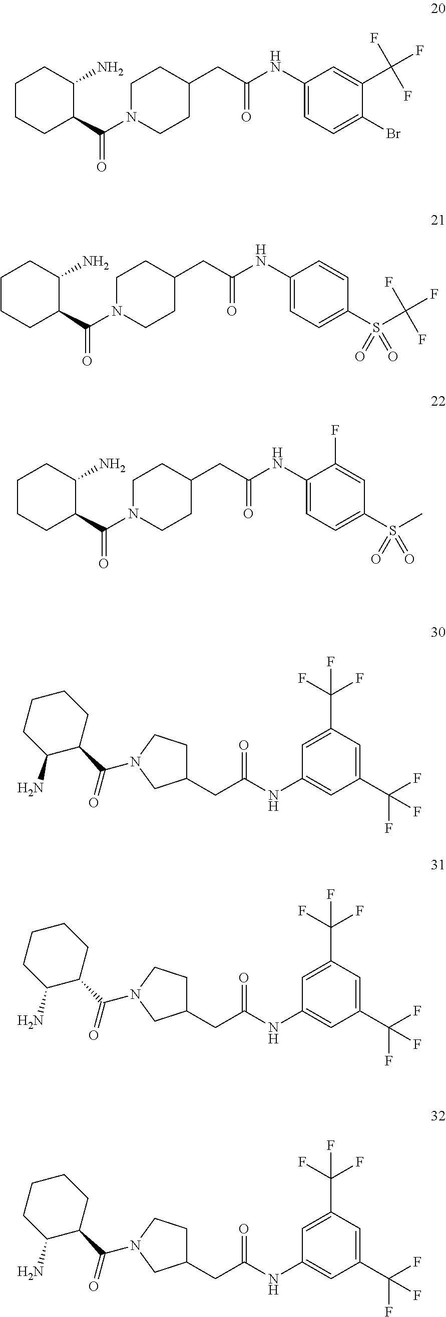 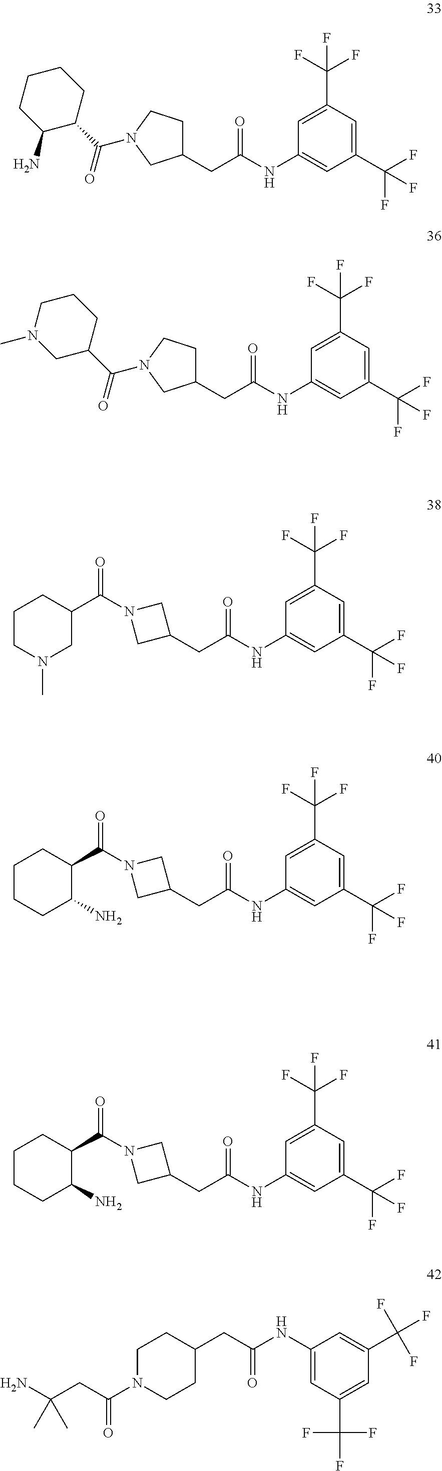 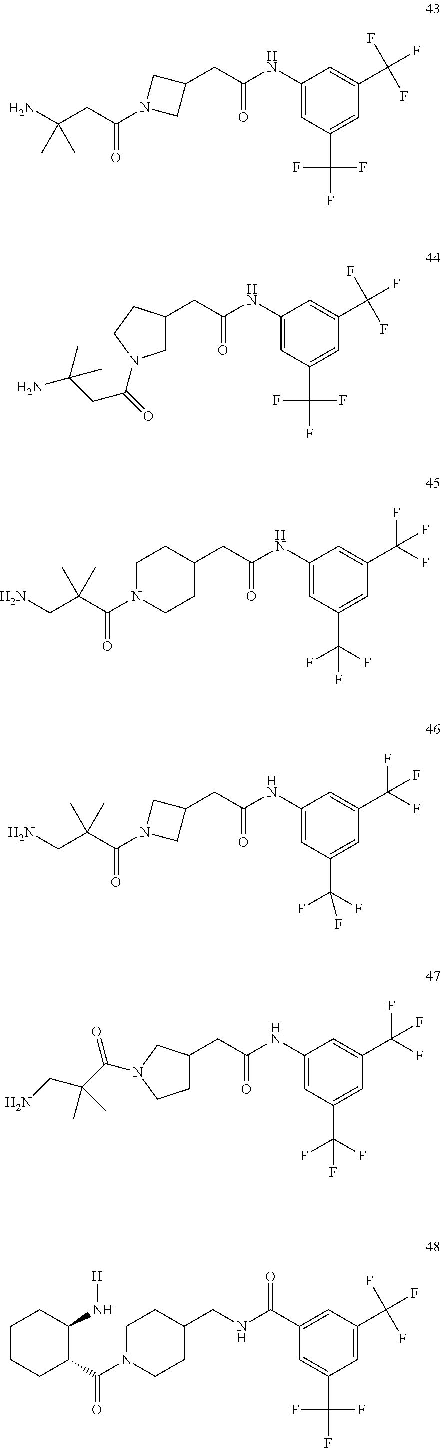 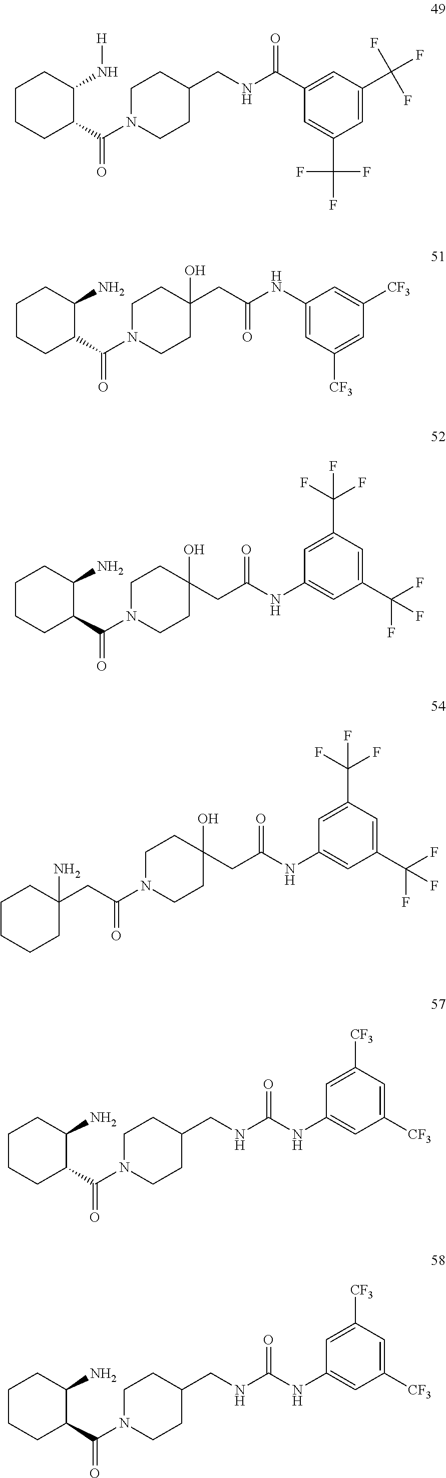 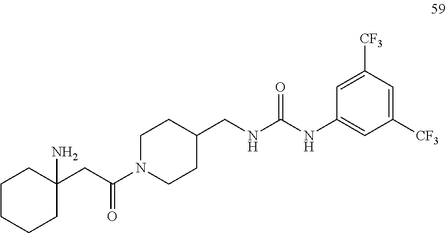
or a pharmaceutically acceptable salt, solvate, or prodrug thereof, or a stereoisomer thereof, or a conjugate thereof.

68. The compound of claim 67, wherein said compound is 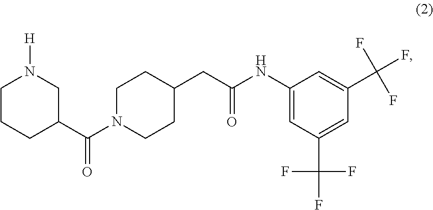
or a pharmaceutically acceptable salt, solvate, or prodrug thereof, or a stereoisomer thereof, or a conjugate thereof.

69. A pharmaceutical composition comprising the compound of claim 1, or a pharmaceutically acceptable salt, solvate, or prodrug thereof, or a stereoisomer thereof, or a conjugate thereof, and a pharmaceutically acceptable carrier or excipient.

70. The pharmaceutical composition of claim 69, wherein said pharmaceutical composition is formulated in unit dosage form.

71. The pharmaceutical composition of claim 70, wherein said unit dosage form is a tablet, caplet, capsule, lozenge, film, strip, gelcap, or syrup.

72. A method to treat a condition modulated by calcium channel activity, said method comprising administering to a subject in need of such treatment an effective amount of the compound of claim 1, or a pharmaceutically acceptable salt, solvate, or prodrug thereof, or a stereoisomer thereof, or a conjugate thereof, or the pharmaceutical composition thereof.

73. The method of claim 72, wherein said calcium channel is a T-type calcium channel.

74. The method of claim 73, wherein said calcium channel is the Ca_{V} 3.1, Ca_{V} 3.2, or Ca_{V} 3.3 channel.

75. The method of claim 72, wherein said condition is pain, epilepsy, Parkinson's disease, depression, psychosis, or tinnitus.

76.-77. (canceled)

78. The method of claim 77, wherein said pain is inflammatory pain, neuropathic pain, or chronic pain.

79. (canceled)

80. The method of claim 79, wherein said chronic pain is peripheral neuropathic pain; central neuropathic pain, musculoskeletal pain, headache, visceral pain, or mixed pain.

81. The method of claim 80, wherein
said peripheral neuropathic pain is post-herpetic neuralgia, diabetic neuropathic pain, neuropathic cancer pain, failed back-surgery syndrome, trigeminal neuralgia, or phantom limb pain;
said central neuropathic pain is multiple sclerosis related pain, Parkinson disease related pain, post-stroke pain, post-traumatic spinal cord injury pain, or pain in dementia;
said musculoskeletal pain is osteoarthritic pain and fibromyalgia syndrome; inflammatory pain such as rheumatoid arthritis, or endometriosis;
said headache is migraine, cluster headache, tension headache syndrome, facial pain, or headache caused by other diseases;
said visceral pain is interstitial cystitis, irritable bowel syndrome, or chronic pelvic pain syndrome; or
said mixed pain is lower back pain, neck and shoulder pain, burning mouth syndrome, or complex regional pain syndrome.

82. The method of claim 80, wherein said headache is migraine.

83. The method of claim 77, wherein said pain is acute pain.

84. The method of claim 83, wherein said acute pain is nociceptive pain or post-operative pain.

85. (canceled)
